SEQUENCE LISTING (22 sequences)

SEQ ID NO: 1 - protein, 5 aa, Artificial Sequence
  Heavy Chain CDR1
DSNIH

SEQ ID NO: 2 - protein, 17 aa, Artificial Sequence
  Heavy Chain CDR2
YIYPYNGGTDYNQKFKN

SEQ ID NO: 3 - protein, 7 aa, Artificial Sequence
  Heavy Chain CDR3
GNPWLAY

SEQ ID NO: 4 - protein, 116 aa, Artificial Sequence [sequence omitted]

SEQ ID NO: 5 - protein, 15 aa, Artificial Sequence
  Light Chain CDR1
RASESLDNYGIRFLT

SEQ ID NO: 6 - protein, 8 aa, Artificial Sequence
  Light Chain CDR2
AASNQGSG

SEQ ID NO: 7 - protein, 9 aa, Artificial Sequence
  Light Chain CDR3
QQTKEVPWS

SEQ ID NO: 8 - protein, 111 aa, Artificial Sequence [sequence omitted]

SEQ ID NO: 9 - protein, 446 aa, Artificial Sequence
  CD33AB Heavy Chain
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, Artificial Sequence
  CD33AB Light Chain
DIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 443 aa, Artificial Sequence
  Gemtuzumab Heavy Chain
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 218 aa, Artificial Sequence; identical to SEQ ID NO: 10
  Gemtuzumab Light  Chain

SEQ ID NO: 13 - protein, 5 aa, Artificial Sequence
  huMy9-6 Heavy Chain CDR1
SYYIH

SEQ ID NO: 14 - protein, 17 aa, Artificial Sequence
  huMy9-6 Heavy Chain CDR2
VIYPGNDDISYNQKFQG

SEQ ID NO: 15 - protein, 9 aa, Artificial Sequence
  huMy9-6 Heavy Chain CDR3
EVRLRYFDV

SEQ ID NO: 16 - protein, 118 aa, Artificial Sequence
  huMy9-6 Heavy Chain Variable Region
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 17 - protein, 17 aa, Artificial Sequence
  huMy9-6 Light Chain CDR1
KSSQSVFFSSSQKNYLA

SEQ ID NO: 18 - protein, 7 aa, Artificial Sequence
  huMy9-6 Light Chain CDR2
WASTRES

SEQ ID NO: 19 - protein, 8 aa, Artificial Sequence
  huMy9-6 Light Chain CDR3
HQYLSSRT

SEQ ID NO: 20 - protein, 112 aa, Artificial Sequence
  huMy9-6 Light Chain Variable Region
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 21 - protein, 445 aa, Artificial Sequence
  huMy9-6-IgG4-S228P Heavy Chain
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 219 aa, Artificial Sequence
  huMy9-6-IgG4-S228P Light Chain
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2025/0090681 A1
(43) Pub. Date: Mar. 20, 2025

(54) NEODEGRADER CONJUGATES

(71) Applicants: Orum Therapeutics, Inc., Daejeon (KR); BRISTO-MYERS SQUIBB COMPANY, Lawrenceville, NJ (US)

(72) Inventors: Nathan FISHKIN, Cambridge, MA (US); Peter U. PARK, Cambridge (MA); Chen BAI, Cambridge, MA (US)

(21) Appl. No.: 18/566,822

(22) PCT Filed: Jun. 2, 2022
(86) PCT No.: PCT/IB2022/055167

§ 371 Date: Dec. 4, 2023

Related U.S. Application Data
(60) Provisional application No. 63/202,273, filed on Jun. 3, 2021.
(60) Provisional application No. 63/282,585, filed on Nov. 23, 2021.

(51) Int. Cl. A61K 47/68; A61K 47/54; C07K 16/28

(52) U.S. Cl. CPC A61K 47/6889; A61K 47/549; A61K 47/6803; A61K 47/6849; C07K 16/2803

(57) ABSTRACT

The present disclosure provides neoDegraders and neoDegraders conjugated to binding moieties. Also provided are compositions comprising the conjugates. The compounds and compositions are useful for treating a disease or condition, e.g., cancer, in a subject in need thereof.

### REFERENCE TO SEQUENCE LISTING SUBMITTED ELECTRONICALLY

The content of the electronically submitted sequence listing in ASCII text file (Name 4547_016PC02_Seglisting_ST25; Size: 24,777 bytes; and Date of Creation: May 31, 2022) filed with the application is incorporated herein by reference in its entirety.

### FIELD

The present disclosure provides neoDegrader conjugates, wherein the neoDegrader is conjugated to a binding moiety. Also provided are compositions comprising the conjugates. The conjugates and compositions are useful for treating cancer in a subject in need thereof.

### BACKGROUND

Protein degradation has been validated as a therapeutic strategy by the effectiveness of immunomodulatory imide drugs. These compounds have the ability to bind to cereblon (CRBN) and promote recruitment and ubiquitination of substrate proteins mediated by CRL4^{CRBN} E3 ubiquitin ligase. It is thought that immunomodulatory imides act as “molecular glues,” filling the binding interface as a hydrophobic patch that reprograms protein interactions between the ligase and neosubstrates.

Despite the excitement for these compounds as novel treatments for cancer, thus far they have been limited to use in hematologic malignancies such as multiple myeloma and myelodysplastic syndrome (MDS). Expanding the library of compounds that can function by degrading other oncoproteins, many of which have been considered ‘undruggable,’ is an active area of drug development. Thus there is a continuing need for new compounds that can target these alternative oncoproteins and treat a wide array of cancers.

### SUMMARY

Treatment of patients with cancers with small-molecule GSPT1 degraders has been shown to drive clinical responses, but has been associated with severe adverse events (AE). Cancers frequently express antigens on their surfaces that are not expressed, or ar expressed at much lower leves, on healthy cells. The present invention is based on the discovery that combining a GSPT1 degrading payload molecule with an antibody that binds to cell surface antigen on a cancer cell can improve both the clinical efficacy and tolerability of a GSPT1 degrader.

In certain aspects, the present disclosure provides a conjugate of formula (I):

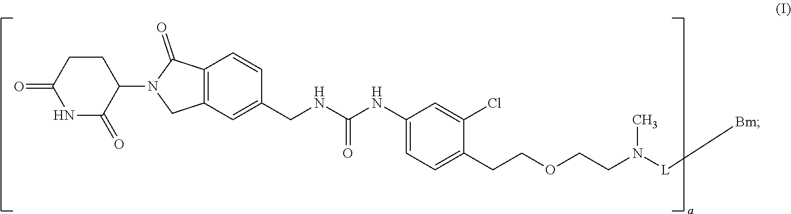

or a pharmaceutically acceptable salt thereof, wherein:
   a is 1 to 10;
   L is a linker selected from

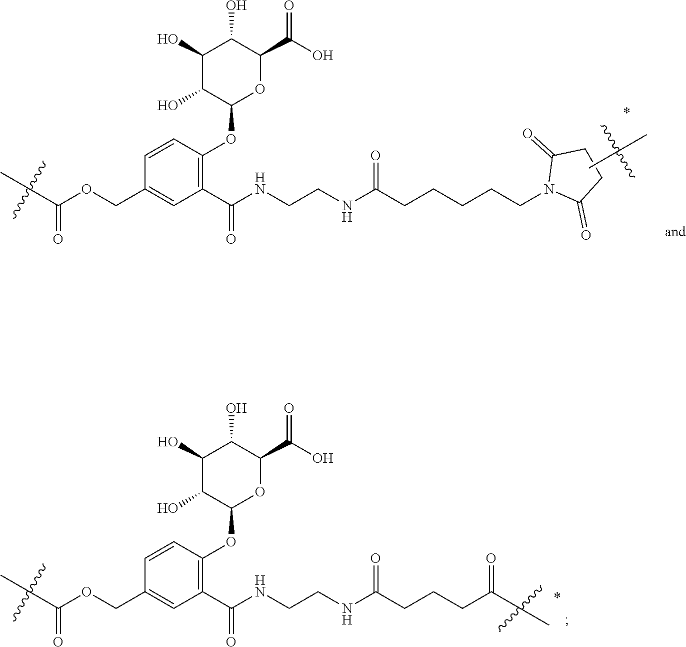

wherein:
   [symbol] is the point of attachment to the nitrogen atom; and
   [symbol] is the point of attachment to Bm; and
   Bm is a binding moiety that is capable of specifically binding to a protein, e.g., a protein that is a cell surface antigen.

In certain aspects, the binding moiety is an antibody, antibody fragment, or an antigen-binding fragment. In some aspects, a is 2 to 8.

In some aspects, L is

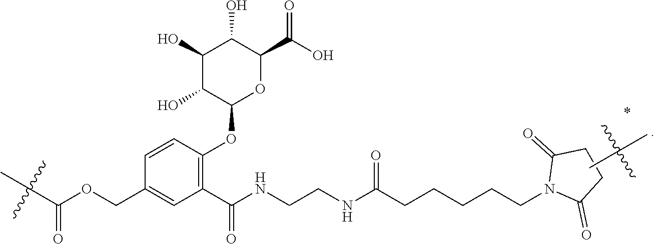

In some aspects, L is

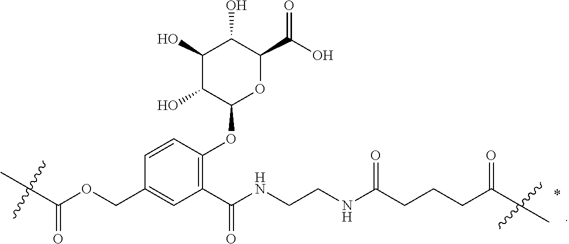 [SMILES: *C(=O)CCCC(=O)NCCNC(=O)c1cc(COC(*)=O)ccc1O[C@@H]1O[C@H](C(=O)O)[C@@H](O)[C@H](O)[C@H]1O]

In some aspects, Bm is an antibody or antigen binding portion thereof. In some aspects, the protein that the binding moiety binds to is a surface antigen. In some aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, c13, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI24, HMWMAA, HPV E6, hTERT, ICAM, ICOS-L, IFN-α, IFN-γ, IGF-I receptor, IGLL1, IL-2 receptor (IL-2Rα (i.e., CD25), IL-2Rβ (i.e., CD122), IL-2Rγ (i.e., CD132)), IL-4 receptor (IL-4R, IL-2Rγ/IL-13Rα1), IL-13 receptor (IL-13Rα1, IL-13Rα2, IL-4R) IL-1 receptor (IL-11Ra), IL-12 receptor (IL-12Rβ1, IL-12Rβ2), IL-23 receptor (IL-12Rβ1, IL-23R), IL-22 receptor (IL-22Rα1, IL-22Rα2, IL-10Rβ), IL-5 receptor (IL-5Rα, CSF2RB), IL-6 receptor (IL-6Rα, gp130), interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostein, survivin and telomerase, PRSS21, PSCA, PSMA, PTK7, RAGE-1, RANKL, Ras, Ras mutant, Rhesus factor, RhoC, RON, ROR1, ROR2, RU1, RU2, SART3, SLAMF7, SLC44A4, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR4, GD2, PDGFR, TEM1/CD248, TROP-2, or combinations thereof. In some aspects, the surface antigen comprises CD33.

In some aspects, the antibody is selected from the group consisting of rituximab, trastuzumab, gemtuzumab, CD33AB, pertuzumab, obinutuzumab, ofatumumab, olaratumab, ontuximab, isatuximab, Sacituzumab, U3-1784, daratumumab, STI-6129, lintuzumab, huMy9-6, huMy9-6-IgG4-S228P, balantamab, indatuximab, cetuximab, dinutuximab, anti-CD38 A2 antibody, HuAT13/5 antibody, alemtuzumab, ibritumomab, tositumomab, bevacizumab, panitumumab, tremelimumab, ticilimumab, catumaxomab, oregovomab, and veltuzumab.In some aspects, the antibody is rituximab, trastuzumab, pertuzumab, huMy9-6, huMy9-6-IgG4-S228P, CD33AB, lintuzumab, or gemtuzumab. In some aspects, the antibody or antigen binding portion thereof comprises a heavy chain variable region (VH) complementarity determining region (CDR) 1 (VH-CDR1), comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a light chain variable region (VL) CDR1 (VL-CDR1) comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7. In some aspects, the antibody or antigen binding portion thereof comprises a VH comprising the amino acid sequence as set forth in SEQ ID NO: 4 and a VL comprising the amino acid sequence as set forth in SEQ ID NO: 8. In some aspects, the antibody comprises a heavy chain comprising the amino acid sequence as set forth in SEQ ID NO :9 and a light chain comprising the amino acid sequence as set forth in SEQ ID NO: 10. In some aspects, the antibody is CD33AB.

In certain aspects, the present disclosure provides a compound of formula (II):

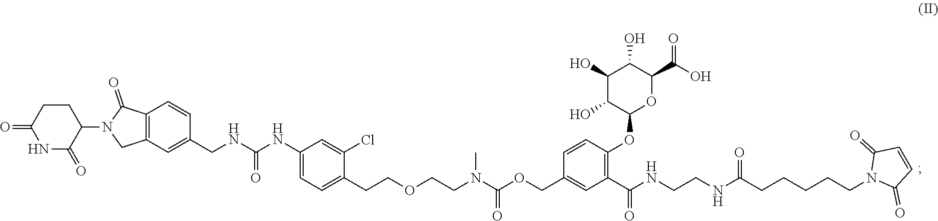 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

or a pharmaceutically acceptable salt thereof.

In certain aspects, the present disclosure provides a compound of formula (III):

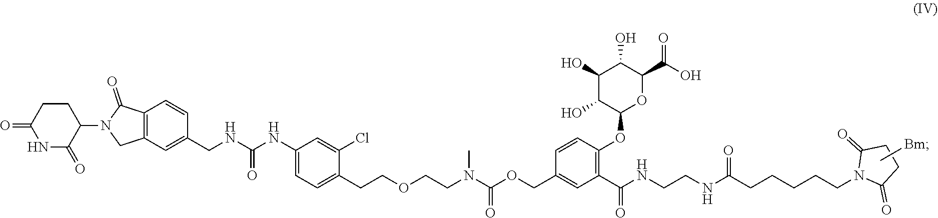

or a pharmaceutically acceptable salt thereof.

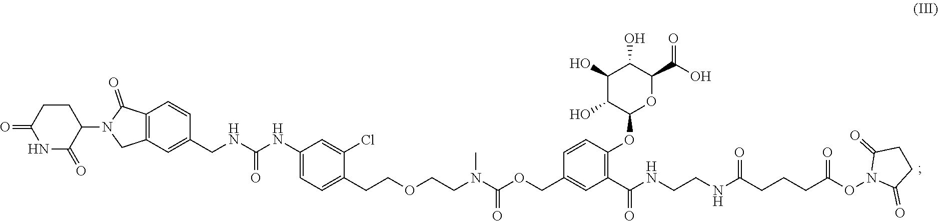 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCC(=O)ON2C(=O)CCC2=O)c1]

In certain aspects, the present disclosure provides a compound of formula (IV):

or a pharmaceutically acceptable salt thereof, wherein Bm is a binding moiety that specifically binds to a protein, e.g, a protein that is a cell surface antigen.

In certain aspects, the present disclosure provides a compound of formula (V):

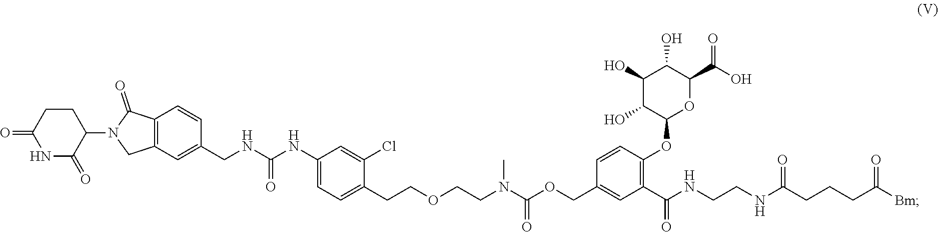

or a pharmaceutically acceptable salt thereof, wherein Bm is a binding moiety that specifically binds to a protein, e.g, a protein that is a cell surface antigen.

In certain aspects, the present disclosure provides compounds of formula (IV) or (V), wherein Bm is an antibody or antigen binding portion thereof. In certain aspects, the protein that the binding moiety specifically binds to is a surface antigen. In some aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, ICAM, ICOS-L, IFN-α, IFN-γ, IGF-I receptor, IGLL1, IL-2 receptor (IL-2Rα (i.e., CD25), IL-2Rβ (i.e., CD122), IL-2Rγ (i.e., CD132)), IL-4 receptor (IL-4R, IL-2Rγ/IL-13Rα1), IL-13 receptor (IL-13Rα1, IL-13Rα2, IL-4R) IL-1 receptor (IL-11Ra), IL-12 receptor (IL-12Rβ1, IL-12Rβ2), IL-23 receptor (IL-12Rβ1, IL-23R), IL-22 receptor (IL-22Rα1, IL-22Rα2, IL-10βP), IL-5 receptor (IL-5Ra, CSF2RB), IL-6 receptor (IL-6Ra, gp130), interferon receptor, integrins (including α₄, αᵥβ₃, αᵥβ₅, αᵥβ₆, α₁β4, α₄β₁, α₄β₇, α₁β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostein, survivin and telomerase, PRSS21, PSCA, PSMA, PTK7, RAGE-1, RANKL, Ras mutant, Rhesus factor, RhoC, RON, ROR1, ROR2, RU1, RU2, SART3, SLAMF7, SLC44A4, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR, GD2, PDGFR, TEM1/CD248, TROP-2, or combinations thereof.

In some aspects, the antibody comprises rituximab, trastuzumab, gemtuzumab, pertuzumab, obinutuzumab, ofatumumab, olaratumab, ontuximab, isatuximab, sacituzumab, U3-1784, daratumumab, STI-6129, lintuzumab, huMy9-6, huMy9-6-IgG4-S228P, balantamab, indatuximab, cetuximab, dinutuximab, anti-CD38 A2 antibody, HuAT13/5 antibody, CD33AB, alemtuzumab, ibritumomab, tositumomab, bevacizumab, panitumumab, tremelimumab, ticilimumab, catumaxomab, oregovomab, or veltuzumab.

In some aspects, the antibody is rituximab, trastuzumab, pertuzumab, huMy9-6, huMy9-6-IgG4-S228P, CD33AB, lintuzumab, or gemtuzumab. In some aspects, the antibody or antigen binding portion thereof comprises a VH-CDR1, comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7. In some aspects, the antibody or antigen binding portion thereof comprises a VH comprising the amino acid sequence as set forth in SEQ ID NO: 4 and a VL comprising the amino acid sequence as set forth in SEQ ID NO: 8. In some aspects, the antibody comprises a heavy chain comprising the amino acid sequence as set forth in SEQ ID NO :9 and a light chain comprising the amino acid sequence as set forth in SEQ ID NO: 10. In some aspects, the antibody is CD33AB.

In some aspects, the present disclosure provides a pharmaceutical composition comprising a conjugate or compound as described above, or a pharmaceutically acceptable salt thereof, and one or more pharmaceutically acceptable carriers.

In some aspects, the present disclosure provides a method of treating cancer or myelodysplastic syndrome (MDS) in a subject in need thereof, the method comprising administering to the subject a pharmaceutically acceptable amount of a conjugate, compound, or composition as described above, or a pharmaceutically acceptable salt thereof. In some aspects, the cancer is a solid tumor. In some aspects, the cancer is a hemalotologic/blood cancer. In some aspects, the cancer is breast cancer, gastric cancer, lymphoma, acute myeloid leukemia, multiple myeloma, head and neck cancer, squamous cell carcinoma, and/or hepatocellular carcinoma. In some aspects, the cancer is refractory or resistant to Mylotarg.

In some aspects, the method further comprises administering to the subject a pharmaceutically acceptable amount of an additional agent prior to, after, or simultaneously with the conjugate or compound, or a pharmaceutically acceptable salt thereof. In some aspects, the additional agent is a cytotoxic agent or an immune response modifier. In some aspects, the immune response modifier is a checkpoint inhibitor. In some aspects, the checkpoint inhibitor comprises a PD-1 inhibitor, a PD-L1 inhibitor, a CTLA-4 inhibitor, a TIM3 inhibitor, and/or a LAG-3 inhibitor.

In certain aspects, the present disclosure provides a method of preparing the conjugate of formula (I), or a pharmaceutically acceptable salt thereof, the process comprising reacting a binding moiety with a compound of formula (I-1):

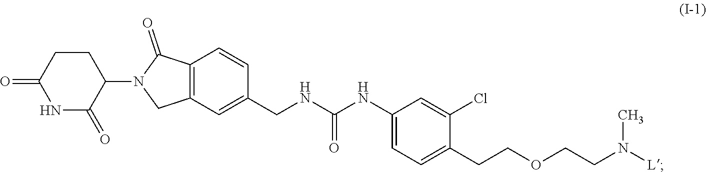

or a pharmaceutically acceptable salt thereof, wherein:
L is selected from

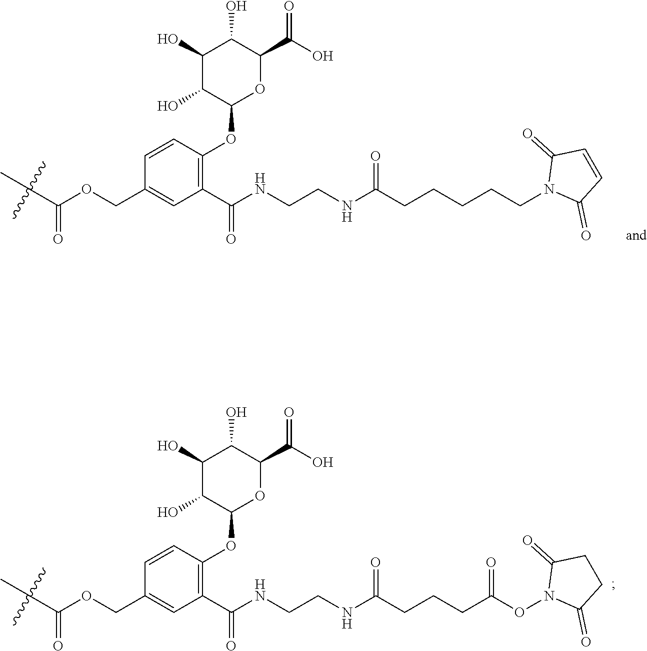 [SMILES: *C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1 ; *C(=O)OCc1ccc(O[C@@H]2O[C@H](C(=O)O)[C@@H](O)[C@H](O)[C@H]2O)c(C(=O)NCCNC(=O)CCCC(=O)ON2C(=O)CCC2=O)c1]

wherein:
[symbol] is the point of attachment to the nitrogen atom.

In some aspects, the method further comprises reducing the binding moiety prior to reacting with the compound of formula (I-1).

In some aspects, L′ is

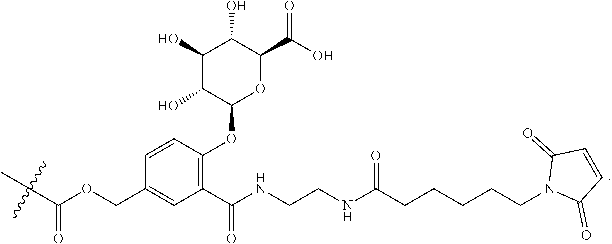 [SMILES: *C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

In some aspects, L′ is

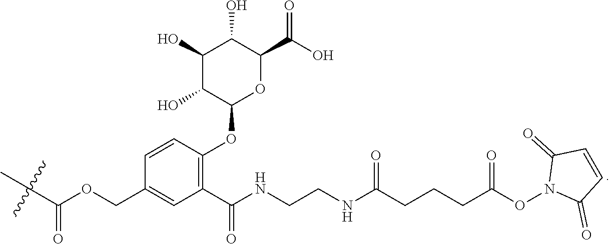 [SMILES: *C(=O)OCc1ccc(O[C@@H]2O[C@H](C(=O)O)[C@@H](O)[C@H](O)[C@H]2O)c(C(=O)NCCNC(=O)CCCC(=O)ON2C(=O)C=CC2=O)c1]

In some aspects, the compound of formula (I-1) is reacted with a binding moiety, which comprises an antibody or an antigen binding portion thereof.

In some aspects, the antibody or antigen binding portion thereof binds to a surface antigen. In some aspects, the surface antigen comprises 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2,ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, ICOS-L, IFN-α, IFN-γ, IGF-I receptor, IGLL1, IL-2 receptor (IL-2Rα (i.e., CD25), IL-2Rβ (i.e., CD122), IL-2RT (i.e., CD132)), IL-4 receptor (IL-4R, IL-2Rγ/IL-13Rα1), IL-13 receptor (IL-13Rα1, IL-13Rα2, IL-4R) IL-1 receptor (IL-1 1Ra), IL-12 receptor (IL-12Rβ1, IL-12Rβ2), IL-23 receptor (IL-12Rβ1, IL-23R), IL-22 receptor (IL-22Rα1, IL-22Rα2, IL-10Rβ), IL-5 receptor (IL-5Rα, CSF2RB), IL-6 receptor (IL-6Rα, gp130), interferon receptor, integrins (including α₄, αᵥβ₃, αᵥβ₅, αᵥβ₆, α₁β₄, α₄β₁, α₄β₇, α₅β₁, α₆β₄, α_{IIb}β₃ intergins), Integrin alphaV, intestinal carboxyl esterase, KIT, LAGE-1a, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostein, survivin and telomerase, PRSS21, PSCA, PSMA, PTK7, RAGE-1, RANKL, Ras mutant, Rhesus factor, RhoC, RON, ROR1, ROR2, RU1, RU2, SART3, SLAMF7, SLC44A4, SLITRK6, sperm protein 17, sphingosine-1-phosphate, SSEA-4, SSX2, STEAP1, TAG72, TARP, TCRβ, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, XAGE1, or combinations thereof.

In some aspects, the surface antigen comprises HER2, CD20, CD38, CD33, BCMA, CD138, EGFR, FGFR4, GD2, PDGFR, TEM1/CD248, Trop-2 or combinations thereof.

In some aspects, the antibody comprises rituximab, trastuzumab, gemtuzumab, pertuzumab, obinutuzumab, ofatumumab, olaratumab, ontuximab, isatuximab, sacituzumab, U3-1784, daratumumab, STI-6129, lintuzumab, huMy9-6, huMy9-6-IgG4-S228P, balantamab, indatuximab, cetuximab, dinutuximab, anti-CD38 A2 antibody, CD33AB, HuAT13/5 antibody, alemtuzumab, ibritumomab, tositumomab, bevacizumab, panitumumab, tremelimumab, ticilimumab, catumaxomab, oregovomab, or veltuzumab.

In some aspects, the antibody is rituximab, trastuzumab, pertuzumab, huMy9-6, huMY9-6-IgG4-S228P, CD33AB, lintuzumab, or gemtuzumab. In some aspects, the antibody or antigen binding portion thereof comprises a VH-CDR1, comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a VL-CDR1 comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7. In some aspects, the antibody or antigen binding portion thereof comprises a VH comprising the amino acid sequence as set forth in SEQ ID NO: 4 and a VL comprising the amino acid sequence as set forth in SEQ ID NO: 8. In some aspects, the antibody comprises a heavy chain comprising the amino acid sequence as set forth in SEQ ID NO: 9 and a light chain comprising the amino acid sequence as set forth in SEQ ID NO: 10.

### BRIEF DESCRIPTION OF THE FIGURES

FIG. 1A depicts in vivo activity of representative neoDegrader conjugates against MV411 (CD33+) tumors. The X axis shows the day after dosing. The Y axis shows the tumor volume (mm³) after dosing with vehicle, 3.02 mg/kg CD33AB—Compound (Ia), 2.94 mg/kg CD33AB—Compound (Ib), 0.1 mg/kg Mylotarg, 50 mg/kg×21 Venetoclax, or 5 mg/kg bid×10 of CC-90009.

FIG. 1B depicts in vivo activity of representative neoDegrader conjugates against MV411 (CD33+) tumors. The X axis shows the day after dosing. The Y axis shows the tumor volume (mm³) after dosing with vehicle, 3 mg/kg CD33AB—Compound (Ic), 2.83 mg/kg CD33AB—Compound (Ie), 3.02 mg/kg CD33AB—Compound (Ia), 2.99 mg/kg CD33AB—Compound (Ih), 2.94 mg/kg CD33AB—Compound (Ib), 0.1 mg/kg Mylotarg, 50 mg/kg×21 Venetoclax, or 5 mg/kg bid×10 of CC-90009.

FIG. 2 depicts the in vitro activity of CD33AB-Compound (Ia), CC-90009, and Mylotarg against AML (CD33+) cells and non-AML (CD33-) cells.

FIG. 3 depicts the in vitro activity of the CD33AB—Compound (Ia) conjugate against MV4-11 (CD33+) cells. The X axis shows concentration. The Y axis shows the % cell viability after dosing with a non-CD33 binding antibody—neoDegrader conjugate, CD33AB, neoDegrader P1, Venetoclax, CC-885, CC-90009, Mylotarg, and CD33AB—Compound (Ia).

FIG. 4 depicts the in vitro activity of the CD33AB—Compound (Ia) conjugate, Mylotarg, and CC-90009 against patient-derived primary relapsed/refractory AML cells.

FIG. 5 depicts the in vitro activity of the CD33AB—Compound (Ia) conjugate, CC-90009, and Mylotarg in normal erythroid, myeloid, and megakaryocyte progenitors as measured in colony forming cell (CFC) assays.

FIG. 6 depicts the in vivo activity of CD33AB—Compound (Ia) against MV4-11 (CD33+) tumors. In the left graph, the X axis shows the day after dosing, and the Y axis shows the tumor volume (mm³) after dosing with vehicle, 5 mg/kg bid CD-90009, 0.1 mg/kg Mylotarg, 50 mg/kg qd Venetoclax, and 3 mg/kg CD33AB—Compound (Ia). In the right graph, the X axis shows the day after dosing, and the Y axis shows the tumor volume (mm³) after dosing with vehicle, 3 mg/kg CD33 non-binding antibody neoDegrader conjugate, 8 mg/kg azacitidine and 50 mg/kg venetoclax, 1 mg/kg CD33AB—Compound (Ia), and 3 mg/kg CD33AB—Compound (Ia).

FIG. 7 depicts the in vivo activity of CD33AB—Compound (Ia) in MV4-11 and OCI-AML2 cells.

FIG. 8 is a Western blot showing the degradation of GSPT 1 after in vitro treatment of MV4-11 cells with the CD33AB—Compound (Ia) conjugate. GSPT1 is completely degraded in 12 hours when treated with CD33AB—Compound (Ia) conjugate.

FIG. 9 depicts the sustained in vitro effect of CD33AB—Compound (Ia) on GSPT1 (top) and the pharmacokinetics of CD33AB—Compound (Ia) conjugate (QD×1 IV) (bottom).

FIGS. 10A and 10B depict the in vitro activity of the CD33AB-Compound (Ia) conjugate against Mylotarg-insensitive AML cells (AML-193 (FIG. 10A) and Kasumi-6 (FIG. 10B). The X axis shows concentration and the Y axis shows the percent viability of the cell line after treatment.

### DETAILED DESCRIPTION

The present disclosure is directed to a conjugate of formula (I):

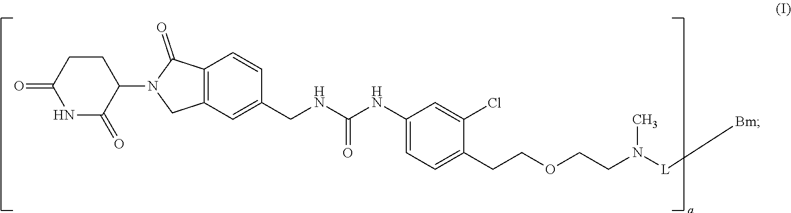

or a pharmaceutically acceptable salt thereof, wherein:
   a is 1 to 10;
   L is a linker selected from

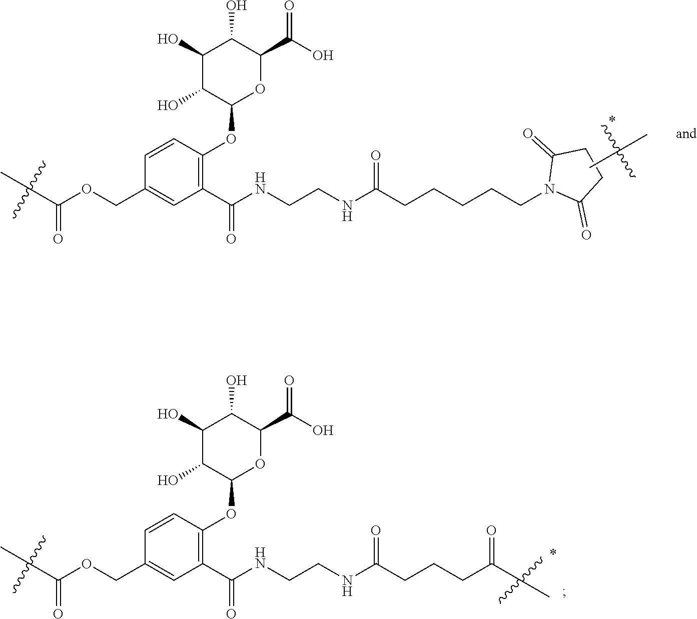

wherein:
   [symbol] is the point of attachment to the nitrogen atom; and
   [symbol] is the point of attachment to Bm; and
   Bm is a binding moiety that is capable of specifically binding to a protein. In some aspects, the binding moiety is an antibody, antibody fragment, or an antigen-binding fragment.

The present disclosure also provides the compound above that is fused to the binding moiety, the composition comprising the compound or the conjugate, or the method of using or making the compound or the conjugate.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Systéme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “DAR,” as used herein, refers to the drug antibody ratio of the conjugate, which is the average number of neoDegrader-linker complexes linked to each antibody. In certain aspects, the DAR of the conjugates described herein is from 1 to 10. In some aspects, the DAR of the conjugates described herein is from 1 to 8. In some aspects, the DAR of the conjugates described herein is 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.

The term “antibody,” as used herein, also refers to a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “single domain antibody,” also known as a nanobody, is an antibody fragment consisting of a single monomeric variable antibody domain with a molecular weight of from about 12 kDa to about 15 kDa. Single body antibodies can be based on heavy chain variable domains or light chains. Examples of single domain antibodies include, but are not limited to, V_{H}H fragments and V_{NAR} fragments.

“Antibody fragments” comprise a portion of an intact antibody, generally the antigen binding or variable region thereof. Examples of antibody fragments include Fab, Fab′, F(ab′).sub.2, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

An “intact antibody” is one which comprises an antigen-binding variable region as well as a light chain constant domain (CL) and heavy chain constant domains, CH1, CH2 and CH3. The constant domains may be native sequence constant domains (e.g., human native sequence constant domains) or amino acid sequence variant thereof.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method, or may be made by recombinant DNA methods. The “monoclonal antibodies” may also be isolated from phage antibody libraries.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies of interest herein include “primatized” antibodies comprising variable domain antigen-binding sequences derived from a non-human primate (e.g., Old World Monkey, Ape etc.) and human constant region sequences.

Various methods have been employed to produce monoclonal antibodies (MAbs). Hybridoma technology, which refers to a cloned cell line that produces a single type of antibody, uses the cells of various species, including mice (murine), hamsters, rats, and humans. Another method to prepare MAbs uses genetic engineering including recombinant DNA techniques. Monoclonal antibodies made from these techniques include, among others, chimeric antibodies and humanized antibodies. A chimeric antibody combines DNA encoding regions from more than one type of species. For example, a chimeric antibody may derive the variable region from a mouse and the constant region from a human. A humanized antibody comes predominantly from a human, even though it contains nonhuman portions. Like a chimeric antibody, a humanized antibody may contain a completely human constant region. But unlike a chimeric antibody, the variable region may be partially derived from a human. The nonhuman, synthetic portions of a humanized antibody often come from CDRs in murine antibodies. In any event, these regions are crucial to allow the antibody to recognize and bind to a specific antigen. While useful for diagnostics and short-term therapies, murine antibodies cannot be administered to people long-term without increasing the risk of a deleterious immunogenic response. This response, called Human Anti-Mouse Antibody (HAMA), occurs when a human immune system recognizes the murine antibody as foreign and attacks it. A HAMA response can cause toxic shock or even death.

Chimeric and humanized antibodies reduce the likelihood of a HAMA response by minimizing the nonhuman portions of administered antibodies. Furthermore, chimeric and humanized antibodies can have the additional benefit of activating secondary human immune responses, such as antibody dependent cellular cytotoxicity.

The intact antibody may have one or more “effector functions” which refer to those biological activities attributable to the Fc region (a native sequence Fc region or amino acid sequence variant Fc region) of an antibody. Examples of antibody effector functions include Clq binding; complement dependent cytotoxicity; Fc receptor binding; antibody-dependent cell-mediated cytotoxicity (ADCC); phagocytosis; down regulation of cell surface receptors (e.g., B cell receptor; BCR), etc.

Depending on the amino acid sequence of the constant domain of their heavy chains, intact antibodies can be assigned to different “classes”. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The terms “administration,” “administering,” and grammatical variants thereof refer to introducing a composition, such as an EV (e.g., exosome) of the present disclosure, into a subject via a pharmaceutically acceptable route. The introduction of a composition, such as an EV (e.g., exosome) of the present disclosure, into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

As used herein, the term “antibody” encompasses an immunoglobulin whether natural or partly or wholly synthetically produced, and fragments thereof. The term also covers any protein having a binding domain that is homologous to an immunoglobulin binding domain. “Antibody” further includes a polypeptide comprising a framework region from an immunoglobulin gene or fragments thereof that specifically binds and recognizes an antigen. Use of the term antibody is meant to include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

The terms “antibody-drug conjugate” and “ADC” are used interchangeably and refer to an antibody linked, e.g., covalently, to a therapeutic agent (sometimes referred to herein as agent, drug, or active pharmaceutical ingredient) or agents. In some aspects of the present disclosure, the biologically active molecule is an antibody-drug conjugate.

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In some aspects, two or more sequences are said to be “completely conserved” or “identical” if they are 100% identical to one another. In some aspects, two or more sequences are said to be “highly conserved” if they are at least about 70% identical, at least about 80% identical, at least about 90% identical, or at least about 95% identical to one another. In some aspects, two or more sequences are said to be “conserved” if they are at least about 30% identical, at least about 40% identical, at least about 50% identical, at least about 60% identical, at least about 70% identical, at least about 80% identical, at least about 90% identical, or at least about 95% identical to one another. Conservation of sequence can apply to the entire length of an polynucleotide or polypeptide or can apply to a portion, region or feature thereof.

As used herein, the terms “linking” and “conjugating” are used interchangeably an each refer to the covalent or non-covalent attachment of two or more moieties comprising a neoDegrader and a binding moiety. In some aspects the linking or conjugating can comprise a linker.

The term “amino acid sequence variant” refers to polypeptides having amino acid sequences that differ to some extent from a native sequence polypeptide. Ordinarily, amino acid sequence variants will possess at least about 70% sequence identity with at least one receptor binding domain of a native antibody or with at least one ligand binding domain of a native receptor, and typically, they will be at least about 80%, more typically, at least about 90% homologous by sequence with such receptor or ligand binding domains. The amino acid sequence variants possess substitutions, deletions, and/or insertions at certain positions within the amino acid sequence of the native amino acid sequence. Amino acids are designated by the conventional names, one-letter and three-letter codes.

“Sequence identity” is defined as the percentage of residues in the amino acid sequence variant that are identical after aligning the sequences and introducing gaps, if necessary, to achieve the maximum percent sequence identity. Methods and computer programs for the alignment are well known in the art. One such computer program is “Align 2,” authored by Genentech, Inc., which was filed with user documentation in the United States Copyright Office, Washington, D.C. 20559, on Dec. 10, 1991.

The terms “Fc receptor” or “FcR” are used to describe a receptor that binds to the Fc region of an antibody. An exemplary FcR is a native sequence human FcR. Moreover, a FcR may be one which binds an IgG antibody (a gamma receptor) and includes receptors of the Fc.gamma.RI, Fc.gamma.RII, and Fc.gamma. RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Activating receptor Fc.gamma.RIIA contains an immunoreceptor tyrosine-based activation motif (ITAM) in its cytoplasmic domain. Inhibiting receptor Fc.gamma.RIIB contains an immunoreceptor tyrosine-based inhibition motif (ITIM) in its cytoplasmic domain. Other FcRs, including those to be identified in the future, are encompassed by the term “FcR” herein. The term also includes the neonatal receptor, FcRn, which is responsible for the transfer of maternal IgGs to the fetus.

“Complement dependent cytotoxicity” or “CDC” refers to the ability of a molecule to lyse a target in the presence of complement. The complement activation pathway is initiated by the binding of the first component of the complement system (Clq) to a molecule (e.g., an antibody) complexed with a cognate antigen. To assess complement activation, a CDC assay may be performed.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies. The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g., residues 24-34 (L1), 50-56 (L2) and 89-97 (L3) in the light chain variable domain and 31-35 (H1), 50-65 (H2) and 95-102 (H3) in the heavy chain variable domain; Kabat et al supra) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain). “Framework Region” or “FR” residues are those variable domain residues other than the hypervariable region residues as herein defined.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments which have hinge cysteines between them. Other chemical couplings of antibody fragments are also known.

The “light chains” of antibodies from any vertebrate species can be assigned to one of two clearly distinct types, called kappa (κ) and lambda (λ), based on the amino acid sequences of their constant domains.

“Single-chain Fv” or “scFv” antibody fragments comprise the VH and VL domains of antibody, wherein these domains are present in a single polypeptide chain. The Fv polypeptide may further comprise a polypeptide linker between the VH and VL domains which enables the scFv to form the desired structure for antigen binding.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites.

“Humanized” forms of non-human (e.g., rodent) antibodies are chimeric antibodies that contain minimal sequence derived from non-human immunoglobulin. Humanization is a method to transfer the murine antigen binding information to a non-immunogenic human antibody acceptor, and has resulted in many therapeutically useful drugs. The method of humanization generally begins by transferring all six murine complementarity determining regions (CDRs) onto a human antibody framework. These CDR-grafted antibodies generally do not retain their original affinity for antigen binding, and in fact, affinity is often severely impaired. Besides the CDRs, select non-human antibody framework residues must also be incorporated to maintain proper CDR conformation. The transfer of key mouse framework residues to the human acceptor in order to support the structural conformation of the grafted CDRs has been shown to restore antigen binding and affinity. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In certain aspects, the antibody will be purified (1) to greater than 95% by weight of antibody as determined by the Lowry method, or more than 99% by weight, (2) to a degree sufficient to obtain at least 15 residues of N-terminal or internal amino acid sequence by use of a gas phase protein sequencer, or (3) to homogeneity by SDS-PAGE under reducing or nonreducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers.

As used herein, the term “immune response” refers to a biological response within a vertebrate against foreign agents, which response protects the organism against these agents and diseases caused by them. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell. As used herein, the term “T cell” and “T lymphocytes” are interchangeable and refer to any lymphocytes produced or processed by the thymus gland. In some aspects, a T cell is a CD4+ T cell. In some aspects, a T cell is a CD8+ T cell. In some aspects, a T cell is a NKT cell.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent (e.g., neoDegrader or neoDegrader conjugate disclosed herein) that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the composition can, for example, (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer.

In some aspects, a “therapeutically effective amount” is the amount of the neoDegrader or neoDegrader conjugate clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.”

By way of example, an “anti-cancer agent” promotes cancer regression in a subject or prevents further tumor growth. In certain aspects, a therapeutically effective amount of the drug promotes cancer regression to the point of eliminating the cancer.

The terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies.

The terms “treat” or “treatment” refer to both therapeutic treatment and prophylactic or preventative measures, wherein the object is to prevent or slow down (lessen) an undesired physiological change or disorder, such as the development or spread of cancer. For purposes of this disclosure, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### II. NeoDegraders

The present disclosure provides a neoDegrader of formula (P1):

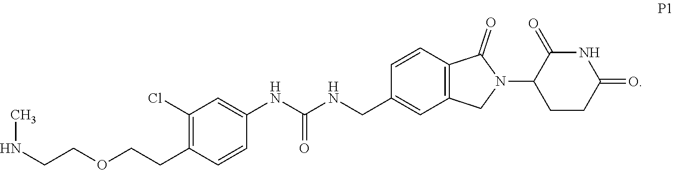 [SMILES: CNCCOCCc1ccc(NC(=O)NCc2ccc3c(c2)CN(C2CCC(=O)NC2=O)C3=O)cc1Cl]

In some aspects, a neoDegrader is a molecule that forms a ternary complex with an E3 ubiquitin ligase which is capable of targeting a protein for degradation.

### III. NeoDegrader Conjugates

The present disclosure provides conjugates of one or more neoDegraders disclosed herein and a binding moiety. These conjugates can degrade proteins by binding to cereblon (CRBN), promoting recruitment and ubiquitination of substrate proteins mediated by CRL4^{CRBN}E3 ubiquitin ligase. These agents act as “molecular glues,” filling the binding interface as a hydrophobic patch that reprograms protein interactions between the ligase and neosubstrates.

In some aspects, the present disclosure provides a compound of formula I

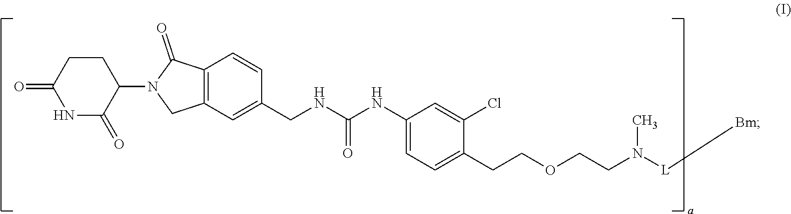

or a pharmaceutically acceptable salt thereof, wherein:
   a is 1 to 10;
   L is a linker selected from

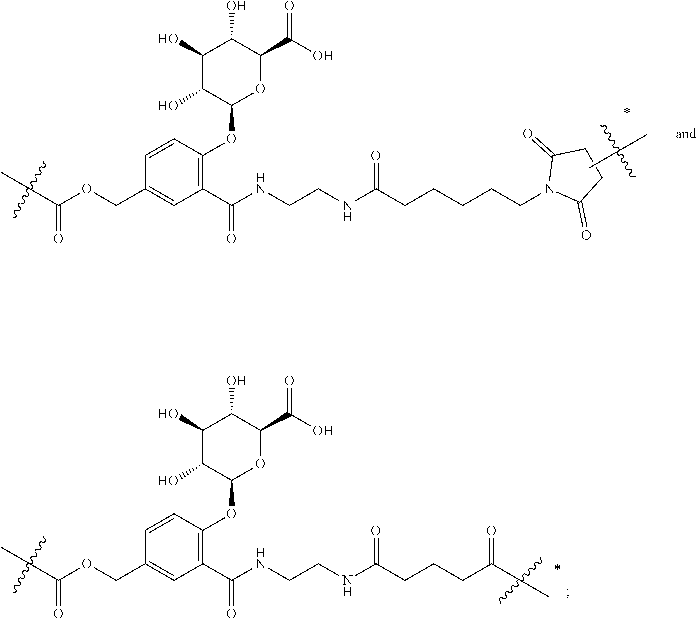

wherein
[symbol] is the point of attachment to the nitrogen atom; and
[symbol] is the point of attachment to Bm; and

In some aspects, the neoDegrader conjugate described herein has in vitro anti-proliferative activity against a tumor cell line. In some aspects, the neoDegrader conjugate comprising a neoDegrader and a binding moiety has in vitro anti-proliferative activity at least about 50%, at least about 60%, at least about 70%, at least about 80%, at least about 90%, at least about 95%, or at least about 100% higher than the neoDegrader alone or the binding moiety alone. In some aspects, the neoDegrader conjugate comprising a neoDegrader and a binding moiety has in vitro anti-proliferative activity at least about 2 fold, at least about 3 fold, at least about 4 fold, at least about 5 fold, at least about 6 fold, at least about 7 fold, at least about 8 fold, at least about 9 fold, at least about 10 fold higher than the neoDegrader alone or the binding moiety alone.

In some aspects, the neoDegrader conjugates described herein have in vitro anti-proliferative activity against a BT-474 breast cancer cell line, e.g., higher anti-proliferative activity against a BT-474 breast cancer cell line, compared to the neoDegrader alone or the binding moiety alone. In some aspects, the neoDegrader conjugates described herein have in vitro anti-proliferative activity against an SK-BR-3 breast cancer cell line, e.g., higher anti-proliferative activity against an SK-BR-3 breast cancer cell line, compared to the neoDegrader alone or the binding moiety alone. In some aspects, the neoDegrader conjugates described herein have in vitro anti-proliferative activity against an NCI-N87 gastric cancer cell line, e.g., higher anti-proliferative activity against a NCI-N87 gastric cancer cell line, compared to the neoDegrader alone or the binding moiety alone. In some aspects, the neoDegrader conjugates described herein have in vitro anti-proliferative activity against a Daudi lymphoma cell line, e.g., higher anti-proliferative activity against a Daudi lymphoma cell line, compared to the neoDegrader alone or the binding moiety alone. In some aspects the neoDegrader conjugates described herein have in vitro anti-proliferative activity against the HL-60 acute myeloid leukemia cell line, e.g., higher anti-proliferative activity against a HL-60 acute myeloid leukemia cell line, compared to the neoDegrader alone or the binding moiety alone. In some aspects, the neoDegrader conjugates described herein have in vitro anti-proliferative activity against a Ramos non-Hodgkins lymphoma cell line, e.g., higher anti-proliferative activity against a Ramos non-Hodgkins lymphoma cell line, compared to the neoDegrader alone or the binding moiety alone. In some aspects, the neoDegrader conjugates described herein have in vitro anti-proliferative activity against an MV411 AML cell line, e.g., higher anti-proliferative activity against an MV411 AML cell line, compared to the neoDegrader alone or the binding moiety alone. In some aspects the neoDegrader conjugates described herein is capable of maintaining their anti-proliferative activity in the presence of human serum. The neoDegrader conjugates described herein can be used in the treatment of cancers.

In some aspects, an antibody neoDegrader conjugate (AnDC) is a conjugate of one or more neoDegraders disclosed herein and an antibody or antigen-binding portion thereof disclosed herein.

### III.A. Linker

The neoDegrader of the present disclosure is linked to the binding moiety via a linker. As used herein, the term “linker” refers to any chemical moiety capable of connecting the binding moiety (Bm) to neoDegrader P1.

In certain aspects, the linker can contain a heterobifunctional group. In the present disclosure, the term “heterobifunctional group” refers to a chemical moiety that connects the linker of which it is a part to the binding moiety. Heterobifunctional groups are characterized as having different reactive groups at either end of the chemical moiety. Attachment to “Bm,” can be accomplished through chemical or enzymatic conjugation, or a combination of both. Chemical conjugation involves the controlled reaction of accessible amino acid residues on the surface of the binding moiety with a reaction handle on the heterobifunctional group. Examples of chemical conjugation include, but are not limited to, lysine amide coupling, cysteine coupling, and coupling via a non-natural amino acid incorporated by genetic engineering, wherein non-natural amino acid residues with a desired reaction handle are installed onto “Bm.” In enzymatic conjugation, an enzyme mediates the coupling of the linker with an accessible amino residue on the binding moiety. Examples of enzymatic conjugation include, but are not limited to, transpeptidation using sortase, transpeptidation using microbial transglutaminase, and N-glycan engineering. Chemical conjugation and enzymatic conjugation may also be used sequentially. For example, enzymatic conjugation can also be used for installing unique reaction handles on “Bm” to be utilized in subsequent chemical conjugation.

In some aspects, the heterobifunctional group is

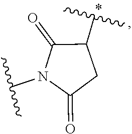 [SMILES: *C1CC(=O)N(*)C1=O]

wherein
[symbol] is the point of attachment to the remaining portion of the linker; and
[symbol] the point of attachment to Bm.

In certain aspects, L is a beta-glucuronidase cleavable linker. In some aspects, L is a beta-glucuronidase cleavable linker selected from:

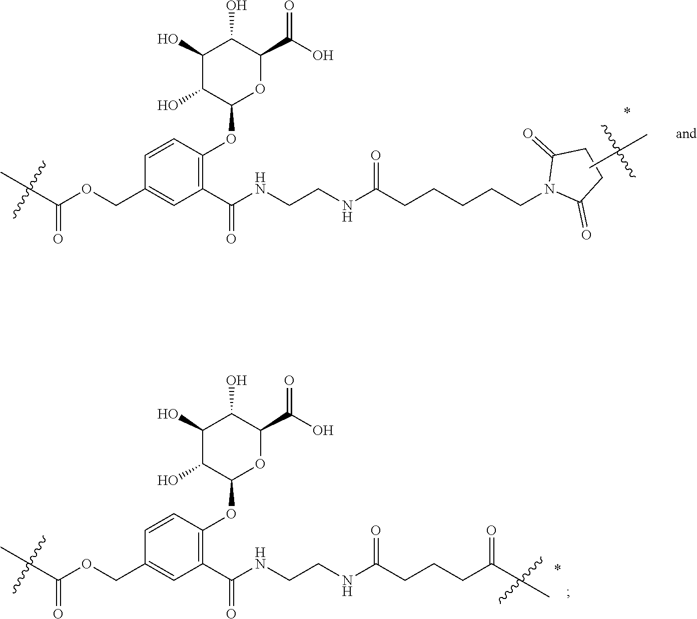

wherein.
[symbol] is the point of attachment to neoDegrader P1; and
[symbol] is the point of attachment to the binding moiety.

### III.B. Binding Moiety

The present disclosure provides neoDegraders conjugated to binding moieties. The term “binding moiety,” as used herein, refers to any molecule that recognizes and binds to a cell surface marker or receptor. In certain aspects, the binding moiety binds to a protein, not limited to a polypeptide moiety. The binding moiety, in addition to targeting the neoDegrader to a specific cell, tissue, or location, may also have certain therapeutic effect such as antiproliferative (cytostatic and/or cytotoxic) activity against a target cell or pathway. In certain aspects the binding moiety can comprise or can be engineered to comprise at least one chemically reactive group such as a carboxylic acid, amine, thiol, or chemically reactive amino acid moiety or side chain. In some aspects, the binding moiety can comprise a targeting moiety which binds or complexes with a cell surface molecule, such as a cell surface receptor or antigen, for a given target cell population. Following specific binding or complexing with the receptor, the cell is permissive for uptake of the targeting moiety or the neoDegrader conjugate, which is then internalized into the cell.

In some aspects, group “Bm” can be a moiety that can specifically bind to a cell surface molecule. In some aspects, group “Bm” can be a peptide or a protein that binds to a cell surface receptor or antigen.

In certain aspects, group “Bm” can be an antibody, antibody fragment, or an antigen-binding fragment. An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. The term “antibody” herein is used in the broadest sense and specifically covers monoclonal antibodies, single domain antibodies, polyclonal antibodies, multispecific antibodies (e.g., bispecific antibodies), and antibody fragments, so long as they exhibit the desired biological activity. Antibodies may be murine, human, humanized, chimeric, or derived from other species.

Monoclonal antibodies that can be conjugated to the neoDegrader are homogeneous populations of antibodies to a particular antigenic determinant (e.g., a cancer cell antigen, a viral antigen, a microbial antigen, a protein, a peptide, a carbohydrate, a chemical, nucleic acid, or fragments thereof). A monoclonal antibody (mAb) to an antigen-of-interest can be prepared by using any technique known in the art which provides for the production of antibody molecules by continuous cell lines in culture. These include, but are not limited to, the hybridoma technique, the human B cell hybridoma technique, and the EBV-hybridoma technique. Such antibodies may be of any immunoglobulin class including IgG, IgM, IgE, IgA, and IgD and any subclass thereof. The hybridoma producing the mAbs of use in this disclosure may be cultivated in vitro or in vivo.

Useful monoclonal antibodies include, but are not limited to, human monoclonal antibodies, humanized monoclonal antibodies, antibody fragments, or chimeric human-mouse (or other species) monoclonal antibodies. Human monoclonal antibodies may be made by any of numerous techniques known in the art.

The antibody can also be a bispecific antibody. Methods for making bispecific antibodies are known in the art. Traditional production of full-length bispecific antibodies is based on the coexpression of two immunoglobulin heavy chain-light chain pairs, where the two chains have different specificities. Because of the random assortment of immunoglobulin heavy and light chains, these hybridomas (quadromas) produce a potential mixture of 10 different antibody molecules, of which only one has the correct bispecific structure. Purification of the correct molecule, which is usually performed using affinity chromatography steps, is rather cumbersome, and the product yields are low.

According to a different approach, antibody variable domains with the desired binding specificities (antibody-antigen combining sites) are fused to immunoglobulin constant domain sequences. The fusion may be with an immunoglobulin heavy chain constant domain, comprising at least part of the hinge, C.sub.H2, and C.sub.H3 regions. The first heavy-chain constant region (C.sub.H1) may contain the site necessary for light chain binding, present in at least one of the fusions. Nucleic acids with sequences encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in aspects when unequal ratios of the three polypeptide chains used in the construction provide the optimum yields. It is, however, possible to insert the coding sequences for two or all three polypeptide chains in one expression vector when the expression of at least two polypeptide chains in equal ratios results in high yields or when the ratios are of no particular significance.

Bispecific antibodies may have a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. Using such techniques, bispecific antibodies can be prepared for conjugation to the neoDegraders in the treatment or prevention of disease as defined herein.

Hybrid or bifunctional antibodies can be derived either biologically, i.e., by cell fusion techniques, or chemically, especially with cross-linking agents or disulfide-bridge forming reagents, and may comprise whole antibodies or fragments thereof.

The antibody can be a functionally active fragment, derivative or analog of an antibody that immunospecifically binds to cancer cell antigens, viral antigens, or microbial antigens or other antibodies bound to tumor cells or matrix. In this regard, “functionally active” means that the fragment, derivative or analog is able to elicit anti-anti-idiotype antibodies that recognize the same antigen that the antibody from which the fragment, derivative or analog is derived recognized. Specifically, in an exemplary aspect the antigenicity of the idiotype of the immunoglobulin molecule can be enhanced by deletion of framework and CDR sequences that are C-terminal to the CDR sequence that specifically recognizes the antigen. To determine which CDR sequences bind the antigen, synthetic peptides containing the CDR sequences can be used in binding assays with the antigen by any binding assay method known in the art.

Other useful antibodies include fragments of antibodies such as, but not limited to, F(ab′)2 fragments, which contain the variable region, the light chain constant region and the CH1 domain of the heavy chain can be produced by pepsin digestion of the antibody molecule, and Fab fragments, which can be generated by reducing the disulfide bridges of the F(ab′)2 fragments. Other useful antibodies are heavy chain and light chain dimers of antibodies, or any minimal fragment thereof such as Fvs or single chain antibodies (SCAs), or any other molecule with the same specificity as the antibody.

Additionally, recombinant antibodies, such as chimeric and humanized monoclonal antibodies, comprising both human and non-human portions, which can be made using standard recombinant DNA techniques, are useful antibodies. A chimeric antibody is a molecule in which different portions are derived from different animal species, such as those having a variable region derived from a murine monoclonal and human immunoglobulin constant regions. Humanized antibodies are antibody molecules from non-human species having one or more complementarity determining regions (CDRs) from the non-human species and a framework region from a human immunoglobulin molecule. Such chimeric and humanized monoclonal antibodies can be produced by recombinant DNA techniques known in the art.

Completely human antibodies can be produced using transgenic mice that are incapable of expressing endogenous immunoglobulin heavy and light chains genes, but which can express human heavy and light chain genes. The transgenic mice are immunized in the normal fashion with a selected antigen, e.g., all or a portion of a polypeptide of the disclosure. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). Other human antibodies can be obtained commercially from, for example, Abgenix, Inc. (Freemont, Calif) and Genpharm (San Jose, Calif).

Completely human antibodies that recognize a selected epitope can be generated using a technique referred to as “guided selection.” In this approach a selected non-human monoclonal antibody, e.g., a mouse antibody, is used to guide the selection of a completely human antibody recognizing the same epitope. Human antibodies can also be produced using various techniques known in the art, including phage display libraries.

The antibody can be a fusion protein of an antibody, or a functionally active fragment thereof, for example in which the antibody is fused via a covalent bond (e.g., a peptide bond), at either the N-terminus or the C-terminus to an amino acid sequence of another protein (or portion thereof, such as at least 10, 20 or 50 amino acid portion of the protein) that is not the antibody. The antibody or fragment thereof may be covalently linked to the other protein at the N-terminus of the constant domain.

Antibodies include analogs and derivatives that are either modified, i.e., by the covalent attachment of any type of molecule as long as such covalent attachment permits the antibody to retain its antigen binding immunospecificity. For example, but not by way of limitation, the derivatives and analogs of the antibodies include those that have been further modified, e.g., by glycosylation, acetylation, pegylation, phosphorylation, amidation, derivatization by known protecting/blocking groups, proteolytic cleavage, linkage to a cellular antibody unit or other protein, etc. Any of numerous chemical modifications can be carried out by known techniques, including, but not limited to specific chemical cleavage, acetylation, formylation, metabolic synthesis in the presence of tunicamycin, etc. Additionally, the analog or derivative can contain one or more unnatural amino acids.

The antibodies in neoDegrader conjugates can include antibodies having modifications (e.g., substitutions, deletions or additions) in amino acid residues that interact with Fc receptors. In particular, antibodies include antibodies having modifications in amino acid residues identified as involved in the interaction between the anti-Fc domain and the FcRn receptor. Antibodies immunospecific for a cancer cell antigen can be obtained commercially, for example, from Genentech (San Francisco, Calif.) or produced by any method known to one of skill in the art such as, e.g., chemical synthesis or recombinant expression techniques. The nucleotide sequence encoding antibodies immunospecific for a cancer cell antigen can be obtained, e.g., from the GenBank database or a database like it, the literature publications, or by routine cloning and sequencing.

In certain aspects, the antibody of the neoDegrader conjugates can be a monoclonal antibody, e.g. a murine monoclonal antibody, a chimeric antibody, or a humanized antibody. In some aspects, the antibody can be an antibody fragment, e.g. a Fab fragment.

Known antibodies for the treatment or prevention of cancer can be conjugated to the neoDegraders described herein. Antibodies immunospecific for a cancer cell antigen can be obtained commercially or produced by any method known to one of skill in the art such as, e.g., recombinant expression techniques. The nucleotide sequence encoding antibodies immunospecific for a cancer cell antigen can be obtained, e.g., from the GenBank database or a database like it, the literature publications, or by routine cloning and sequencing. Examples of antibodies available for the treatment of cancer include, but are not limited to, humanized anti-HER2 monoclonal antibody for the treatment of patients with metastatic breast cancer HERCEPTIN® (trastuzumab); RITUXAN® (rituximab; Genentech) which is a chimeric anti-CD20 monoclonal antibody for the treatment of patients with non-Hodgkin's lymphoma; OvaRex (oregovomab; AltaRex Corporation, MA) which is a murine antibody for the treatment of ovarian cancer; Panorex (edrecolomab, Glaxo Wellcome, NC) which is a murine IgG2a antibody for the treatment of colorectal cancer; Cetuximab Erbitux (cetuximab, Imclone Systems Inc., NY) which is an anti-EGFR IgG chimeric antibody for the treatment of epidermal growth factor positive cancers, such as head and neck cancer; Vitaxin (etaracizumab, MedImmune, Inc., MD) which is a humanized antibody for the treatment of sarcoma; Campath I/H (alemtuzumab, Leukosite, MA) which is a humanized IgG1 antibody for the treatment of chronic lymphocytic leukemia (CLL); Smart MI95 (Protein Design Labs, Inc., CA) which is a humanized anti-CD33 IgG antibody for the treatment of acute myeloid leukemia (AML); LymphoCide (epratuzumab, Immunomedics, Inc., NJ) which is a humanized anti-CD22 IgG antibody for the treatment of non-Hodgkin's lymphoma; Smart ID10 (Protein Design Labs, Inc., CA) which is a humanized anti-HLA-DR antibody for the treatment of non-Hodgkin's lymphoma; Oncolym (Techniclone, Inc., CA) which is a radiolabeled murine anti-HLA-Dr10 antibody for the treatment of non-Hodgkin's lymphoma; Allomune (BioTransplant, CA) which is a humanized anti-CD2 mAb for the treatment of Hodgkin's Disease or non-Hodgkin's lymphoma; Avastin (bevacizumab, Genentech, Inc., CA) which is an anti-VEGF humanized antibody for the treatment of lung and colorectal cancers; Epratuzamab (Immunomedics, Inc., NJ and Amgen, CA) which is an anti-CD22 antibody for the treatment of non-Hodgkin's lymphoma; and CEAcide (Immunomedics, NJ) which is a humanized anti-CEA antibody for the treatment of colorectal cancer.

Other antibodies useful for the neoDegrader conjugates include, but are not limited to, trastuzumab, gemtuzumab, pertuzumab, obinutuzumab, ofatumumab, daratumumab, STI-6129, lintuzumab, huMy9-6, balantamab, indatuximab, dinutuximab, anti-CD38 A2 antibody, HuAT13/5 H3s antibody, ibritumomab, tositumomab, panitumumab, tremelimumab, ticilimumab, catumaxomab, and veltuzumab. In certain aspects, the antibody is selected from the group consisting of rituximab, trastuzumab, pertuzumab, huMy9-6-IgG4-S228P, lintuzumab, and gemtuzumab.

Other antibodies useful for the neoDegrader conjugates include, but are not limited to, antibodies against the following antigens: CA125 (ovarian), CA15-3 (carcinomas), CA19-9 (carcinomas), L6 (carcinomas), Lewis Y (carcinomas), Lewis X (carcinomas), alpha fetoprotein (carcinomas), CA 242 (colorectal), placental alkaline phosphatase (carcinomas), prostate specific antigen (prostate), prostatic acid phosphatase (prostate), epidermal growth factor (carcinomas), MAGE-1 (carcinomas), MAGE-2 (carcinomas), MAGE-3 (carcinomas), MAGE-4 (carcinomas), anti-transferrin receptor (carcinomas), p97 (melanoma), MUC1-KLH (breast cancer), CEA (colorectal), gp100 (melanoma), MART1 (melanoma), PSA (prostate), IL-2 receptor (T-cell leukemia and lymphomas), CD20 (non-Hodgkin's lymphoma), CD52 (leukemia), CD33 (leukemia), CD22 (lymphoma), human chorionic gonadotropin (carcinoma), CD38 (multiple myeloma), CD40 (lymphoma), mucin (carcinomas), P21 (carcinomas), MPG (melanoma), and Neu oncogene product (carcinomas). Some specific, useful antibodies include, but are not limited to, BR96 mAb (Trail, P. A., et al Science (1993) 261, 212-215), BR64 (Trail, P A, et al Cancer Research (1997) 57, 100-105), mAbs against the CD40 antigen, such as S2C6 mAb (Francisco, J. A., et al Cancer Res. (2000) 60:3225-3231), mAbs against the CD70 antigen, such as 1F6 mAb, and mAbs against the CD30 antigen, such as AC10. Many other internalizing antibodies that bind to tumor associated antigens can be used and have been reviewed.

Other antigens that the present conjugates can bind to include, but are not limited to, 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA 125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, clumping factor, cKit, Claudin 3, Claudin 18.2, CLDN6, CLEC12A, CLL-1, cll3, c-MET, Cripto protein, CS1, CTLA-4, CXCR2, CXORF61, Cyclin B1, CYP1B1, Cadherin-3, Cadherin-6, DLL3, E7, EDNRB, EFNA4, EGFR, EGFRvIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GloboH, GM3, GPC1, GPC2, GPC3, gplOO, GPNMB, GPR20, GPRC5D, GUCY2C, HAVCR1, HER2, HER3, HGF, HMI.24, HMWMAA, HPV E6, hTERT, human telomerase reverse transcriptase, ICAM, ICOS-L, IFN-α, IFN-γ, IGF-I receptor, IGLL1, IL-2 receptor (IL-2Rα (i.e., CD25), IL-2Rβ (i.e., CD122), IL-2RT (i.e., CD132)), IL-4 receptor (IL-4R, IL-2Rγ/IL-13Rα1), IL-13 receptor (IL-13Rα1, IL-13Rα2, IL-4R) IL-1 receptor (IL-1 1Ra), IL-12 receptor (IL-12Rβ1, IL-12Rβ2), IL-23 receptor (IL-12Rβ1, IL-23R), IL-22 receptor (IL-22Rα1, IL-22Rα2, IL-10Rβ), IL-5 receptor (IL-5Ra, CSF2RB), IL-6 receptor (IL-6Ra, gp130), interferon receptor, integrins (including α4, αvβ3, αvβ5, αvβ6, α1β4, α4β1, α4β7, α5β1, α6β4, αIIbβ3 intergins), Integrin alphaV, intestinal carboxyl esterase, KIT, LAGE-1a, LAIR1, LAMP-1, LCK, Legumain, LewisY, LFA-1(CD11a), L-selectin(CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, mut hsp70-2, MYCN, myostatin, NA17, NaPi2b, NCA-90, NCAM, Nectin-4, NGF, NOTCH1, NOTCH2, NOTCH3, NOTCH4, NY-BR-1, NY-ESO-1, o-acetyl-GD2, OR51E2, OY-TES1, p53, p53 mutant, PANX3, PAP, PAX3, PAX5, p-CAD, PCTA-1/Galectin 8, PD-L1, PD-L2, PDGFR, PDGFR-beta, phosphatidylserine, PIK3CA, PLAC1, Polysialic acid, Prostase, prostatic carcinoma cell, prostein, Pseudomonas aeruginosa, rabies, survivin and telomerase, PRSS21, PSCA, PSMA, PTK7, RAGE-1, RANKL, Ras mutant, respiratory syncytial virus, Rhesus factor, RhoC, RON, ROR1, ROR2, RU1, RU2, sarcoma translocation breakpoints, SART3, SLAMF7, SLC44A4, sLe, SLITRK6, sperm protein 17, sphingosine-1-phosphate, SSEA-4, SSX2, STEAP1, TAG72, TARP, TCRP, TEM1/CD248, TEM7R, tenascin C, TF, TGF-1, TGF-β2, TNF-α, TGS5, Tie 2, TIM-1, Tn Ag, TRAC, TRAIL-R1, TRAIL-R2, TROP-2, TRP-2, TRPV1, TSHR, tumor antigen CTAA16.88, tyrosinase, UPK2, VEGF, VEGFR1, VEGFR2, vimentin, WT1, and/or XAGE1.

Antibodies that bind to antigens associated with antigen presenting cells such as CD40, OX40L, Endoglin, DEC-205, 4-1BBL, CD36, CD36, CD204, MARCO, DC-SIGN, CLEC9A, CLEC5A, Dectin 2, CLEC10A, CD206, CD64, CD32A, CD1A, HVEM, CD32B, PD-L1, BDCA-2, XCR-1, and CCR2 can also be conjugated to the neoDegraders.

Antibodies of a neoDegrader conjugate can bind to both a receptor or a receptor complex expressed on an activated lymphocyte. The receptor or receptor complex can comprise an immunoglobulin gene superfamily member, a TNF receptor superfamily member, an integrin, a cytokine receptor, a chemokine receptor, a major histocompatibility protein, a lectin, or a complement control protein. Non-limiting examples of suitable immunoglobulin superfamily members are CD2, CD3, CD4, CD8, CD 19, CD22, CD28, CD79, CD90, CD 152/CTLA-4, PD-1, and ICOS. Non-limiting examples of suitable TNF receptor superfamily members are CD27, CD40, CD95/Fas, CD134/OX40, CD137/4-1BB, TNF-R1, TNFR-2, RANK, TACI, BCMA, osteoprotegerin, Apo2/TRAIL-R1, TRAIL-R2, TRAIL-R3, TRAIL-R4, and APO-3. Non-limiting examples of suitable integrins are CD 11a, CD11b, CD 11c, CD18, CD29, CD41, CD49a, CD49b, CD49c, CD49d, CD49e, CD49f, CD 103, and CD 104. Non-limiting examples of suitable lectins are C-type, S-type, and I-type lectin.

In some aspects, the antibodies that can are useful for the present disclosure include, but are not limited to, 3F8, 8H9, abagovomab, abciximab (REOPRO®), abituzumab, abrezekimab, abrilumab, actoxumab, adalimumab (HUMIRA®), adecatumumab, aducanumab, afasevikumab, afelimomab, afutuzumab, alacizumab, ALD518, alemtuzumab (CAMPATH®), alirocumab (PRALUENT®), altumomab, amatuximab, anatumomab, andecaliximab, anetumab, anifrolumab, anrukinzumab, apolizumab, aprutumab, arcitumomab (CEA-SCAN®), ascrinvacumab, aselizumab, atidortoxumab, atlizumab (tocilizumab, ACTEMRA®, ROACTEMRA®), atezolizumab (TECENTRIQ®), atinumab, atorolimumab, avelumab (Bavencio), azintuxizumab, balantamab, bapineuzumab, basiliximab (SIMULECT®), bavituximab, BCD-100, bectumomab (LYMPHOSCAN®), begelomab, belantamab, belimumab (BENLYSTA®), bemarituzumab, benralizumab (FASENRA®), bermekimab, bersanlimab, bertilimumab, besilesomab (SCINITIMUN®), bevacizumab (AVASTIN®), bezlotoxumab (ZINPLAVA®), biciromab (FIBRISCINT®), bimagrumab, bimekizumab, birtamimab, bivatuzumab, bleselumab, blinatumomab, blontuvetmab, blosozumab, bococizumab, brazikumab, brentuximab, briakinumab, brodalumab (SILIQ™), brolucizumab (BEOVU®), brontictuzumab, burosumab (CRYSVITA®), cabiralizumab, caplacizumab (CABLIVIP), camidanlumab, camrelizumab, canakinumab (ILARIS®), cantuzumab, capromab, carlumab, carotuximab, catumaxomab (REMOVAB®), cBR96, CC49, cedelizumab, cemiplimab (LIBTAYO®), cergutuzumab, certrelimab, certolizumab, cetuximab (ERBITUX®), cibisatamab, cirmtuzumab, citatuzumab, cixutumumab, clazakizumab, clenoliximab, clivatuzumab, codrituzumab, cofetuzumab, coltuximab, conatumumab, concizumab, cosfroviximab, CR6261, crenezumab, crizanlizumab (ADAKVEO®), crotedumab, cusatuzumab, dacetuzumab, daclizumab (ZINBRYTA®), dalotuzumab, dapirolizumab, daratumumab (DARZALEX®), dectrekumab, demcizumab, denintuzumab, denosumab (PROLIA®), depatuxizumab, derlotuximab, detumomab, dezamizumab, dinutuximab (UNITUXIN®), diridavumab, domagrozumab, dostarlimab, dorlimomab, dorlixizumab, drozitumab, DS-8201, duligotuzumab, dupilumab (DUPIXENT®), durvalumab (IMFINZI®), dusigitumab, ecromeximab, eculizumab (SOLRIS®), edobacomab, edrecolomab (PANOREX®), efalizumab (RAPTIVA®), efungumab (MYCOGRAB®), eldelumab, elezanumab, elgemtumab, elotuzumab (EMPLICITI®), elsilimomab, emactuzumab emapalumab (GAMIFANT®), emibetuzumab, emicizumab (HEMLIBRA®), enapotamab, enavatuzumab, enfortumab (PADCEV®), enlimomab, enoblituzumab, enokizumab, enoticumab, ensituximab, epitumomab, eptinezumab (VYEPTI®), epratuzumab, erenumab (AIMOVIG®), erlizumab, ertumaxomab (REXOMUN®), etaracizumab (ABEGRIN®), etigilimab, etrolizumab, evinacumab, evolocumab (REPATHA®), exbivirumab, fanolesomab (NEUTROSPEC®), faralimomab, faricimab, farletuzumab, fasinumab, FBTA05, felvizumab, fezakinumab, fibatuzumab, ficlatuzumab, figitumumab, firivumab, flanvotumab, fletikumab, flotetuzumab, fontolizumab (HUZAF®), foralumab, foravirumab, fremanezumab (AJOVY®), fresolimumab, frovocimab, frunevetmab, fulranumab, futuximab, galcanezumab (EMGALITY®), galiximab, gancotamab, ganitumab, gantenerumab, gavilimomab, gedivumab, gemtuzumab, gevokizumab, gilvetmab, gimsilumab, girentuximab, glembatumumab, golimumab (SIMPONI®), gomiliximab, guselkumab (TREMFYA®), huMy9-6, huMY9-6-IgG4-S228P, ianalumab, ibalizumab (TROGARZO®), IBI308, ibritumomab, icrucumab, idarucizumab (PRAXBIND®), ifabotuzumab, igovomab (INDIMACIS-125), iladatuzumab, IMAB362, imalumab, imaprelimab, imeiromab (MYOSCINT®), imgatuzumab, inclacumab, indatuximab, indusatumab, inebilizumab, infliximab (REMICADE®), intetumumab, inolimomab, inotuzumab, iomab-B, ipilimumab, iratumumab, isatuximab (SARCLISA®), iscalimab, istiratumab, itolizumab, ixekizumab (TALTZ®), keliximab, labetuzumab (CEA-CIDE™), lacnotuzumab, ladiratuzumab, lampalizumab, lanadelumab (TAKHZYRO®), landogrozumab, laprituximab, larcaviximab, lebrikizumab, lemalesomab, lendalizumab, lenvervimab, lenzilumab, lerdelimumab, leronlimab, lesofavumab, letolizumab, lexatumumab, libivirumab, lifastuzumab, ligelizumab, lilotomab, lintuzumab, lirilumab, lodelcizumab, lokivetmab, loncastuximab, lorvotuzumab, losatuxizumab, lucatumumab, lulizumab, lumiliximab, lumretuzumab, lupartumab, lutikizumab, mapatumumab, margetuximab, marstacimab, maslimomab, matuzumab, mavrilimumab, mepolizumab (NUCALA®), metelimumab, milatuzumab, minretumomab, mirikizumab, mirvetuximab, mitumomab, modotuximab, molalizumab, mogamulizumab (POTELIGEO®), morolimumab, mosunetuzumab, motavizumab (NUMAX®), moxetumomab (LUMOXITI®), muromonab-CD3 (ORTHOCLONE OKT3®), nacolomab, namilumab, naptumomab, naratuximab, narnatumab, natalizumab (TYSABRI®), navicixizumab, navivumab, naxitamab, nebacumab, necitumumab (PORTRAZZA®), nemolizumab, NEOD001, nerelimomab, nesvacumab, netakimab, nimotuzumab (THERACIM®), nirsevimab, nivolumab, nofetumomab, obiltoxaximab (ANTHIM®), obinutuzumab, ocaratuzumab, ocrelizumab (OCREVUS®), odulimomab, ofatumumab (ARZERRA®), olaratumab (LARTRUVO®), oleclumab, olendalizumab, olokizumab, omalizumab (XOLAIR®), omburtamab, OMS721, onartuzumab, ontecizumab, ontuxizumab, onvatilimab, opicinumab, oportuzumab, oregovomab (OVAREX), orticumab, otelixizumab, otilimab, otlertuzumab, oxelumab, ozanezumab, ozogamicin, ozoralizumab, pagibaximab, palivizumab (SYNAGIS®), pamrevlumab, panitumumab (VECTIBIX®), pankomab, panobacumab, parsatuzumab, pascolizumab, pasotuxizumab, pateclizumab, patritumab, PDR001, pembrolizumab, pemtumomab (THERAGYN®), perakizumab, pertuzumab (OMNITARG®), pexelizumab, pidilizumab, pinatuzumab, pintumomab, placulumab, polatuzumab (Polivy), prezalumab, plozalizumab, pogalizumab, ponezumab, porgaviximab, prasinezumab, prezalizumab, priliximab, pritoxaximab, pritumumab, PRO 140, quilizumab, racotumomab, radretumab, rafivirumab, ralpancizumab, ramucirumab, ranevetmab, ranibizumab (LUCENTIS®), ravagalimab, ravulizumab (ULTOMIRIS®), raxibacumab, refanezumab, regavirumab, REGN-EB3, renatlimab, remtolumab, reslizumab (CINQAIR®), rilotumumab, rinucumab, risankizumab (SKYRIZI®), rituximab (RITUXAN®), rivabazumab, rmab, robatumumab, roledumab, romilkimab, romosozumab (EVENITY®), rontalizumab, rosmantuzumab, rovalpituzumab, rovelizumab (LEUKARREST®), rozanolixizumab, ruplizumab (ANTOVA), SA237, sacituzumab, samalizumab, samrotamab, sarilumab (KEVZARA®), satralizumab, satumomab pendetide, secukinumab (COSENTYX®), selicrelumab, seribantumab, setoxaximab, setrusumab, sevirumab, SGN-CD19A, SHP647, sibrotuzumab, sifalimumab, siltuximab, simtuzumab, siplizumab, sirtratumab, sirukumab, sofituzumab, solanezumab, solitomab, sonepcizumab, sontuzumab, spartalizumab, stamulumab, STI-6129, sulesomab (LEUKOSCAN®), suptavumab, sutimlimab, suvizumab, suvratoxumab, tabalumab, tacatuzumab (AFP-CIDE®), tadocizumab, talacotuzumab, talizumab, tamtuvetmab, tanezumab, taplitumomab paptox, tarextumab, tavolimab, tefibazumab (AUREXIS®), telimomab, telisotuzumab, tesidolumab, tetraxetan, tetulomab, tenatumomab, teneliximab, teprotumumab (TEPEZZA®), teplizumab, tezepelumab, TGN1412, tibulizumab, ticilimumab (TREMELIMUMAB®), tigatuzumab, timigutuzumab, timolumab, tiragolumab, tiragotumab, tislelizumab, tisotumab, tiuxetan, tildrakizumab (ILUMYA®), TNX-650, tocilizumab (atlizumab, ACTEMRA®), tomuzotuximab, toralizumab, tosatoxumab, tositumomab (BEXXAR®), tovetumab, tralokinumab, trastuzumab (HERCEPTIN®), TRBS07, tregalizumab, tremelimumab, trevogrumab, tucotuzumab, tuvirumab, urtoxazumab, ustekinumab (STELERA®), ublituximab, ulocuplumab, urelumab, utomilumab, vadastuximab, vanalimab, vandortuzumab, vantictumab, vanucizumab, vapaliximab, varisacumab, varlilumab, vatelizumab, vedolizumab, veltuzumab, vepalimomab, vesencumab, visilizumab (NUVION®), vobarilizumab, volociximab (HUMASPECT®), vonlerolizumab, vopratelimab, vorsetuzumab, votumumab, vunakizumab, xentuzumab, XMAB-5574, zalutumumab (HuMEX-EGFr), zanolimumab (HuMAX-CD4), zatuximab, zenocutuzumab, ziralimumab, zolbetuximab or zolimomab.

In some aspects, the binding moiety useful for the present disclosure comprises an anti-CD33 antibody or antigen-binding portion thereof. CD33 is expressed in approximately 90% of acute myeloid leukemia (AML) cases and has demonstrated utility as a target of therapeutic antibodies. High CD33 expression on AML blasts has been reported approximately three decades ago. CD33 was detected on blasts of 85-90% of patients presenting with AML as well as on normal myeloid progenitors and myelocytes. CD33 is restricted to hematopoietic cells, but absent on normal hematopeoietic stem cells, making it an ideal target for AML therapy.

Anti-CD33 antibodies for the conjugates of the present disclosure are capable of specifically binding to CD33. In some aspects, anti-CD33 antibodies described herein bind to human CD33 with high affinity, for example, with a KD of 10⁻⁶ M or less, 10⁻⁷ M or less, 10⁻⁸ M or less, 10⁻⁹ M or less, 10⁻¹⁰ M or less, 10⁻¹¹ M or less, 10⁻¹²M or less, 10⁻¹²M to 10⁻⁷ M, 10⁻¹¹M to 10⁻⁷M, 10⁻¹⁰ M to 10⁻⁷ M, or 10⁻⁹ M to 10⁻⁷ M.

In some aspects, the anti-CD33 antibody comprises a heavy chain and a light chain, wherein the heavy chain comprises a heavy chain variable region (VH) and the light chain comprises a light chain variable region (VL); wherein the VH comprises a VH complementarity determining region (CDR) 1 (VH-CDR1), a VH-CDR2, and a VH-CDR3 and the VL comprises a VL-CDR1, a VL-CDR2, and a VL-CDR3; wherein the VH-CDR3 comprises an amino acid sequence having at least about 90%, at least about 95%, at least about 96%, at least about 97%, at least about 98%, or at least about 99% sequence identity to SEQ ID NO: 3. In some aspects, the anti-CD33 antibody comprises a VH-CDR2 comprising an amino acid sequence with at least about 90%, at least about 95%, at least about 96%, at least about 97%, at least about 98%, or at least about 99% sequence identity to an amino acid sequence of SEQ ID NO: 2. In some aspects, the anti-CD33 antibody comprises a VH-CDR1 comprising an amino acid sequence with at least about 90%, at least about 95%, at least about 96%, at least about 97%, at least about 98%, or at least about 99% sequence identity to an amino acid sequence of SEQ ID NO: 1. In some aspects, the anti-CD33 antibody comprises a VL-CDR1 comprising an amino acid sequence with at least about 90%, at least about 95%, at least about 96%, at least about 97%, at least about 98%, or at least about 99% sequence identity to an amino acid sequence of SEQ ID NO: 5. In some aspects, the anti-CD33 antibody comprises a VL-CDR2 comprising an amino acid sequence with at least about 90%, at least about 95%, at least about 96%, at least about 97%, at least about 98%, or at least about 99% sequence identity to an amino acid sequence of SEQ ID NO: 6. In some aspects, the anti-CD33 antibody comprises a VL-CDR3 comprising an amino acid sequence with at least about 90%, at least about 95%, at least about 96%, at least about 97%, at least about 98%, or at least about 99% sequence identity to an amino acid sequence of SEQ ID NO: 7. In some aspects, the CDRs comprises the sequences shown in Table 1 below.

TABLE 1
CDR Sequences and Variable Region Sequences

| | |
|---|---|
| VH-CDR1 | DSNIH |
| (SEQ ID NO: | |
| 1) | |
| VH-CDR2 | YIYPYNGGTDYNQKFKN |
| (SEQ ID NO: | |
| 2) | |
| VH-CDR3 | GNPWLAY |
| (SEQ ID NO: | |
| 3) | |
| VH (SEQ ID | EVQLVQSGAEVKKPGSSVKVSCKASGYTITDSNIHWVRQAPGQSLEWIGYIY |
| NO: 4) | PYNGGTDYNQKFKNRATLTVDNPTNTAYMELSSLRSEDTAFYYCVNGNPWL |
| | AYWGQGTLVTVSS |
| VL-CDR1 | RASESLDNYGIRFLT |
| (SEQ ID NO: | |
| 5) | |
| VL-CDR2 | AASNQGSG |
| (SEQ ID NO: | |
| 6) | |
| VL-CDR3 | QQTKEVPWS |
| (SEQ ID NO: | |
| 7) | |
| VL (SEQ ID | DIQLTQSPSTLSASVGDRVTITCRASESLDNYGIRFLTWFQQKPGKAPKLLMY |
| NO: 8) | AASNQGSGVPSRFSGSGSGTEFTLTISSLQPDDFATYYCQQTKEVPWSFGQGT |
| | KVEVK |

In some aspects, the anti-CD33 antibody heavy chain variable region comprises an amino acid sequence with at least about 80%, at least about 85%, at least about 90%, at least about 9500 at least about 96%, at least about 9700 at least about 98%, or at least about 9900 sequence identity to an amino acid sequence of SEQ ID NO: 4. In some aspects, the anti-CD33 antibody light chain variable region comprises an amino acid sequence with at least about 80%, at least about 85%, at least about 90%, at least about 9500, at least about 9600, at least about 970%, at least about 98%, or at least about 9900 sequence identity to an amino acid sequence of SEQ ID NO: 8.

In some aspects, the anti-CD33 antibody comprises a heavy chain variable region comprising a sequence with at least about 80%, at least about 85%, at least about 90%, at least about 9500 at least about 96%, at least about 9700 at least about 98%, or at least about 9900 sequence identity to an amino acid sequence of SEQ ID NO: 4, and a light chain variable region comprising a sequence with at least about 8000, at least about 8500, at least about 9000, at least about 9500, at least about 9600, at least about 9700, at least about 9800, or at least about 9900 sequence identity to an amino acid sequence of SEQ TD NO: 8.

In some aspects, the anti-CD33 antibody heavy chain comprises an amino acid sequence having at least about 8000, at least about 8500, at least about 9000, at least about 9500, at least about 9600, at least about 9700, at least about 9800, or at least about 9900 sequence identity to SEQ TD NO: 9 or SEQ TD NO: 11. In some aspects, the anti-CD33 antibody comprises a light chain comprising an amino acid sequence having at least about 80%, at least about 8500, at least about 90%, at least about 95%, at least about 96%, at least about 97%, at least about 98%, or at least about 99% sequence identity to SEQ ID NO: 10 or SEQ TD NO: 12.

Table 2. Anti-CD33 Antibody Amino Acid Sequences.

TABLE 2
Anti-CD33 Antibody Amino Acid Sequences.

| | |
|---|---|
| CD33AB | EVQLVQSGAEVKKPGSSVKVSCKASGYTITDSNIHWVRQAPGQSLEWIGYIY |
| Heavy Chain | PYNGGTDYNQKFKNRATLTVDNPTNTAYMELSSLRSEDTAFYYCVNGNPWL |
| (SEQ ID NO: | AYWGQGTLVTVSSASTKGPSVFPLAPSSKSTSGGTAALGCLVKDYFPEPVTVS |
| 9) | WNSGALTSGVHTFPAVLQSSGLYSLSSVVTVPSSSLGTQTYICNVNHKPSNTK |
| | VDKKVEPKSCDKTHTCPPCPAPELLGGPSVFLFPPKPKDTLMISRTPEVTCVV |
| | VDVSHEDPEVKFNWYVDGVEVHNAKTKPREEQYASTYRVVSVLTVLHQDW |
| | LNGKEYKCKVSNKALPAPIEKTISKAKGQPREPQVYTLPPSRDELTKNQVSLT |
| | CLVKGFYPSDIAVEWESNGQPENNYKTTPPVLDSDGSFFLYSKLTVDKSRWQ |
| | QGNVFSCSVMHEALHNHYTQKSLSLSPGK |
| CD33AB | DIQLTQSPSTLSASVGDRVTITCRASESLDNYGIRFLTWFQQKPGKAPKLLMY |
| Light Chain | AASNQGSGVPSRFSGSGSGTEFTLTISSLQPDDFATYYCQQTKEVPWSFGQGT |
| (SEQ ID NO: | KVEVKRTVAAPSVFIFPPSDEQLKSGTASVVCLLNNFYPREAKVQWKVDNAL |
| 10) | QSGNSQESVTEQDSKDSTYSLSSTLTLSKADYEKHKVYACEVTHQGLSSPVT |
| | KSFNRGEC |
| Gemtuzumab | EVQLVQSGAEVKKPGSSVKVSCKASGYTITDSNIHWVRQAPGQSLEWIGYIY |
| Heavy Chain | PYNGGTDYNQKFKNRATLTVDNPTNTAYMELSSLRSEDTAFYYCVNGNPWL |
| (SEQ ID NO: | AYWGQGTLVTVSSASTKGPSVFPLAPCSRSTSESTAALGCLVKDYFPEPVTVS |
| 11) | WNSGALTSGVHTFPAVLQSSGLYSLSSVVTVPSSSLGTKTYTCNVDHKPSNT |
| | KVDKRVESKYGPPCPPCPAPEFLGGPSVFLFPPKPKDTLMISRTPEVTCVVVD |
| | VSQEDPEVQFNWYVDGVEVHNAKTKPREEQFNSTYRVVSVLTVLHQDWLN |
| | GKEYKCKVSNKGLPSSIEKTISKAKGQPREPQVYTLPPSQEEMTKNQVSLTCL |
| | VKGFYPSDIAVEWESNGQPENNYKTTPPVLDSDGSFFLYSRLTVDKSRWQEG |
| | NVFSCSVMHEALHNHYTQKSLSLSLGK |
| Gemtuzumab | DIQLTQSPSTLSASVGDRVTITCRASESLDNYGIRFLTWFQQKPGKAPKLLMY |
| Light Chain | AASNQGSGVPSRFSGSGSGTEFTLTISSLQPDDFATYYCQQTKEVPWSFGQGT |
| SEQ ID NO: | KVEVKRTVAAPSVFIFPPSDEQLKSGTASVVCLLNNFYPREAKVQWKVDNAL |
| 12) | QSGNSQESVTEQDSKDSTYSLSSTLTLSKADYEKHKVYACEVTHQGLSSPVT |
| | KSFNRGEC |

In some aspects, the anti-CD33 antibody comprises a heavy chain comprising an ammino acid sequence having at least about 8000, at least about 8500, at least about 9000, at least about 9500, at least about 9600, at least about 9700, at least about 9800, or at least about 9900 sequence identity to SEQ ID NO: 9 and a light chain comprising an amino acid sequence having at least about 800%, at least about 850%, at least about 90%, at least about 9500 at least about 96%, at least about 97%, at least about 98%, or at least about 99% sequence identity to SEQ ID NO: 10. The term “CD33AB” comprises a heavy chain as set forth in SEQ ID NO: 9 and a light chain as set forth in SEQ ID NO: 10.

In some aspects, the anti-CD33 antibody comprises a heavy chain comprising an amino acid sequence having at least about 80%, at least about 85%, at least about 90%, at least about 95%, at least about 96%, at least about 97%, at least about 98%, or at least about 99% sequence identity to SEQ ID NO: 11 and a light chain comprising an amino acid sequence having at least about 80%, at least about 85%, at least about 90%, at least about 95%, at least about 96%, at least about 97%, at least about 98%, or at least about 99% sequence identity to SEQ ID NO: 12.

In some aspects, the anti-CD33 antibody is disclosed in U.S. Pat. Nos. 5,585,089, 5,693,762, each of which are expressly incorporated herein by reference.

In some aspects, the anti-CD33 antibody comprises a heavy chain and a light chain, wherein the heavy chain comprises a heavy chain variable region (VH) and the light chain comprises a light chain variable region (VL); wherein the VH comprises a VH complementarity determining region (CDR) 1 (VH-CDR1), a VH-CDR2, and a VH-CDR3 and the VL comprises a VL-CDR1, a VL-CDR2, and a VL-CDR3; wherein the VH-CDR3 comprises an amino acid sequence having at least about 90%, at least about 95%, at least about 96%, at least about 97%, at least about 98%, or at least about 99% sequence identity to SEQ ID NO: 15. In some aspects, the anti-CD33 antibody comprises a VH-CDR2 comprising an amino acid sequence with at least about 90%, at least about 95%, at least about 96%, at least about 97%, at least about 98%, or at least about 99% sequence identity to an amino acid sequence of SEQ ID NO: 14. In some aspects, the anti-CD33 antibody comprises a VH-CDR1 comprising an amino acid sequence with at least about 90%, at least about 95%, at least about 96%, at least about 97%, at least about 98%, or at least about 99% sequence identity to an amino acid sequence of SEQ ID NO: 13. In some aspects, the anti-CD33 antibody comprises a VL-CDR1 comprising an amino acid sequence with at least about 90%, at least about 95%, at least about 96%, at least about 97%, at least about 98%, or at least about 99% sequence identity to an amino acid sequence of SEQ ID NO: 18. In some aspects, the anti-CD33 antibody comprises a VL-CDR2 comprising an amino acid sequence with at least about 90%, at least about 95%, at least about 96%, at least about 97%, at least about 98%, or at least about 99% sequence identity to an amino acid sequence of SEQ ID NO: 19. In some aspects, the anti-CD33 antibody comprises a VL-CDR3 comprising an amino acid sequence with at least about 90%, at least about 95%, at least about 96%, at least about 97%, at least about 98%, or at least about 9900 sequence identity to an amino acid sequence of SEQ ID NO: 20. In some aspects, the CDRs comprises the sequences shown in Table 3 below.

TABLE 3
huMy9-6 and huMy9-6-IgG4-S228P CDR Sequences and Variable Region Sequences

| | |
|---|---|
| VH-CDR1 | SYYIH |
| (SEQ ID NO: | |
| 13) | |
| VH-CDR2 | VIYPGNDDISYNQKFQG |
| (SEQ ID NO: | |
| 14) | |
| VH-CDR3 | EVRLRYFDV |
| (SEQ ID NO: | |
| 15) | |
| VH (SEQ ID | QVQLQQPGAEVVKPGASVKMSCKASGYTFTSYYIHWIKQTPGQGLEW |
| NO: 16) | VGVIYPGNDDISYNQKFQGKATLTADKSSTTAYMQLSSLTSEDSAVYY |
| | CAREVRLRYFDVWGQGTTVTVSS |
| VL-CDR1 | KSSQSVFFSSSQKNYLA |
| (SEQ ID NO: | |
| 17) | |
| VL-CDR2 | WASTRES |
| (SEQ ID NO: | |
| 18) | |
| VL-CDR3 | HQYLSSRT |
| (SEQ ID NO: | |
| 19) | |
| VL (SEQ ID | EIVLTQSPGSLAVSPGERVTMSCKSSQSVFFSSSQKNYLAWYQQIPGQS |
| NO: 20) | PRLLIYWASTRESGVPDRFTGSGSGTDFTLTISSVQPEDLAIYYCHQYLS |
| | SRTFGQGTKLEIK |

In some aspects, the anti-D33 antibody heavy chain variable region comprises an amino acid sequence with at least about 800%, at least about 8500, at least about 900%, at least about 9500 at least about 960%, at least about 9700 at least about 980%, or at least about 9900 sequence identity to an amino acid sequence of SEQ ID NO: 16. In some aspects, the anti-CD33 antibody light chain variable region comprises an amino acid sequence with at least about 800%, at least about 850%, at least about 900%, at least about 9500 at least about 960%, at least about 9700 at least about 980%, or at least about 9900 sequence identity to an amino acid sequence of SEQ TD NO: 20.

In some aspects, the anti-CD33 antibody comprises a heavy chain variable region comprising a sequence with at least about 8000, at least about 8500, at least about 9000, at least about 9500, at least about 9600, at least about 9700, at least about 9800, or at least about 9900 sequence identity to an amino acid sequence of SEQ ID NO: 16, and a light chain variable region comprising a sequence with at least about 8000, at least about 8500, at least about 9000, at least about 95%, at least about 96%, at least about 97%, at least about 98%, or at least about 99% sequence identity to an amino acid sequence of SEQ ID NO: 20.

In some aspects, the anti-CD33 antibody heavy chain comprises an amino acid sequence having at least about 80%, at least about 85%, at least about 90%, at least about 95%, at least about 96%, at least about 97%, at least about 98%, or at least about 99% sequence identity to SEQ ID NO: 21. In some aspects, the anti-CD33 antibody comprises a light chain comprising an amino acid sequence having at least about 80%, at least about 85%, at least about 90%, at least about 95%, at least about 96%, at least about 97%, at least about 98%, or at least about 99% sequence identity to SEQ ID NO: 10 or SEQ ID NO: 22.

TABLE 4
huMy9-6-IgG4-S228P Antibody Amino Acid Sequences.

| | |
|---|---|
| Heavy Chain | QVQLQQPGAEVVKPGASVKMSCKASGYTFTSYYIHWIKQTPGQGLEW |
| (SEQ ID NO: | VGVIYPGNDDISYNQKFQGKATLTADKSSTTAYMQLSSLTSEDSAVYY |
| 21) | CAREVRLRYFDVWGQGTTVTVSSASTKGPSVFPLAPCSRSTSESTAAL |
| | GCLVKDYFPEPVTVSWNSGALTSGVHTFPAVLQSSGLYSLSSVVTVPS |
| | SSLGTKTYTCNVDHKPSNTKVDKRVESKYGPPCPPCPAPEFLGGPSVFL |
| | FPPKPKDTLMISRTPEVTCVVVDVSQEDPEVQFNWYVDGVEVHNAKT |
| | KPREEQFNSTYRVVSVLTVLHQDWLNGKEYKCKVSNKGLPSSIEKTIS |
| | KAKGQPREPQVYTLPPSQEEMTKNQVSLTCLVKGFYPSDIAVEWESNG |
| | QPENNYKTTPPVLDSDGSFFLYSRLTVDKSRWQEGNVFSCSVMHEAL |
| | HNHYTQKSLSLSLGK |
| Light Chain | EIVLTQSPGSLAVSPGERVTMSCKSSQSVFFSSSQKNYLAWYQQIPGQS |
| (SEQ ID NO: | PRLLIYWASTRESGVPDRFTGSGSGTDFTLTISSVQPEDLAIYYCHQYLS |
| 22) | SRTFGQGTKLEIKRTVAAPSVFIFPPSDEQLKSGTASVVCLLNNFYPRE |
| | AKVQWKVDNALQSGNSQESVTEQDSKDSTYSLSSTLTLSKADYEKHK |
| | VYACEVTHQGLSSPVTKSFNRGEC |

In some aspects, the anti-CD33 antibody comprises a heavy chain comprising an amino acid sequence having at least about 80%, at least about 85%, at least about 90%, at least about 95%, at least about 96%, at least about 97%, at least about 98%, or at least about 99% sequence identity to SEQ ID NO: 21 and a light chain comprising an amino acid sequence having at least about 80%, at least about 85%, at least about 90%, at least about 95%, at least about 96%, at least about 97%, at least about 98%, or at least about 99% sequence identity to SEQ ID NO: 22. The anti-CD33 antibody comprises a heavy chain as set forth in SEQ ID NO: 21 and a light chain as set forth in SEQ ID NO: 22.

An antibody “which binds” a molecular target or an antigen of interest is one capable of binding that antigen with sufficient affinity such that the antibody is useful in targeting a cell expressing the antigen.

In the present disclosure, group “Bm” can be conjugated to more than one neoDegrader. In some aspects, “Bm” can be conjugated to from 1 to 10 neoDegraders. In some aspects, “Bm” can be conjugated to from 1 to 9 neoDegraders. In some aspects, “Bm” can be conjugated to from 1 to 8 neoDegraders. In some aspects, “Bm” can be conjugated to 1, 2, 3, 4, 5, 6, 7, 8, 9, or 10 neoDegraders. In some aspects, “Bm” can be conjugated to 7 or 8 neoDegraders. In some aspects, “Bm” is conjugated to 5 neoDegraders. In some aspects, “Bm” is conjugated to 6 neoDegraders. In some aspects, “Bm” is conjugated to 7 neoDegraders. In some aspects, “Bm” is conjugated to 8 neoDegraders. In some aspects, “Bm” is conjugated to 9 neoDegraders.

### IV. Compositions and Methods of Using

The conjugates and/or compounds described herein can be in the form of pharmaceutically or pharmaceutically acceptable salts. In some aspects, such salts are derived from inorganic or organic acids or bases.

Examples of suitable acid addition salts include acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Examples of suitable base addition salts include ammonium salts; alkali metal salts, such as sodium and potassium salts; alkaline earth metal salts, such as calcium and magnesium salts; salts with organic bases, such as dicyclohexylamine salts, N-methyl-D-glucamine; and salts with amino acids such as arginine, lysine, and the like.

For example, Berge lists the following FDA-approved commercially marketed salts: anions acetate, besylate (benzenesulfonate), benzoate, bicarbonate, bitartrate, bromide, calcium edetate (ethylenediaminetetraacetate), camsylate (camphorsulfonate), carbonate, chloride, citrate, dihydrochloride, edetate (ethylenediaminetetraacetate), edisylate (1,2-ethanedisulfonate), estolate (lauryl sulfate), esylate (ethanesulfonate), fumarate, gluceptate (glucoheptonate), gluconate, glutamate, glycollylarsanilate (glycollamidophenylarsonate), hexylresorcinate, hydrabamine (N,N′-di(dehydroabietyl)ethylenediamine), hydrobromide, hydrochloride, hydroxynaphthoate, iodide, isethionate (2-hydroxyethanesulfonate), lactate, lactobionate, malate, maleate, mandelate, mesylate (methanesulfonate), methylbromide, methylnitrate, methylsulfate, mucate, napsylate (2-naphthalenesulfonate), nitrate, pamoate (embonate), pantothenate, phosphate/diphosphate, polygalacturonate, salicylate, stearate, subacetate, succinate, sulfate, tannate, tartrate, teoclate (8-chlorotheophyllinate) and triethiodide; organic cations benzathine (N,N′-dibenzylethylenediamine), chloroprocaine, choline, diethanolamine, ethylenediamine, meglumine (N-methylglucamine) and procaine; and metallic cations aluminum, calcium, lithium, magnesium, potassium, sodium and zinc.

Berge additionally lists the following non-FDA-approved commercially marketed (outside the United States) salts: anions adipate, alginate, aminosalicylate, anhydromethylenecitrate, arecoline, aspartate, bisulfate, butylbromide, camphorate, digluconate, dihydrobromide, disuccinate, glycerophosphate, hemisulfate, hydrofluoride, hydroiodide, methylenebis(salicylate), napadisylate (1,5-naphthalenedisulfonate), oxalate, pectinate, persulfate, phenylethylbarbiturate, picrate, propionate, thiocyanate, tosylate and undecanoate; organic cations benethamine (N-benzylphenethylamine), clemizole (1-p-chlorobenzyl-2-pyrrolildine-1′-ylmethylbenzimidazole), diethylamine, piperazine and tromethamine (tris(hydroxymethyl)aminomethane); and metallic cations barium and bismuth.

Pharmaceutical compositions comprising the neoDegrader conjugates described herein may also comprise suitable carriers, excipients, and auxiliaries that may differ depending on the mode of administration.

In some aspects, the pharmaceutical compositions can be formulated as a suitable parenteral dosage form. Said formulations can be prepared by various methods known in the art. The pharmaceutical compositions can be administered directly into the bloodstream, into muscle, or directly into an organ. Suitable means for parenteral administration include intravenous, intraarterial, intraperitoneal, intrathecal, intraventricular, intraurethral, intrasternal, intracranial, intramuscular, and subcutaneous. Suitable devices for parenteral administration include needle injectors, needle-free injectors, and infusion techniques.

Parenteral compositions are typically aqueous solutions which may contain excipients such as salts, carbohydrates and buffering agents. However, the composition may also be formulated a sterile non-aqueous solution or as a dried form to be used in conjunction with a suitable vehicle such as sterile pyrogen-free water.

The preparation of parenteral compositions under sterile conditions, for example, by lyophilization, can be readily accomplished using standard techniques known well to those of skill in the art.

Compositions for parenteral administration can be formulated to be immediate and/or modified release. Modified release formulations include delayed-, sustained-, pulsed-, controlled-, targeted, and programmed release. Thus, the compositions can be formulated as a solid, semi-solid, or thixotropic liquid for administration as an implanted depot providing modified release of the active agent.

The parenteral formulations can be admixed with other suitable pharmaceutically acceptable excipients used in parenteral dosage forms such as, but not limited to, preservatives.

In another aspect, the pharmaceutical compositions can be formulated as suitable oral dosage forms such as tablets, capsules, powders, pellets, suspensions, solutions, emulsions, and the like. Other suitable carriers can be present such as disintegrants, diluents, chelating agents, binders, glidants, lubricants, fillers, bulking agents, anti-adherants, and the like.

Oral dosage formulations may also contain other suitable pharmaceutical excipients such as sweeteners, vehicle/wetting agents, coloring agents, flavoring agents, preservatives, viscosity enhancing/thickening agents, and the like.

The neoDegrader conjugates described herein can be used to treat various cancers. Certain conjugates of the present disclosure can be superior in terms of efficacy expression, pharmacokinetics (e.g., absorption, distribution, metabolism, excretion), solubility (e.g., water solubility), interaction with other medicaments (e.g., drug-metabolizing enzyme inhibitory action), safety (e.g., acute toxicity, chronic toxicity, genetic toxicity, reproductive toxicity, cardiotoxicity, carcinogenicity, central toxicity) and/or stability (e.g., chemical stability, stability to an enzyme), and can be useful as a medicament.

The neoDegrader conjugates of the present disclosure can be used as medicaments such as an agents for the prophylaxis or treatment of diseases, for example, cancers e.g., colorectal cancers (e.g., colorectal cancer, rectal cancer, anus cancer, familial colorectal cancer, hereditary nonpolyposis colorectal cancer, gastrointestinal stromal tumor), lung cancers (e.g., non-small-cell lung cancer, small-cell lung cancer, malignant mesothelioma), mesothelioma, pancreatic cancers (e.g., pancreatic ductal carcinoma, pancreatic endocrine tumor), pharynx cancer, larynx cancer, esophageal cancer, stomach/gastric cancers (e.g., papillary adenocarcinoma, mucinous adenocarcinoma, adenosquamous carcinoma), duodenal cancer, small intestinal cancer, breast cancers (e.g., invasive ductal carcinoma, non-invasive ductal carcinoma, inflammatory breast cancer), ovarian cancers (e.g., ovarian epithelial cancer, extragonadal germ cell tumor, ovarian germ cell tumor, ovarian low-malignant potential tumor), testis tumor, prostate cancers (e.g., hormone-dependent prostate cancer, non-hormone dependent prostate cancer, castration-resistant prostate cancer), liver cancers (e.g., hepatocellular cancer, primary liver cancer, extrahepatic bile duct cancer), thyroid cancers (e.g., medullary thyroid carcinoma), renal cancers (e.g., renal cell cancers (e.g., clear cell renal cell cancer), transitional cell cancer of renal pelvis and ureter), uterine cancers (e.g., cervical cancer, uterine body cancer, uterus sarcoma), gestational choriocarcinoma, brain tumors (e.g., medulloblastoma, glioma, pineal astrocytic tumors, pilocytic astrocytoma, diffuse astrocytoma, anaplastic astrocytoma, pituitary adenoma), retinoblastoma, skin cancers (e.g., basalioma, malignant melanoma), sarcomas (e.g., rhabdomyosarcoma, leiomyosarcoma, soft tissue sarcoma, spindle cell sarcoma), malignant bone tumor, bladder cancer, hematological/blood cancers (e.g., multiple myeloma, leukemias (e.g., acute myelogenous leukemia), malignant lymphoma, Hodgkin's disease, chronic myeloproliferative disease), cancer of unknown primary; a cancer growth inhibitor; a cancer metastasis inhibitor; an apoptosis promoter; an agent for the treatment of precancerous lesions (e.g., myelodysplastic syndromes); and the like.

In certain aspects, neoDegrader conjugates of the present disclosure can be used as a medicament for the treatment of breast cancer, gastric cancer, ovarian cancer, uterine cancer, lung cancer, pancreatic cancer, liver cancer, lymphoma, or hematological cancers.

Furthermore, neoDegrader conjugates of the present disclosure can be used concurrently with, before or after a non-drug therapy. To be precise, the conjugates can be combined with a non-drug therapy such as (1) surgery, (2) hypertensive chemotherapy using angiotensin II etc., (3) gene therapy, (4) thermotherapy, (5) cryotherapy, (6) laser cauterization and (7) radiotherapy.

For example, by using a neoDegrader conjugate of the present disclosure before or after the above-mentioned surgery and the like, effects such as prevention of emergence of resistance, prolongation of Disease-Free Survival, suppression of cancer metastasis or recurrence, prolongation of life and the like may be afforded.

In addition, it is possible to combine a treatment with neoDegrader conjugates of the present disclosure with a supportive therapy: (i) administration of antibiotic (e.g., β-lactam type such as pansporin and the like, macrolide type such as clarithromycin and the like) for the complication with various infectious diseases, (ii) administration of high-calorie transfusion, amino acid preparation or general vitamin preparation for the improvement of malnutrition, (iii) administration of morphine for pain mitigation, (iv) administration of a pharmaceutical agent for ameliorating side effects such as nausea, vomiting, anorexia, diarrhea, leucopenia, thrombocytopenia, decreased hemoglobin concentration, hair loss, hepatopathy, renopathy, DIC, fever and the like and (v) administration of a pharmaceutical agent for suppressing multiple drug resistance of cancer and the like.

In some aspects, the neoDegrader or neoDegrader conjugate of the disclosure can be used in combination with a standard of care therapy, e.g., one or more therapeutic agents (e.g., anti-cancer agents and/or immunomodulating agents). Accordingly, in certain aspects, a method of treating a tumor disclosed herein comprises administering the neoDegrader or neoDegrader conjugate of the disclosure in combination with one or more additional therapeutic agents. In some aspects, the neoDegrader or neoDegrader conjugate of the disclosure can be used in combination with one or more anti-cancer agents, for example, such that multiple elements of the immune pathway can be targeted. In some aspects, an anti-cancer agent comprises an immune checkpoint inhibitor (i.e., blocks signaling through the particular immune checkpoint pathway). Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof.

In some aspects, the neoDegrader or neoDegrader conjugate of the disclosure is administered to the subject prior to or after the administration of the additional therapeutic agent. In other aspects, the neoDegrader or neoDegrader conjugate of the disclosure is administered to the subject concurrently with the additional therapeutic agent. In certain aspects, the neoDegrader or neoDegrader conjugate of the disclosure and the additional therapeutic agent can be administered concurrently as a single composition in a pharmaceutically acceptable carrier. In other aspects, the neoDegrader or neoDegrader conjugate of the disclosure and the additional therapeutic agent are administered concurrently as separate compositions.

In some aspects, a subject that can be treated with the neoDegrader or neoDegrader conjugate of the present disclosure is a nonhuman animal such as a rat or a mouse. In some aspects, the subject that can be treated is a human.

### V. Methods of Preparing NeoDegraders and Compositions

The present disclosure provides a method of preparing the neoDegrader conjugates, the process comprising reacting a binding moiety with a compound of formula (I-1):

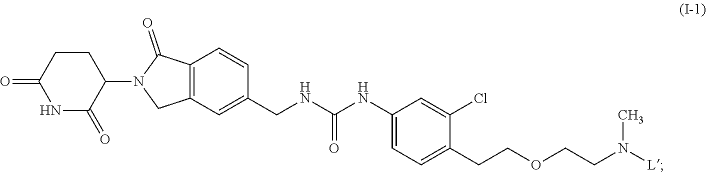

or a pharmaceutically acceptable salt thereof, wherein:
   L′ is a linker precursor selected from

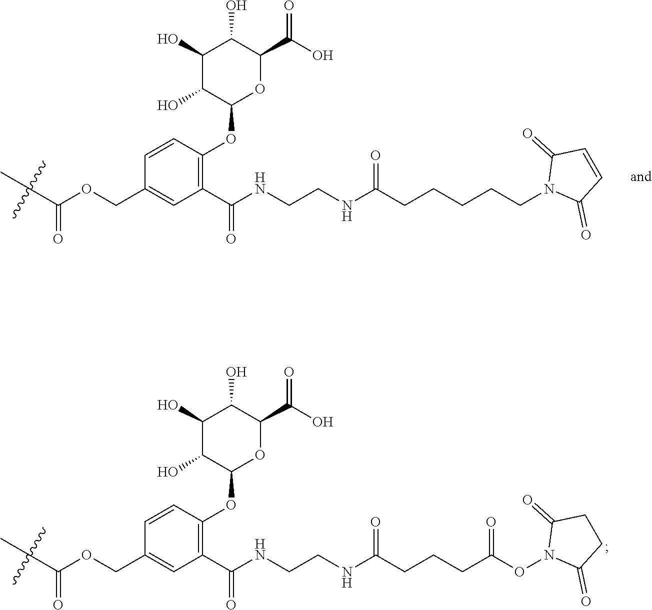 [SMILES: *C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1 ; *C(=O)OCc1ccc(O[C@@H]2O[C@H](C(=O)O)[C@@H](O)[C@H](O)[C@H]2O)c(C(=O)NCCNC(=O)CCCC(=O)ON2C(=O)CCC2=O)c1]

wherein:
   [symbol] is the point of attachment to the nitrogen atom.

As described herein, the linker precursors contain a heterobifunctional group that connects to the binding moiety.

In some aspects, the binding moiety is pre-treated before it is reacted with the compound of formula (I-1). In certain aspects, the compound of formula (I-1) is reacted with a binding moiety, which comprises an antibody or an antigen binding portion thereof. In aspects where the binding moiety is an antibody, the antibody can be pretreated to reduce interchain disulfides prior to reaction with the compound of formula (I-1).

### EXAMPLES

### General Synthetic Methods and Intermediates

The compounds of the present disclosure can be prepared by one of ordinary skill in the art in light of the present disclosure and knowledge in the art, and/or by reference to the schemes shown below and the synthetic examples. Exemplary synthetic routes are set forth in Schemes below and in Examples. It should be understood that the variables, (for example “R” groups) appearing in the following schemes and examples are to be read independently from those appearing elsewhere in the application. One of ordinary skill in the art would readily understand how the schemes and examples shown below illustrate the preparation of the compounds described herein.

Abbreviations used in the schemes generally follow conventions used in the art. Chemical abbreviations used in the specification and examples are defined as follows Abbreviations used in the schemes generally follow conventions used in the art. Chemical abbreviations used in the specification and examples are defined as follows: “Me” for methyl; “Bu” for butyl; “Ph” for phenyl; “TFA” for trifluoroacetic acid, “DCM” for dichloromethane “HATU” for N-[(dimethylamino)-1H-1,2,3-triazolo-[4,5-b]pyridin-1-ylmethylene]-N-methylmethanaminium hexafluorophosphate N-oxide; “TIHF” for tetrahydrofuran; “BOC” or “Boc” for tributoxycarbonyl; “TEA” for treithylamine; “EtOH” for ethanol; “DMF” for N,N-dimethylformamide; “PE” for petrolium ether; “EtOAc” for ethyl acetate; “DIEA” for diisopropylethylamine; “MeOH” for “methanol”; h for hours; min for minutes; “Ac” for acetate; “EDCI” for 1-ethyl-3-(3-dimethylaminopropyl)carbodiimide; “HOBT” for 1-hydroxybenzotriazole hydrate; “ACN” for acetonitrile; “TCEP” for (tris(2-carboxyethyl)phosphine); “DMA” for N,N-dimethyacetamide;

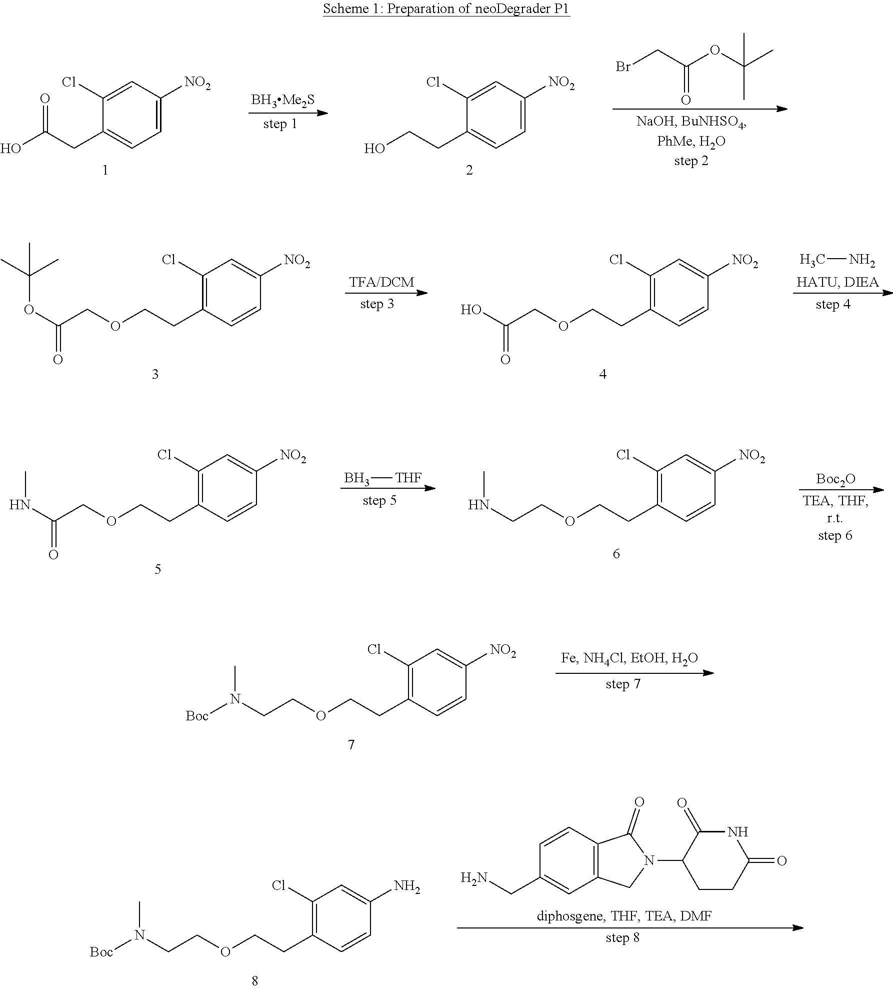

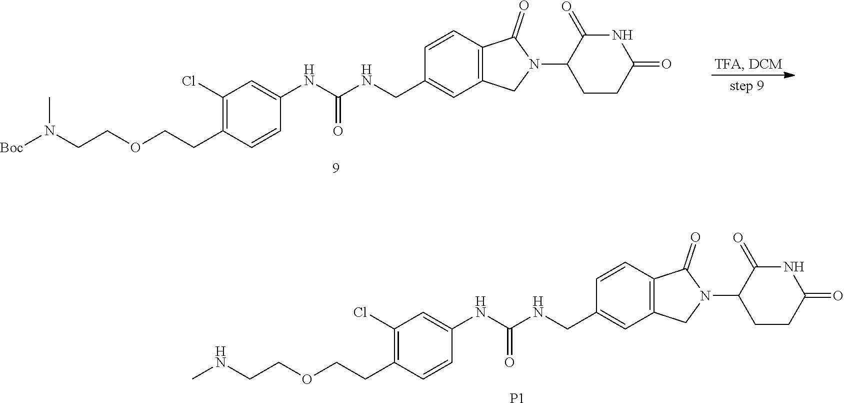

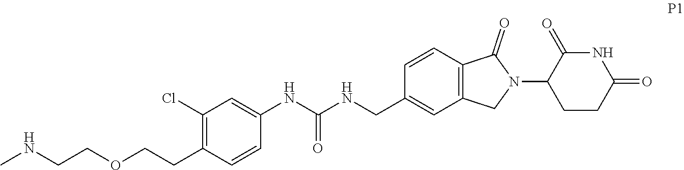 [SMILES: CNCCOCCc1ccc(NC(=O)NCc2ccc3c(c2)CN(C2CCC(=O)NC2=O)C3=O)cc1Cl]

### Example 1: Synthesis of NeoDegrader P1

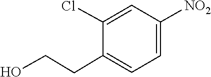 [SMILES: O=[N+]([O-])c1ccc(CCO)c(Cl)c1]

### Step 1: Synthesis of Compound 2

To a stirred solution of 2-chloro-4-nitrophenyl)acetic acid (Compound 1, 5.00 g, 23.19 mmol, 1.00 equiv) in THE (75.00 mL) was added BH₃-Me₂S (10M in THF) (5.80 mL, 58.0 mmol, 2.50 equiv) dropwise at 0° C. under nitrogen atmosphere. The resulting mixture was stirred for 2 h at 70° C. under nitrogen atmosphere. The mixture was cooled down to room temperature. The resulting mixture was concentrated under reduced pressure. The residue was purified by silica gel column chromatography (PE:EtOAc=1:1) to afford 2-(2-chloro-4-nitrophenyl)ethanol (3 g, 64%) as a yellow solid. ¹H NMR (400 MHz, CDCl₃) δ 8.26 (d, J=4.0 Hz, 1H), 8.10-8.05 (m, 1H), 7.50 (d, J=8.0 Hz, 1H), 3.99-3.91 (m, 2H), 3.16-3.09 (m, 2H).

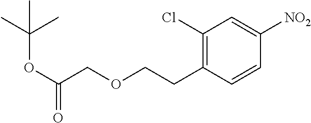 [SMILES: CC(C)(C)OC(=O)COCCc1ccc([N+](=O)[O-])cc1Cl]

### Step 2: Synthesis of Compound 3

To a stirred solution of 2-(2-chloro-4-nitrophenyl)ethanol (Compound 2, 5.00 g, 24.800 mmol, 1.00 equiv) and tert-butyl 2-bromoacetate (29.0 mL, 148.28 mmol, 8.00 equiv) in toluene (150.00 mL) was added Bu₄NHSO₄ (6.74 g, 19.84 mmol, 0.80 equiv). To the above mixture was added NaOH (5M in H₂O) (500.00 mL) dropwise over 40 min at 0° C. The resulting mixture was stirred for additional 2 h at 25° C. The resulting mixture was extracted with EtOAc (3×500 mL). The combined organic layers were washed with brine (400 mL) and dried over anhydrous Na₂SO₄. After filtration, the filtrate was concentrated under reduced pressure. The residue was purified by silica gel column chromatography (PE:EtOAc=4:1) to afford tert-butyl 2-[2-(2-chloro-4-nitrophenyl)ethoxy]acetate (8 g, 65%) as a yellow oil. ¹H NMR (400 MHz, CDCl₃) δ 8.23 (d, J=4.0 Hz, 1H), 8.10-8.04 (m, 1H), 7.60 (d, J=8.0 Hz, 1H), 4.09 (s, 2H), 3.83-3.80 (m, 2H), 3.17-3.14 (m, 2H), 1.45 (s, 9H).

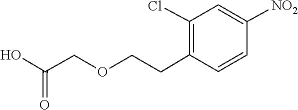 [SMILES: O=C(O)COCCc1ccc([N+](=O)[O-])cc1Cl]

### Step 3: Synthesis of Compound 4

To a stirred solution of tert-butyl 2-[2-(2-chloro-4-nitrophenyl)ethoxy]acetate (Compound 3, 8.00 g, 16.14 mmol, 1.00 equiv, 63.7%) in DCM (80.00 mL) was added TFA (16.00 mL) dropwise at room temperature. The resulting mixture was stirred for 1 h at room temperature. The resulting mixture was concentrated under vacuum. The resulting mixture was diluted with water (500 mL). The mixture was extracted with EtOAc (3×500 mL). The combined organic layers were washed with brine (200 mL), dried over anhydrous Na₂SO₄. After filtration, the filtrate was concentrated under reduced pressure. This resulted in [2-(2-chloro-4-nitrophenyl)ethoxy]acetic acid (6.5 g, crude) as yellow oil. LCMS (ESI): 517 (2M−H)−

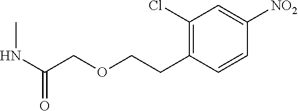 [SMILES: CNC(=O)COCCc1ccc([N+](=O)[O-])cc1Cl]

### Step 4: Synthesis of Compound 5

To a stirred solution of [2-(2-chloro-4-nitrophenyl)ethoxy]acetic acid (Compound 4, 6.30 g, 21.84 mmol, 1.00 equiv, 90%) and HATU (12.46 g, 32.76 mmol, 1.50 equiv) in DMF (65.00 mL) was added CH₃NH₂·HCl (1.77 g, 26.21 mmol, 1.20 equiv) and DIEA (15.20 g, 117.8 mmol, 4.00 equiv) dropwise at room temperature. The resulting mixture was stirred for 2 h at room temperature. The resulting mixture was diluted with water. The resulting mixture was extracted with EtOAc (2×100 mL). The combined organic layers were washed with brine (50 mL), dried over anhydrous Na₂SO₄. After filtration, the filtrate was concentrated under reduced pressure. The residue was purified by silica gel column chromatography (DCM:MeOH=10:1) to afford 2-[2-(2-chloro-4-nitrophenyl)ethoxy]-N-methylacetamide(10 g, purity:50%, yield:84%) as yellow oil. LCMS (ESI): 273.28 (M+H)⁺

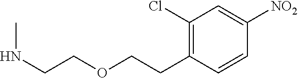 [SMILES: CNCCOCCc1ccc([N+](=O)[O-])cc1Cl]

### Step 5: Synthesis of Compound 6

To a stirred solution of 2-[2-(2-chloro-4-nitrophenyl)ethoxy]-N-methylacetamide (Compound 5, 3.3 g, 12.10 mmol, 1.00 equiv) in THF (35.00 mL) was added BH₃-THF (1M in THF) (12.10 mL, 12.10 mmol, 1.00 equiv) dropwise at room temperature under nitrogen atmosphere. The resulting mixture was stirred for 2 h at 70° C. under nitrogen atmosphere. The reaction was quenched with MeOH. The residue was acidified to pH 6 with 1N HCl. The resulting mixture was extracted with EtOAc (20 mL). The aqueous phase was basified to pH 8 with saturated NaHCO₃ (sat., aq.). The resulting mixture was extracted with EtOAc (3×100 mL), washed with brine (50 mL) and dried over anhydrous Na₂SO₄. After filtration, the filtrate was concentrated under reduced pressure. This resulted in [2-[2-(2-chloro-4-nitrophenyl)ethoxy]ethyl](methyl)amine (2.5 g, 80%) as yellow oil. LCMS (ESI): 259.26 (M+H)⁺

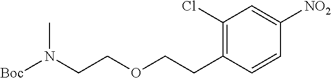 [SMILES: CN(CCOCCc1ccc([N+](=O)[O-])cc1Cl)C(=O)OC(C)(C)C]

### Step 6. Synthesis of Compound 7

To a stirred solution of [2-[2-(2-chloro-4-nitrophenyl)ethoxy]ethyl](methyl)amine (Compound 6, 2.50 g, 9.69 mmol, 1.00 equiv) and Boc₂O (2.53 g, 11.6 mmol, 1.20 equiv) in THF (40 mL) was added TEA (1.17 g, 11.6 mmol, 1.20 equiv) dropwise at 25° C. The mixture was stirred at 25° C. for 2 h. The resulting mixture was concentrated under vacuum. The residue was purified by silica gel column chromatography (DCM:MeOH=5:1) to afford tert-butyl N-[2-[2-(2-chloro-4-nitrophenyl)ethoxy]ethyl]-N-methylcarbamate(1.70 g, 50%) as yellow oil. LCMS (ESI): 359.36 (M+H)⁺

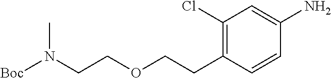 [SMILES: CN(CCOCCc1ccc(N)cc1Cl)C(=O)OC(C)(C)C]

### Step 7: Synthesis of Compound 8

To a stirred solution of tert-butyl N-[2-[2-(2-chloro-4-nitrophenyl)ethoxy]ethyl]-N-methylcarbamate (Compound 7, 1.70 g, 4.74 mmol, 1.00 equiv) and NH₄Cl (750 mg, 14.2 mmol, 3.00 equiv) in EtOH (85 mL) and H₂O (17 mL) was added Fe (1.3 g, 23.7 mmol, 5.00 equiv) at 25° C. The mixture was stirred at 80° C. for 2 h. The mixture was cooled down to room temperature. The resulting mixture was filtered, and the filter cake was washed with EtOH (3×50 mL). The filtrate was concentrated under reduced pressure. The residue was purified by silica gel column chromatography (PE EtOAc=4:1) to afford tert-butyl N-[2-[2-(4-amino-2-chlorophenyl)ethoxy]ethyl]-N-methylcarbamate (900 mg, 58%) as yellow oil. LCMS (ESI): 329.33 (M+H)⁺

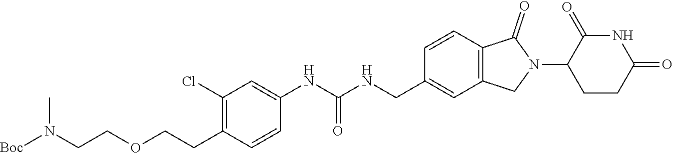 [SMILES: CN(CCOCCc1ccc(NC(=O)NCc2ccc3c(c2)CN(C2CCC(=O)NC2=O)C3=O)cc1Cl)C(=O)OC(C)(C)C]

### Step 8: Synthesis of Compound 9

To a stirred solution of tert-butyl N-[2-[2-(4-amino-2-chlorophenyl)ethoxy]ethyl]-N-methylcarbamate (Compound 8, 500 mg, 1.52 mmol, 1.00 equiv) in THE (10 mL) was added diphosgene (601 mg, 3.04 mmol, 2.00 equiv) dropwise at 25° C. The mixture was stirred at 25° C. for 1 h. The resulting mixture was concentrated under vacuum and re-dissolved in DMF (5 mL). To a stirred mixture of 3-[5-(aminomethyl)-1-oxo-3H-isoindol-2-yl]piperidine-2,6-dione (INT1, prepared as described below, 499 mg, 1.82 mmol, 1.20 equiv) and TEA (1.56 g, 15.45 mmol, 10.00 equiv) in DMF (20 mL) was added the solution mentioned above dropwise at 25° C. The mixture was stirred at 25° C. for 1 h. The resulting mixture was diluted with 40 mL of ice water. The resulting mixture was extracted with EtOAc (3×40 mL). The combined organic layers were washed with brine (5×40 mL) and dried over anhydrous Na₂SO₄. After filtration, the filtrate was concentrated under reduced pressure. The residue was purified by silica gel column chromatography (DCM:MeOH=10:1) to afford tert-butyl (2-(2-chloro-4-(3-((2-(2,6-dioxopiperidin-3-yl)-1-oxoisoindolin-5-yl)methyl)ureido)phenethoxy)ethyl)(methyl)carbamate (670 mg, 70%) as a white solid. LCMS: (ESI): 628.63 (M+H)⁺

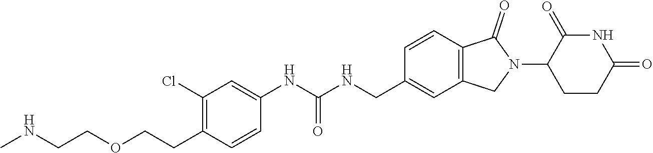 [SMILES: CNCCOCCc1ccc(NC(=O)NCc2ccc3c(c2)CN(C2CCC(=O)NC2=O)C3=O)cc1Cl]

### Step 9: Synthesis of neoDegrader P1

To a stirred solution of tert-butyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (Compound 9, 670 mg, 1.07 mmol, 1 eq) in DCM (10 mL) was added TFA (2.5 mL) dropwise at 0° C. The mixture was stirred at 25° C. for 1 h. The resulting mixture was concentrated under vacuum. The crude product was purified by Prep-HPLC with the following conditions: Column, SunFire C18 OBD Prep Column, 100 m, 19×250 mm; mobile phase, water (0.05% TFA) and ACN (5% Phase B up to 60% in 30 min); Detector, UV 220 nm. The collected fraction was lyophilized to give 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (500 mg, 89%) as a white solid. LCMS (ESI): 528.53 (M+H)⁺. ¹H NMR (400 MHz, Methanol-d4) δ 7.77 (d, J=8.0 Hz, 1H), 7.57-7.53 (m, 2H), 7.49 (d, J=8.0 Hz, 1H), 7.21 (d, J=4.0 Hz, 2H), 5.19-5.1 (m, 1H), 4.55-4.41 (m, 4H), 3.75-3.67 (m, 4H), 3.21-3.15 (m, 2H), 3.03-3.96 (m, 2H), 2.96-2.84 (m, 1H), 2.83-2.73 (m, 2H), 2.69 (s, 3H), 2.55-2.42 (m, 1H), 2.21-2.12 (m, 1H).

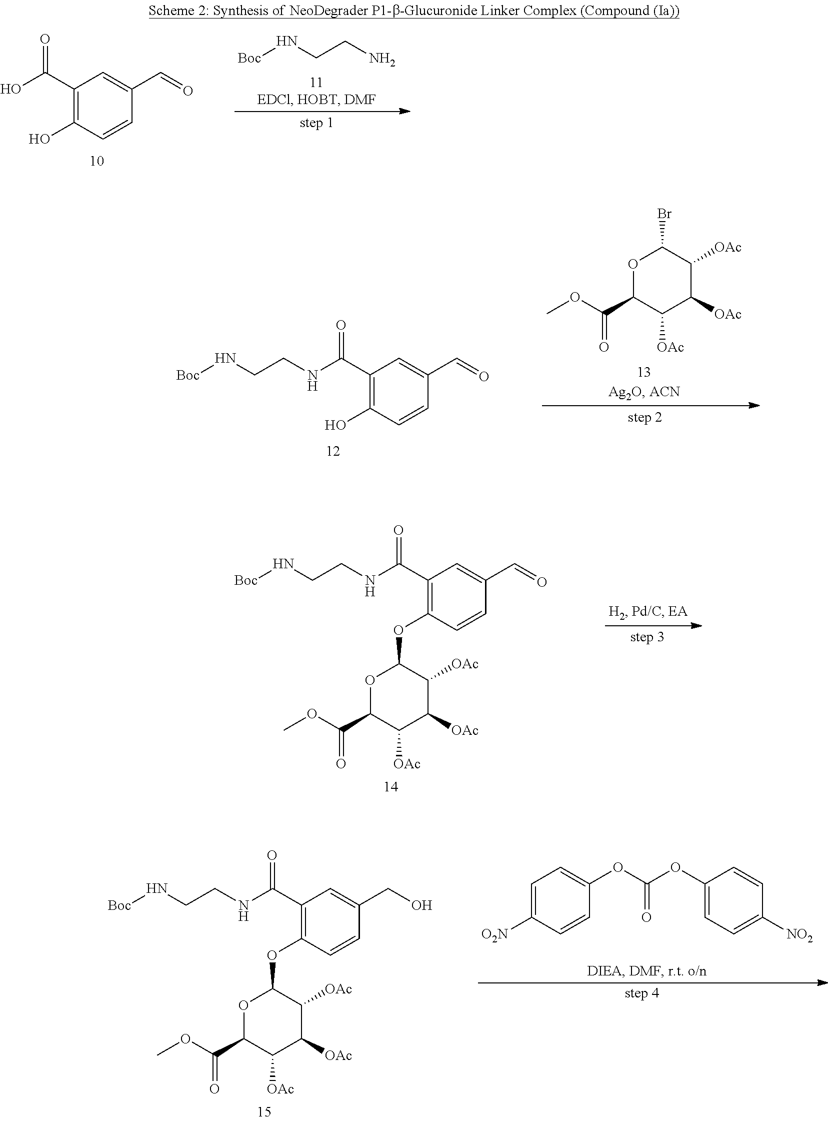

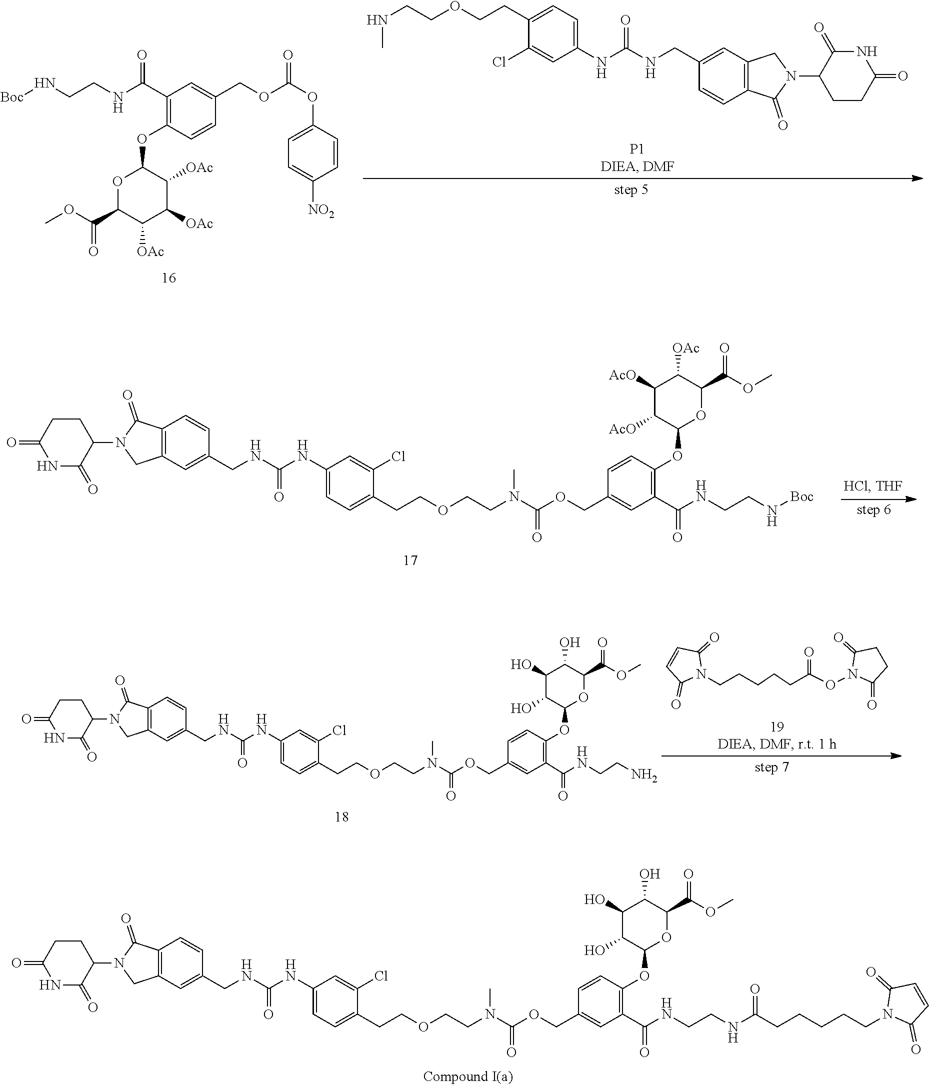

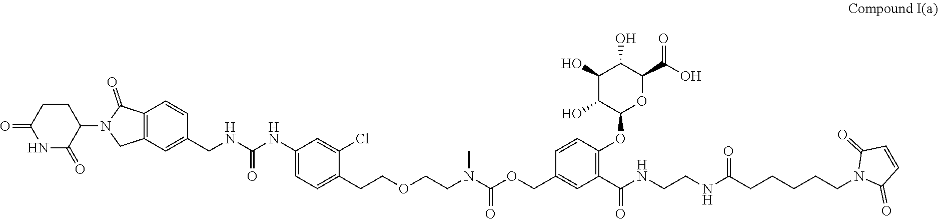 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

### Example 2: Synthesis of Compound (Ia)

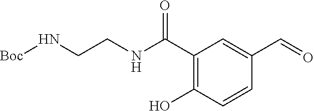 [SMILES: CC(C)(C)OC(=O)NCCNC(=O)c1cc(C=O)ccc1O]

### Step 1: Synthesis of Compound 12

To a stirred solution of 5-formyl-2-hydroxybenzoic acid, 10 (20 g, 120.38 mmol, 1.00 equiv) in DMF (200 mL) were added EDCI (28 g, 144.44 mmol, 1.20 equiv), HOBT (20 g, 144.46 mmol, 1.20 equiv) and tert-butyl N-(2-aminoethyl)carbamate, 11 (23 g, 144.46 mmol, 1.20 equiv) in portions at 0° C. under nitrogen atmosphere. The resulting mixture was stirred for overnight at room temperature under nitrogen atmosphere. LCMS indicated the reaction was completed. The reaction mixture was quenched with water and extracted with ethyl acetate (3×200 mL). The combined organic was washed with brine (200 mL), dried with Na₂SO₄. After filtration, the filtrate was concentrated to dryness under vacuum. The residue was purified by silica gel column chromatography, eluted with PE/EA (2:3) to afford tert-butyl N-[2-[(5-formyl-2-hydroxyphenyl)formamido]ethyl]carbamate, 12 (23 g, 53%) as a white solid. LCMS (ES, m/z): 209 [M+H−100]⁺, 309 [M+H]⁺, 331 [M+Na]⁺.

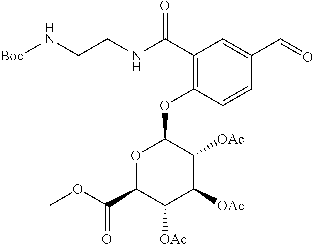 [SMILES: COC(=O)[C@H]1O[C@@H](Oc2ccc(C=O)cc2C(=O)NCCNC(=O)OC(C)(C)C)[C@H](OC(C)=O)[C@@H](OC(C)=O)[C@@H]1OC(C)=O]

### Step 2: Synthesis of Compound 14

To a stirred solution of tert-butyl N-[2-[(5-formyl-2-hydroxyphenyl)formamido]ethyl]carbamate, 12 (23 g, 74.59 mmol, 1.00 equiv) in ACN (600 mL) were added Ag₂O (34.57 g, 149.17 mmol, 2.00 equiv) and methyl (2S,3S,4S,5R,6R)-3,4,5-tris(acetyloxy)-6-bromooxane-2-carboxylate, 13 (32.6 g, 82.05 mmol, 1.10 equiv) in portions at 0° C. under nitrogen atmosphere. The resulting mixture was stirred for overnight at room temperature under nitrogen atmosphere. LCMS indicated the reaction was completed. After filtration, the filtrate was concentrated to dryness under vacuum. The residue was purified by silica gel column chromatography, eluted with PE/EA (1:4) to afford methyl (2S,3S,4S,5R,6S)-3,4,5-tris(acetyloxy)-6-[2-([2-[(tert-butoxycarbonyl)amino]ethyl]carbamoyl)-4-formylphenoxy]oxane-2-carboxylate, 14 (38 g, 76%) as a green solid. LCMS (ES, m/z): 525 [M+H−100]⁺, 625 [M+H]⁺, 647 [M+Na]⁺.

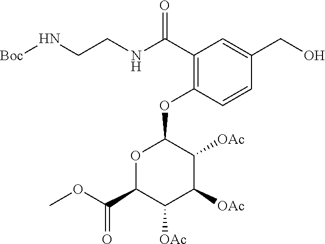 [SMILES: COC(=O)[C@H]1O[C@@H](Oc2ccc(CO)cc2C(=O)NCCNC(=O)OC(C)(C)C)[C@H](OC(C)=O)[C@@H](OC(C)=O)[C@@H]1OC(C)=O]

### Step 3: Synthesis of Compound 15

To a stirred solution of methyl (2S,3S,4S,5R,6S)-3,4,5-tris(acetyloxy)-6-[2-({2-[(tert-butoxycarbonyl)amino]ethyl}carbamoyl)-4-formylphenoxy]oxane-2-carboxylate, 14 (20 g, 32.02 mmol, 1.00 equiv) in EA (200 mL) were added Pd/C (4.0 g, 10%) in portions at room temperature. The resulting mixture was stirred overnight at room temperature under hydrogen atmosphere. LCMS indicated the reaction was completed. After filtration, the filtrate was concentrated to dryness under vacuum. The residue was purified by silica gel column chromatography, eluted with DCM/MeOH (19:1) to afford methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate, 15 (12.5 g, 58%) as a white solid. LCMS (ES, m/z): 527 [M+H−100]⁺, 627 [M+H]⁺, 649 [M+Na]⁺.

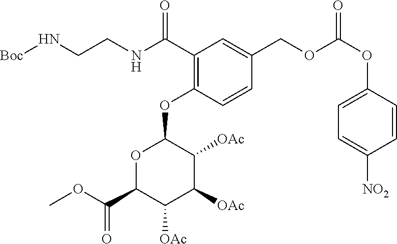 [SMILES: COC(=O)[C@H]1O[C@@H](Oc2ccc(COC(=O)Oc3ccc([N+](=O)[O-])cc3)cc2C(=O)NCCNC(=O)OC(C)(C)C)[C@H](OC(C)=O)[C@@H](OC(C)=O)[C@@H]1OC(C)=O]

### Step 4: Synthesis of Compound 16

To a stirred solution of methyl (2S,3S,4S,5R,6S)-3,4,5-tris(acetyloxy)-6-[2-({2-[(tert-butoxycarbonyl)amino]ethyl}carbamoyl)-4-(hydroxymethyl)phenoxy]oxane-2-carboxylate, 15 (5.0 g, 7.98 mmol, 1.00 equiv) in DMF (50 mL) were added bis(4-nitrophenyl) carbonate (2.67 g, 8.77 mmol, 1.10 equiv) and DIEA (2.0 g, 15.94 mmol, 2.00 equiv) in portions at 0° C. under nitrogen atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 70% gradient in 40 min; detector, UV 254 nm. The collected fraction was concentrated to dryness under vacuum to afford methyl (2S,3S,4S,5R,6S)-3,4,5-tris(acetyloxy)-6-[2-({2-[(tert-butoxycarbonyl)amino]ethyl}carbamoyl)-4-{[(4-nitrophenoxycarbonyl)oxy]methyl}phenoxy]oxane-2-carboxylate, 16 (5.3 g, 78%) as a white solid. LCMS (ES, m/z): 692 [M+H−100]⁺, 792 [M+H]⁺, 814 [M+Na]⁺.

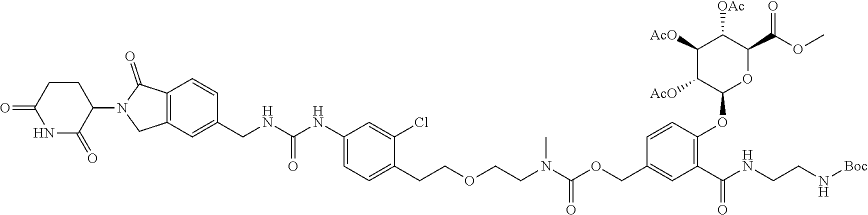 [SMILES: COC(=O)[C@H]1O[C@@H](Oc2ccc(COC(=O)N(C)CCOCCc3ccc(NC(=O)NCc4ccc5c(c4)CN(C4CCC(=O)NC4=O)C5=O)cc3Cl)cc2C(=O)NCCNC(=O)OC(C)(C)C)[C@H](OC(C)=O)[C@@H](OC(C)=O)[C@@H]1OC(C)=O]

### Step 5: Synthesis of Compound 17

To a stirred solution of methyl methyl (2S,3S,4S,5R,6S)-3,4,5-tris(acetyloxy)-6-[2-({2-[(tert-butoxycarbonyl)amino]ethyl}carbamoyl)-4-{[(4-nitrophenoxycarbonyl)oxy]methyl}phenoxy]oxane-2-carboxylate, 16 (550 mg, 0.69 mmol, 1.00 equiv) and 1-(3-chloro-4-{2-[2-(methylamino)ethoxy]ethyl}phenyl)-3-{[2-(2,6-dioxopiperidin-3-yl)-1-oxo-3H-isoindol-5-yl]methyl}urea, P1 (367 mg, 0.69 mmol, 1.00 equiv) in DMF (6.0 mL) were added DIEA (180 mg, 1.38 mmol, 2.00 equiv) in portions at room temperature under nitrogen atmosphere. The resulting mixture was stirred for 2 h at room temperature. LCMS indicated the reaction was completed. The reaction mixture was purified by reverse flash chromatography with the following conditions: column, C18 silica gel; mobile phase, ACN in water (0.1% FA), 10% to 70% gradient in 40 min; detector, UV 254 nm. The collected fraction was concentrated under vacuum to afford methyl (2S,3S,4S,5R,6S)-3,4,5-tris(acetyloxy)-6-[2-({2-[(tert-butoxycarbonyl)amino]ethyl}carbamoyl)-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy]oxa ne-2-carboxylate, 17 (670 mg, 77%) as a green solid. LCMS (ES, m/z): 1080 [M+H−100]⁺, 1180 [M+H]⁺, 1202 [M+Na]⁺.

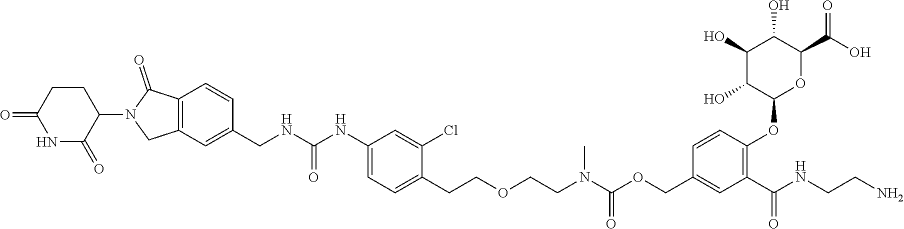 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCN)c1]

### Step 6: Synthesis of Compound 18

To a stirred solution of methyl (2S,3S,4S,5R,6S)-3,4,5-tris(acetyloxy)-6-[2-({2-[(tert-butoxycarbonyl)amino]ethyl}carbamoyl)-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy]oxa ne-2-carboxylate, 17 (660 mg, 0.56 mmol, 1.00 equiv) in THE (12 mL) was added HCl (12 mL, 6.0N) in portions at 45° C. under nitrogen atmosphere. The resulting mixture was stirred for 4 h at 45° C. under nitrogen atmosphere. LCMS indicated the reaction was completed. The reaction mixture was purified by reverse flash chromatography with the following conditions: column, C18 silica gel; mobile phase, ACN in water (0.1% FA), 10% to 40% gradient in 30 min; detector, UV 254 nm. The collected fraction was lyophilized to afford (2S,3S,4S,5R,6S)-6-{2-[(2-aminoethyl)carbamoyl]-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy}-3,4,5-trihydroxyoxane-2-carboxylic acid (320 mg, 54%), 18 as a white solid. LCMS (ES, m/z): 940 [M+H]⁺, 962 [M+Na]⁺.

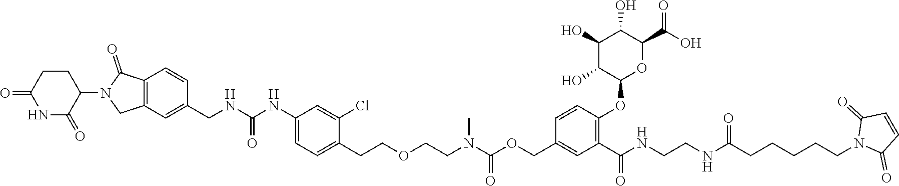 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

### Step 7: Synthesis of Compound (Ia)

To a stirred solution of (2S,3S,4S,5R,6S)-6-{2-[(2-aminoethyl)carbamoyl]-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy}-3,4,5-trihydroxyoxane-2-carboxylic acid, 18 (100 mg, 0.11 mmol, 1.00 equiv) and 2,5-dioxopyrrolidin-1-yl 6-(2,5-dioxopyrrol-1-yl)hexanoate, 19 (36 mg, 0.12 mmol, 1.10 equiv) in DMF (1.00 mL) was added DIEA (27 mg, 0.21 mmol, 2.0 equiv) in portions at room temperature under nitrogen atmosphere. The resulting mixture was stirred for 1 h at room temperature under nitrogen atmosphere. LCMS indicated the reaction was completed. The reaction mixture was purified by Prep-HPLC with the following conditions: Xselect CSH F-Phenyl OBD column, 19×250 mm; Mobile Phase A: Water (0.1% FA), Mobile Phase B: ACN; Flow rate: 25 mL/min; detector, UV 254 nm. The collected fraction was lyophilized to afford (2S,3S,4S,5R,6S)-6-{4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]-2-({2-[6-(2,5-dioxopyrrol-1-yl)hexanamido]ethyl}carbamoyl)phenoxy}-3,4,5-trihydroxyoxane-2-carboxylic acid, Compound (Ia) (46 mg, 37%) as a white solid. LCMS (ES, m/z): 568 [M/2+H]⁺, 1133 [M+H]⁺, 1155 [M+Na]⁺. ¹H-NMR (300 MHz, DMSO-d6): 10.98 (s, 1H), 8.87 (br s, 1H), 8.30 (t, J=6 Hz, 1H), 7.86 (t, J=3 Hz, 1H), 7.77 (s, 1H), 7.70-7.69 (m, 2H), 7.51-7.42 (m, 3H), 7.30-7.10 (m, 3H), 6.99 (s, 2H), 6.93 (br s, 1H), 5.79-5.77 (m, 1H), 5.36 (d, J=4.2 Hz, 1H), 5.13-5.02 (m, 4H), 4.47-4.28 (m, 4H), 3.95 (d, J=9.0 Hz, 1H), 3.540-3.49 (m, 4H), 3.39-3.36 (m, 4H), 3.24-3.22 (m, 2H), 2.86-2.81 (m, 7H), 2.62-2.51 (m, 1H), 2.49-2.41 (m, 3H), 2.06-1.99 (m, 3H), 1.49-1.42 (m, 4H), 1.23-1.16 (m, 2H).

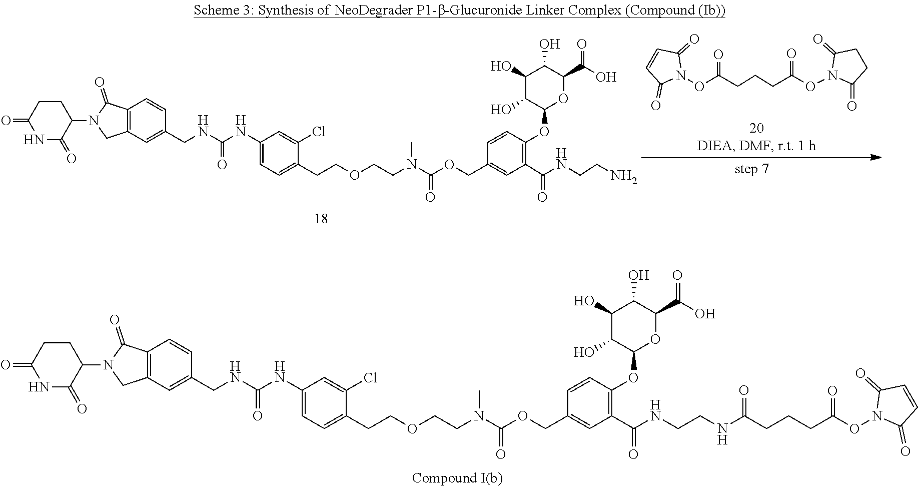 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCN)c1 ; O=C(CCCC(=O)ON1C(=O)CCC1=O)ON1C(=O)C=CC1=O ; CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCC(=O)ON2C(=O)C=CC2=O)c1]

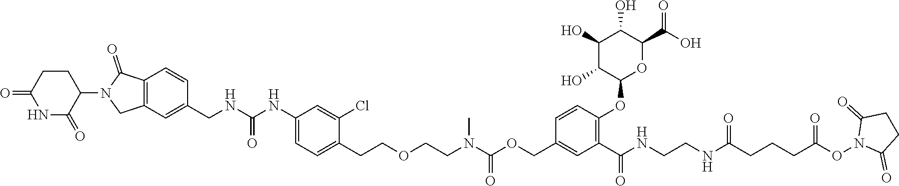 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCC(=O)ON2C(=O)CCC2=O)c1]

### Example 3: Synthesis of Compound (Ib)

To a stirred solution of bis(2,5-dioxopyrrolidin-1-yl) pentanedioate (62 mg, 0.19 mmol, 1.2 equiv) and (2S,3S,4S,5R,6S)-6-{2-[(2-aminoethyl)carbamoyl]-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy}-3,4,5-trihydroxyoxane-2-carboxylic acid, 9 (150 mg, 0.16 mmol, 1.00 equiv) in DMF (3.00 mL) was added DIEA (41 mg, 0.32 mmol, 2.00 equiv) in portions at room temperature under nitrogen atmosphere. The resulting mixture was stirred for 1 h at room temperature under nitrogen atmosphere. LCMS indicated the reaction was completed. The crude product was purified by Prep-HPLC with the following conditions (Column: Mobile Phase A: Water (0.05% TFA), Mobile Phase B: ACN; Flow rate: 60 mL/min;). The solution was lyophilized to afford (2S,3S,4S,5R,6S)-6-{4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]-2-[(2-{5-[(2,5-dioxopyrrolidin-1-yl)oxy]-5-oxopentanamido}ethyl)carbamoyl]phenoxy}-3,4,5-trihydroxyoxane-2-carboxylic acid, Compound (Ib) (50 mg, 26%) as a white solid. LCMS (ES, m/z): 576 [M/2+H]⁺, 1151 [M+H]⁺, 1173 [M+Na]⁺. ¹H-NMR (300 MHz, DMSO-d6): 12.90 (br s, 1H), 10.98 (s, 1H), 8.78 (s, 1H), 8.31 (t, J=3.0 Hz, 1H), 7.92 (t, J=3.0 Hz, 1H), 7.77 (d, J=3.0 Hz, 1H), 7.70-7.66 (m, 2H), 7.51-7.43 (m, 3H), 7.25-7.09 (m, 3H), 6.82 (t, J=3.0 Hz, 1H), 5.80-5.20 (m, 2H), 5.12-5.02 (m, 4H), 4.47-4.27 (m, 4H), 3.98 (d, J=9.3 Hz, 1H), 3.52-3.44 (m, 5H), 3.30-3.05 (m, 7H), 2.97-2.73 (m, 9H), 2.72-2.62 (m, 3H), 2.45-2.35 (m, 1H), 2.30-2.15 (m, 2H), 2.05-1.90 (m, 1H), 1.90-1.70 (m, 2H).

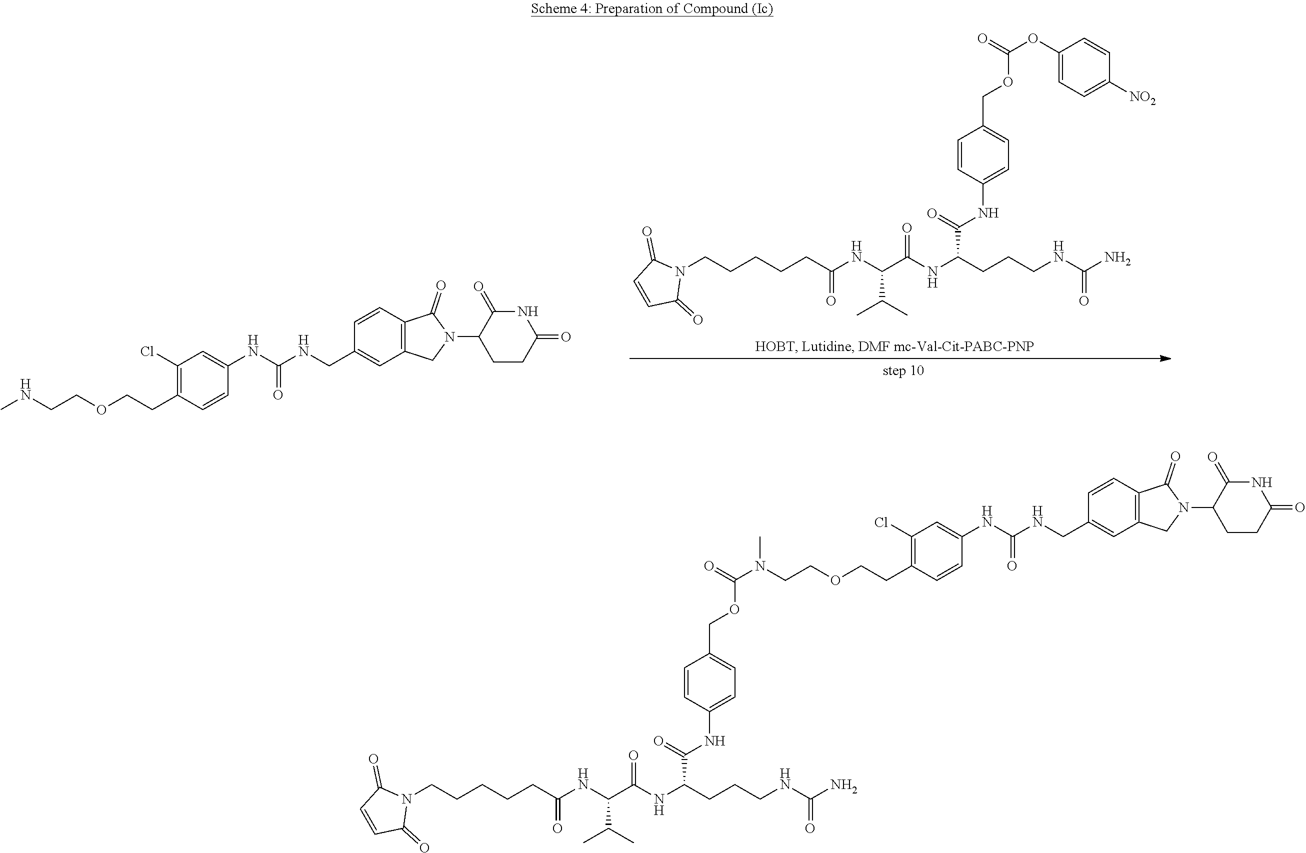 [SMILES: CNCCOCCc1ccc(NC(=O)NCc2ccc3c(c2)CN(C2CCC(=O)NC2=O)C3=O)cc1Cl ; CC(C)[C@H](NC(=O)CCCCCN1C(=O)C=CC1=O)C(=O)N[C@@H](CCCNC(N)=O)C(=O)Nc1ccc(COC(=O)Oc2ccc([N+](=O)[O-])cc2)cc1 ; CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

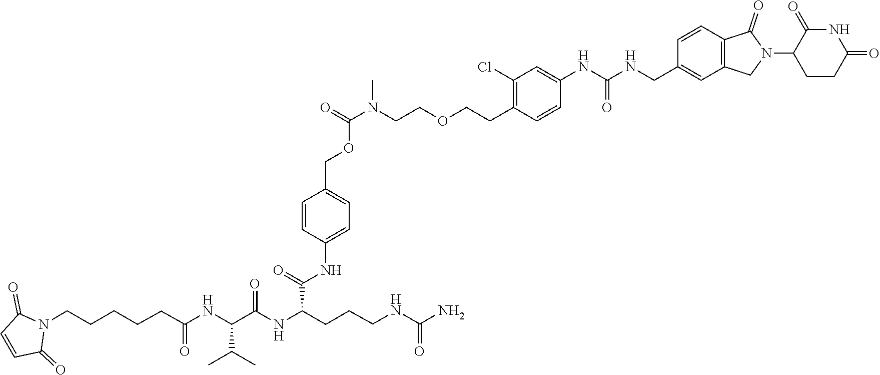 [SMILES: CC(C)[C@H](NC(=O)CCCCCN1C(=O)C=CC1=O)C(=O)N[C@@H](CCCNC(N)=O)C(=O)Nc1ccc(COC(=O)N(C)CCOCCc2ccc(NC(=O)NCc3ccc4c(c3)CN(C3CCC(=O)NC3=O)C4=O)cc2Cl)cc1]

### Example 4: Synthesis of Compound (Ic)

To a stirred mixture of 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 200 mg, 0.38 mmol, 1.00 equiv) and lutidine (81 mg, 0.76 mmol, 2.00 equiv) in DMF (10 mL) were added HOBT (26 mg, 0.19 mmol, 0.50 equiv) and [4-[(2S)-5-(carbamoylamino)-2-[(2S)-2-[6-(2,5-dioxopyrrol-1-yl)hexanamido]-3-methylbutanamido]pentanamido]phenyl]methyl 4-nitrophenyl carbonate (279 mg, 0.38 mmol, 1.00 equiv) in portions at room temperature. The reaction mixture was stirred for 12 hours at 40 degrees C. under nitrogen atmosphere. After the reaction was cooled down to room temperature, the reaction was quenched with water (30 mL). The resulting mixture was extracted with DCM (3×30 mL). The combined organic layers were washed with water (2×30 mL), brine (30 mL), dried over Na₂SO₄. After filtration, the filtrate was concentrated to dryness under vacuum. The residue was purified by reverse phase column (C18, mobile phase A: 0.1% FA in water, B: ACN). The collected fraction was concentrated to dryness under vacuum. The crude product (60 mg) was purified by Prep-HPLC with the following conditions (Column: Xselect CSH OBD Column 30×150 mm 5 um, n; Mobile Phase A:Water(0.1% FA), Mobile Phase B:ACN; Flow rate:60 mL/min; Gradient:33 B to 50 B in 7 min; 220 nm; RT1:5.27 min). The collected fraction was lyophilized to afford [4-[(2S)-5-(carbamoylamino)-2-[(2S)-2-[6-(2,5-dioxopyrrol-1-yl)hexanamido]-3-methylbutanamido]pentanamido]phenyl]methyl N-[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-N-methylcarbamate (23.8 mg, 5%) as a white solid. LCMS (ESI): 1126.11 (M+H)⁺. ¹H NMR (400 MHz, DMSO-d6) δ 10.99 (s, 1H), 10.00 (s, 1H), 8.88 (s, 1H), 8.12-8.08 (m, 1H), 7.85-7.81 (m, 2H), 7.70-7.67 (m, 2H), 7.60-7.58 (m, 1H), 7.51 (s, 1H), 7.47-7.44 (m, 1H), 7.28-7.25 (m, 2H), 7.18-7.12 (m, 2H), 7.00 (s, 2H), 6.90 (br s, 1H), 5.97-5.95 (m, 1H), 5.42 (s, 2H), 5.12-5.05 (m, 1H), 4.98 (s, 2H), 4.42-4.32 (m, 4H), 4.18-4.15 (m, 1H), 3.56-3.40 (m, 4H), 3.37-3.36 (m, 3H), 3.05-2.90 (m, 3H), 2.89-2.85 (m, 5H), 2.72-2.55 (m, 2H), 2.40-2.33 (m, 2H), 2.25-2.15 (m, 2H), 2.00-1.87 (m, 2H), 1.74-1.57 (m, 2H), 1.50-1.42 (m, 5H), 1.22-1.10 (m, 3H), 0.85-0.80 (m, 6H).

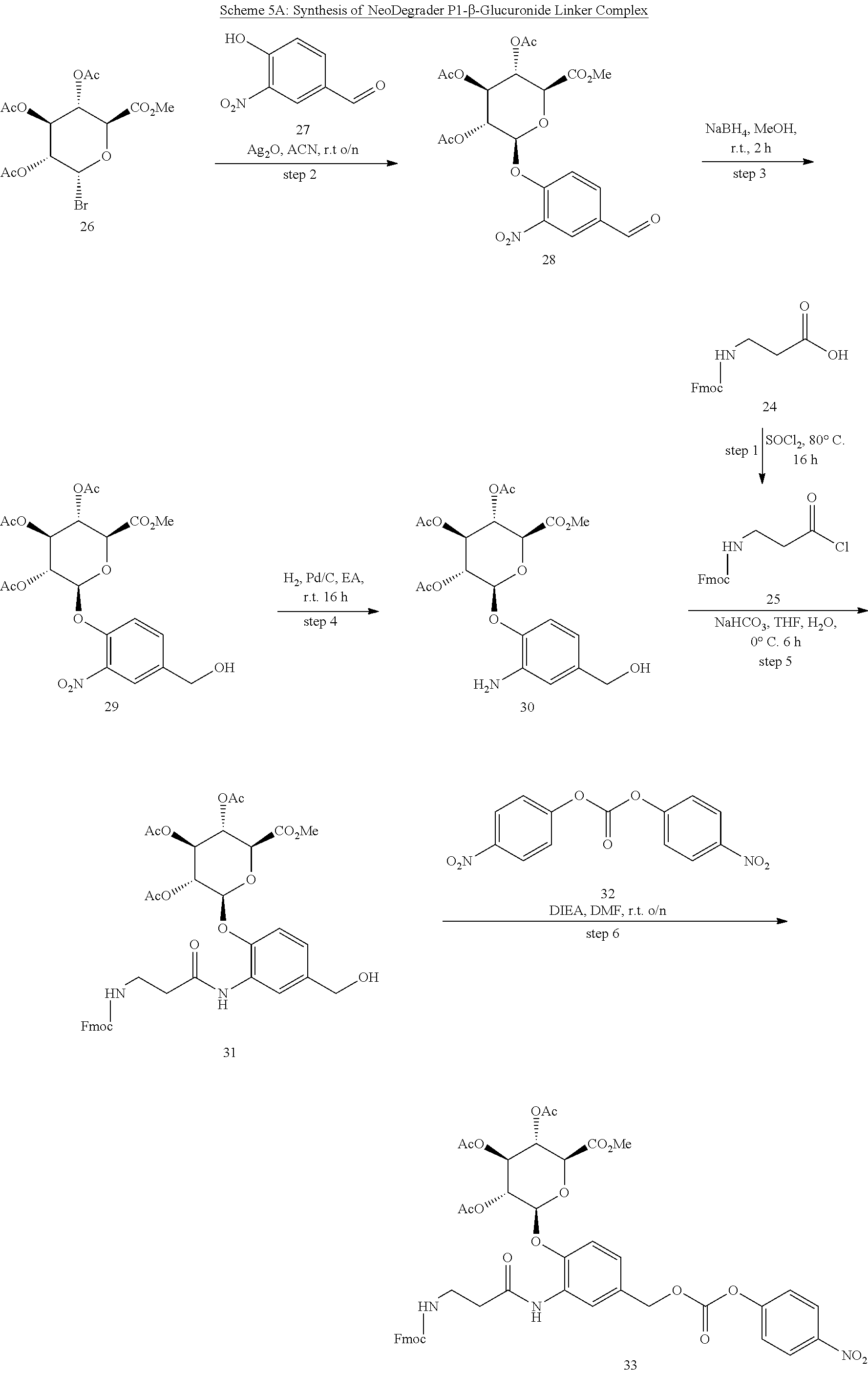

### Step 1. Synthesis of Compound 25

To a stirred mixture of 3-[[(9H-fluoren-9-ylmethoxy)carbonyl]amino]-propanoic acid (Compound 24, 5.00 g, 16.06 mmol, 1.00 equiv) in SOCl₂ (25 mL) at room temperature. The resulting mixture was stirred 16 h at 80° C. The desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 25, 7.5 g, crude) as a yellow oil. The crude product was used in the next step directly without further purification. ¹H-NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 28

To a stirred solution of 4-formyl-2-nitrophenol (Compound 27, 4.21 g, 25.19 mmol, 1.00 equiv) and Ag₂O (7.00 g, 30.20 mmol, 1.20 equiv) in ACN (100 mL, 190.24 mmol, 75.00 equiv) were added Compound 26 (10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 ml×3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 28, 10.5 g, 86%) as a white solid. ¹H-NMR analysis indicated it was the desired product. LCMS (ES, m/z):484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 29

To a stirred solution of methyl (2S,3 S,4S,5R,6S)-3,4,5-tris(acetyloxy)-6-(4-formyl-2-nitrophenoxy)oxane-2-carboxylate (Compound 28, 6.00 g, 12.41 mmol, 1.00 equiv) in MeOH (50 mL) were added NaBH₄ (0.47 g, 12.42 mmol, 1.00 equiv) in portions at RT under N₂ atmosphere. The resulting mixture was stirred for 2 h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate (Compound 29, 5.5 g, 91%) as a solid. LCMS (ES, m/z):486 [M+H]⁺.

### Step 4. Synthesis of Compound 30

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 29, 5.50 g, 11.33 mmol, 1.00 equiv) in EA (60 mL) were added Pd/C (1.10 g, 10%) in portions at room temperature. The resulting mixture was stirred for 16 h at room temperature under H2 atmosphere. LCMS indicated the reaction was completed. The resulting mixture was filtered, the filter cake was washed with DCM and MeOH, The filtrate was concentrated under vacuum to afford methyl (2S,3 S,4S,5R,6S)-3,4,5-tris(acetyloxy)-6-[2-amino-4-(hydroxymethyl)phenoxy]oxane-2-carboxylate (Compound 30, 4.0 g, 77%) as a solid. The crude product was used in the next step directly without further purification. LCMS (ES, m/z):456[M+H]⁺.

### Step 5. Synthesis of Compound 31

To a stirred solution of methyl (2S,3S,4S,5R,6S)-3,4,5-tris(acetyloxy)-6-[2-amino-4-(hydroxymethyl)phenoxy]oxane-2-carboxylate (Compound 30, 1.00 g, 2.19 mmol, 1.00 equiv) and NaHCO₃ (0.20 g, 2.40 mmol, 1.1 equiv) in THE (10 mL) was added Compound 25 (0.87 g, 2.62 mmol, 1.20 equiv) in portions at 0° C. under N₂ atmosphere. The resulting mixture was stirred for 6 h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100%) to afford methyl (2S,3S,4S,5R,6S)-3,4,5-tris(acetyloxy)-6-[2-(3-[[(9H-fluoren-9-ylmethoxy)carbonyl]amino]-propanamido)-4-(hydroxymethyl)phenoxy]oxane-2-carboxylate (Compound 31, 1.1 g, 66%) as a light yellow solid. LCMS (ES, m/z):749 [M+H]+.

### Step 6. Synthesis Compound 33

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 31, 1.50 g, 2.00 mmol, 1.00 equiv) and bis(4-nitrophenyl) carbonate (Compound 32, 0.68 g, 2.24 mmol, 1.12 equiv) in DMF (15 mL) was added DIEA (0.52 g, 4.01 mmol, 2.00 equiv) in portions at 0° C. under N₂ atmosphere. The resulting mixture was stirred overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.4 g, 48%) as a yellow solid. LCMS (ES, m/z):914 [M+H]⁺.

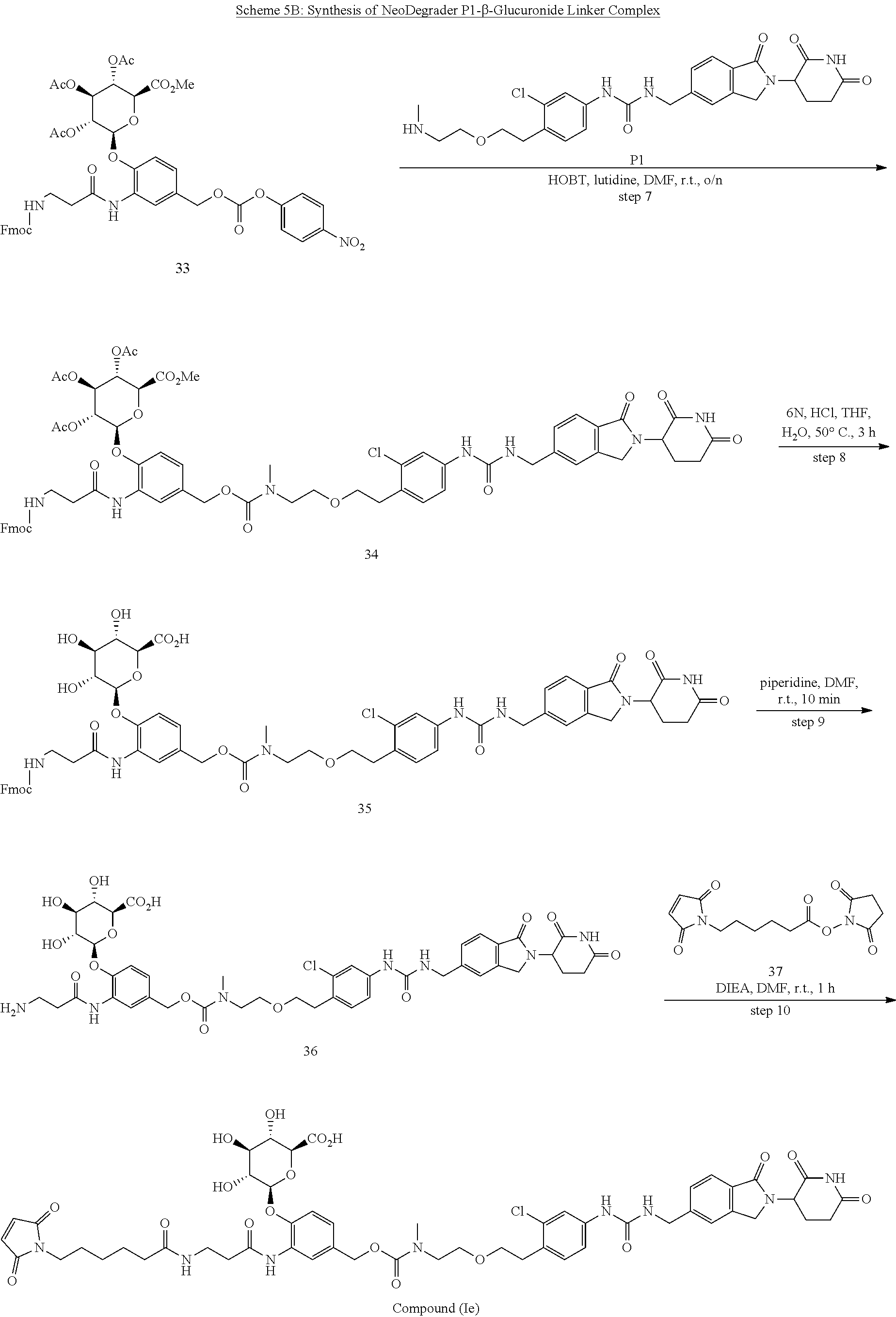

### Step 7. Synthesis Compound 34

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 33, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800 mg, 56%) as a solid. LCMS (ES, m/z):1302[M+H]⁺.

### Step 8. Synthesis Compound 35

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 34, 800.00 mg, 0.61 mmol, 1.00 equiv) in THE (80 mL) was added HCl (6N, 80 mL) in portions at room temperature under N₂ atmosphere. The resulting mixture was stirred for 3 h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 32%) as a white solid. LCMS (ES, m/z):1162[M+H]⁺.

### Step 9. Synthesis of Compound 36

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 35, 230 mg, 0.2 mmol, 1.00 equiv) in DMF (2 mL) was added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep HPLC with the following conditions (Column: XSelect CSH Prep C18 OBD Column, 19×250 mm, 5 um; Mobile Phase A: water (0.05% TFA), Mobile Phase B: ACN; Flow rate: 25 mL/min; Gradient:20 B to 40 B in 7 min; 220 nm; RT 1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]-ethoxy)ethyl](methyl)carbamoyl]oxy)methyl]phenoxy]-3,4,5-trihydroxyoxane-2-carboxylic acid (Compound 36.35 mg, 18%) as a white solid. LCMS (ES, m/z): 940[M+H]⁺.

### Step 10. Synthesis of Compound (Ie)

To a stirred solution of (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 36, 30 mg, 0.03 mmol, 1.00 equiv) in DMF (3 mL) were added DIEA (13 mg, 0.10 mmol, 3.00 equiv) and Compound 37 (30 mg, 0.10 mmol, 3.00 equiv) in portions at room temperature under nitrogen atmosphere. The resulting mixture was stirred for 1 h at room temperature under nitrogen atmosphere. LCMS indicated the reaction was completed. The resulting mixture was purified by Prep-HPLC with the following conditions (Column: Xselect CSH OBD Column 30×150 mm Sum, Mobile Phase A:water (0.1% FA), Mobile Phase B:ACN; Flow rate:60 mL/min; Gradient:21 B to 36 B in 10 min; 220 nm; RT 1:11.15 min). The collected fraction was lyophilzed to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl]-(methyl)carbamoyl]oxy)methyl]-2-[3-[6-(2,5-dioxopyrrol-1-yl)hexanamido]propanamido]phenoxy]-3,4,5-trihydroxyoxane-2-carboxylic acid (Compound (Ie), 10.5 mg, 28%) as a white a solid. LCMS (ES, m/z):1133[M+H]⁺. ¹H-NMR (300 MHz, DMSO-d6) δ 10.9 (s, 1H), 9.13 (s, 1H), 8.16 (s, 1H), 7.92-7.68 (m, 4H), 7.52 (s, 1H), 7.44 (d, J=3.0 Hz, 1H), 7.18-6.99 (m, 7H), 5.76 (s, 1H), 5.20-5.10 (m, 2H), 4.98 (br s, 2H), 4.76-4.74 (m, 1H), 4.42-4.33 (m, 4H), 3.65 (br s, 1H), 3.58-3.54 (m, 5H), 3.35 (d, J=6 Hz, 2H), 2.90-2.83 (m, 7H), 2.57-2.55 (m, 3H), 2.45-2.30 (m, 1H), 2.02-1.98 (m, 4H), 1.48-1.42 (m, 5H), 1.40-1.20 (m, 3H).

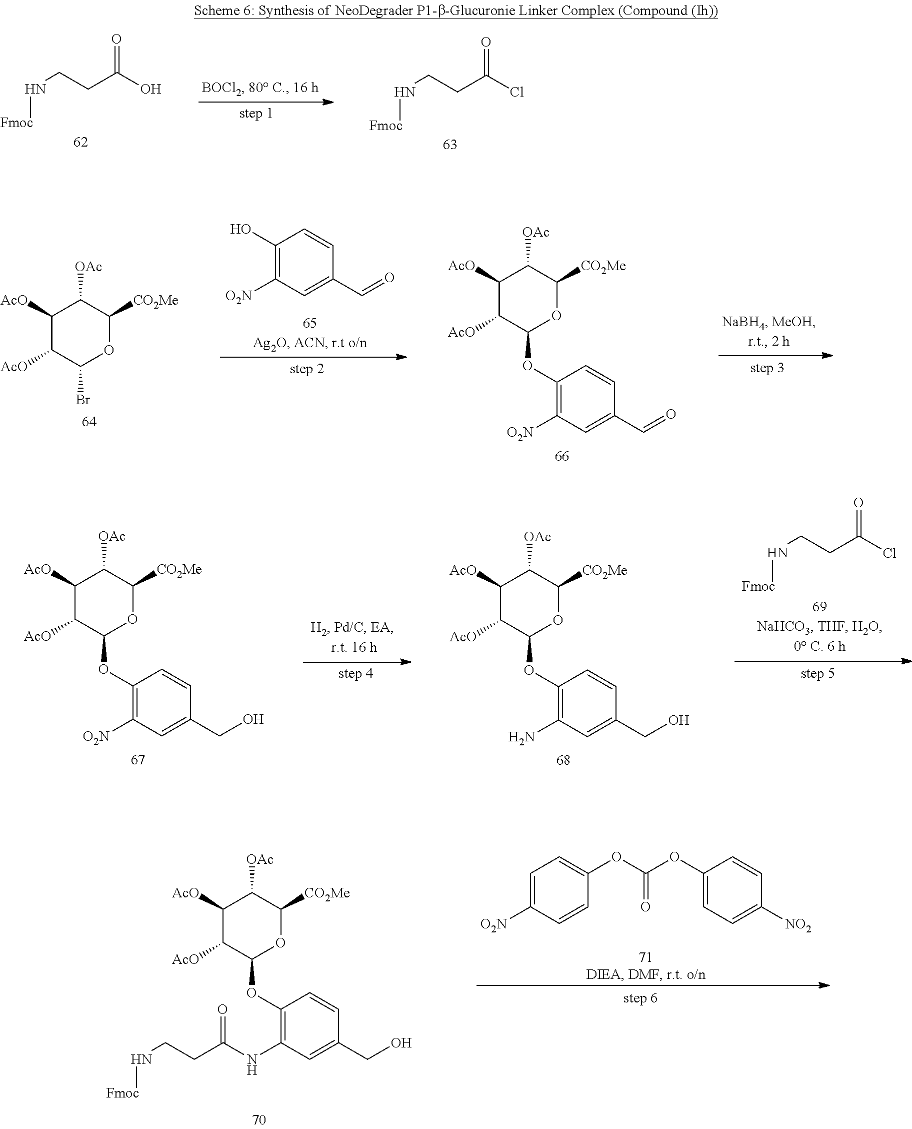

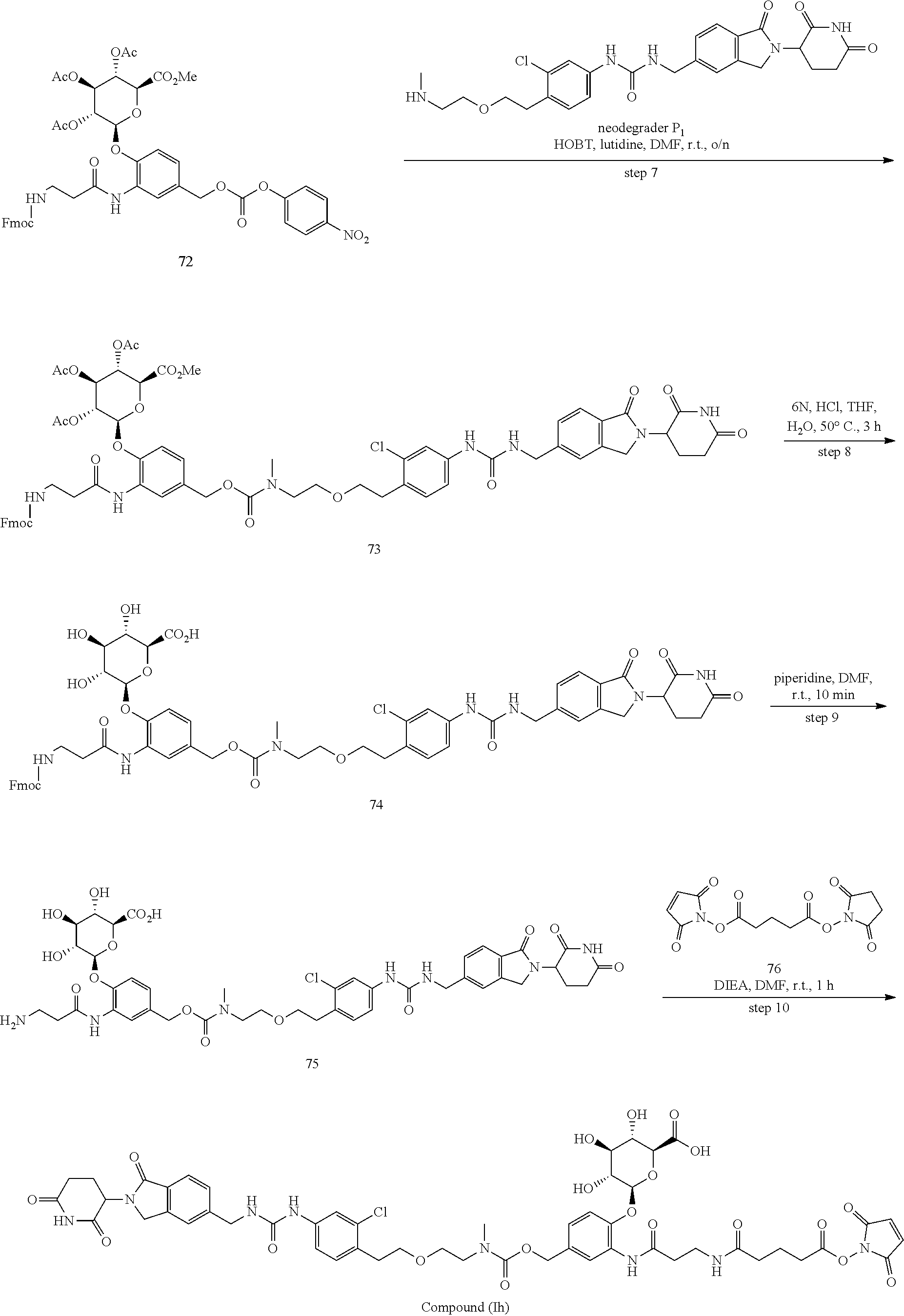

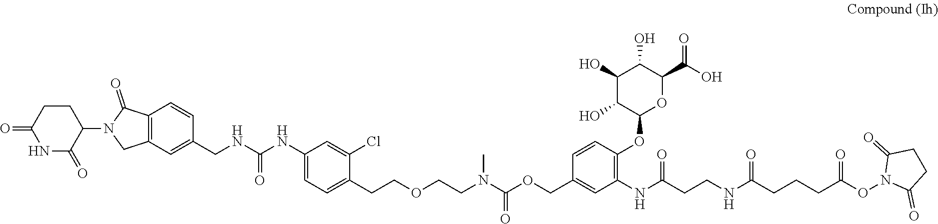 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(NC(=O)CCNC(=O)CCCC(=O)ON2C(=O)CCC2=O)c1]

### Example 6: Synthesis of Compound (Ih)

### Step 1. Synthesis of Compound 63

To a stirred mixture of 3-[[(9H-fluoren-9-ylmethoxy)carbonyl]amino]propanoic acid, (Compound 62, 5.00 g, 16.06 mmol, 1.00 equiv), SOCl₂ (25 mL) was added at room temperature. The resulting mixture was stirred 16 h at 80° C. Desired product could be detected by LCMS (derivative with MeOH MS=326). LCMS indicated the reaction was completed. The resulting mixture was concentrated under vacuum to afford 9H-fluoren-9-ylmethyl N-(3-chloro-3-oxopropyl)carbamate (Compound 63, 7.5 g, crude) as a yellow oil. The crude product was used directly in the next step without further purification. ¹H NMR analysis indicated it was the desired product (derivative with MeOH). ¹H-NMR (300 MHz, CDCl₃) δ 7.81-7.77 (m, 2H), 7.63-7.59 (m, 2H), 7.46-7.40 (m, 2H), 7.40-7.31 (m, 2H), 5.33 (s, 1H), 4.42 (d, J=3.0 Hz, 2H), 4.24 (t, J=6.0 Hz, 1H), 3.74-3.67 (m, 3H), 3.50 (d, J=3.0 Hz, 2H), 2.59 (t, J=6.0 Hz, 2H).

### Step 2. Synthesis of Compound 66

To a stirred solution of 4-formyl-2-nitrophenol (Compound 65, 4.21 g, 25.19 mmol, 1.00 equiv) and Ag₂O (7.00 g, 30.20 mmol, 1.20 equiv) in ACN (100 mL, 190.24 mmol, 75.00 equiv) were added methyl (2S,3S,4S,5R,6R)-3,4,5-tris(acetyloxy)-6-bromooxane-2-carboxylate (Compound 64, 10.00 g, 25.17 mmol, 1.00 equiv) in portions at room temperature under N₂ atmosphere. The resulting mixture was stirred for overnight at room temperature under N₂ atmosphere. LCMS indicated the reaction was completed. The resulting mixture was filtered, the filter cake was washed with DCM (50 mL×3). The filtrate was concentrated under reduced pressure. The residue was purified by silica gel column chromatography, eluted with PE/EA (PE:EA=1:2) to afford methyl (2S,3S,4S,5R,6S)-3,4,5-tris(acetyloxy)-6-(4-formyl-2-nitrophenoxy)oxane-2-carboxylate (Compound 66, 10.5 g, 86%) as a white solid. ¹H-NMR analysis indicated it was the desired product. LCMS (ES, m/z):484 [M+1]⁺. ¹H-NMR (300 MHz, CDCl₃) δ 10.00 (s, 1H), 8.34 (s, 1H), 8.13-8.09 (m, 1H), 7.52 (d, J=3.0 Hz, 1H), 5.47-5.29 (m, 4H), 4.37-4.35 (m, 1H), 3.75-3.73 (m, 3H), 2.17-2.06 (m, 9H).

### Step 3. Synthesis of Compound 67

To a stirred solution of methyl (2S,3 S,4S,5R,6S)-3,4,5-tris(acetyloxy)-6-(4-formyl-2-nitrophenoxy)oxane-2-carboxylate (Compound 66, 6.00 g, 12.41 mmol, 1.00 equiv) in MeOH (50 mL) was added NaBH₄ (0.47 g, 12.42 mmol, 1.00 equiv) in portions at room temperature under N₂ atmosphere. The resulting mixture was stirred for 2 h at room temperature under N₂ atmosphere. LCMS indicated the reaction was completed. The reaction was quenched with water at room temperature. The resulting was dried by Na₂SO₄. The resulting mixture was filtered, the filter cake was washed with DCM. The resulting mixture was concentrated under vacuum to afford methyl (2S,3S,4S,5R,6S)-3,4,5-tris(acetyloxy)-6-[4-(hydroxymethyl)-2-nitrophenoxy]oxane-2-carboxylate, (Compound 67, 5.5 g, 91%) as a solid. LCMS (ES, m/z):486 [M+H]+.

### Step 4. Synthesis of Compound 68

To a stirred mixture of methyl (2S,3S,4S,5R,6S)-3,4,5-tris(acetyloxy)-6-[4-(hydroxymethyl)-2-nitrophenoxy]oxane-2-carboxylate (Compound 67, 5.50 g, 11.33 mmol, 1.00 equiv) in EA (60 mL) were added Pd/C (1.10 g, 10%) in portions at room temperature. The resulting mixture was stirred for 16 h at room temperature under H2 atmosphere. LCMS indicated the reaction was completed. The resulting mixture was filtered, the filter cake was washed with DCM and MeOH, The filtrate was concentrated under vacuum to afford methyl (2S,3 S,4S,5R,6S)-3,4,5-tris(acetyloxy)-6-[2-amino-4-(hydroxymethyl)phenoxy]oxane-2-carboxylate (Compound 68, 4.0 g, 77%) as a solid. The crude product was used in the next step directly without further purification. LCMS (ES, m/z):456[M+H]+.

### Step 5. Synthesis of Compound 70

To a stirred solution of methyl (2S,3S,4S,5R,6S)-3,4,5-tris(acetyloxy)-6-[2-amino-4-(hydroxymethyl)phenoxy]oxane-2-carboxylate (Compound 68, 1.00 g, 2.19 mmol, 1.00 equiv) and NaHCO₃ (0.20 g, 2.40 mmol, 1.1 equiv) in THE (10 mL) were added 9H-fluoren-9-ylmethyl N-(3-chloro-3-oxopropyl)carbamate (Compound 69, 0.87 g, 2.62 mmol, 1.20 equiv) in portions at 0° C. under N₂ atmosphere. The resulting mixture was stirred for 6 h at 0° C. under N₂ atmosphere. LCMS indicated the reaction was completed. The reaction was quenched with water at room temperature. The resulting mixture was extracted with DCM. The combined organic layers were concentrated under reduced pressure. The residue was purified by silica gel column chromatography, eluted with PE/EA (EA=100%) to afford methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.1 g, 66%) as a light yellow solid. LCMS (ES, m/z):749 [M+H]⁺.

### Step 6. Synthesis of Compound 72

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-(hydroxymethyl)phenoxy]oxane-2-carboxylate (Compound 70, 1.50 g, 2.00 mmol, 1.00 equiv) and bis(4-nitrophenyl) carbonate (Compound 71, 0.68 g, 2.24 mmol, 1.12 equiv) in DMF (15 mL) were added DIEA (0.52 g, 4.01 mmol, 2.00 equiv) in portions at 0° C. under N₂ atmosphere. The resulting mixture was stirred for overnight at room temperature under nitrogen atmosphere. LCMS indicated the reaction was completed. The reaction mixture was purified by reverse flash chromatography with the following conditions: column, C18 silica gel; mobile phase, ACN in water (0.1% FA), 10% to 90% gradient in 40 min; detector, UV 254 nm. The collected fraction was concentrated to dryness in vacuum to afford methyl (2S,3S,4S,5R,6S)-3,4,5-tris(acetyloxy)-6-[2-(3-[[(9H-fluoren-9-ylmethoxy)carbonyl]-amino]propanamido)-4-[[(4-nitrophenoxycarbonyl)oxy]methyl]phenoxy]oxane-2-carboxylate (Compound 72, 1.4 g, 48%) as a yellow solid. LCMS (ES, m/z):914 [M+H]+.

### Step 7. Synthesis of Compound 73

To a stirred mixture of methyl (2S,3S,4S,5R,6S)-3,4,5-tris(acetyloxy)-6-[2-(3-[[(9H-fluoren-9-ylmethoxy)carbonyl]amino]propanamido)-4-[[(4-nitrophenoxycarbonyl)oxy]methyl]phenoxy]oxane-2-carboxylate (Compound 72, 1.00 g, 1.09 mmol, 1.00 equiv) and 1-(3-chloro-4-[2-[2-(methylamino)ethoxy]ethyl]phenyl)-3-[[2-(2,6-dioxopiperidin-3-yl)-1-oxo-3H-isoindol-5-yl]methyl]urea (neoDegrader P1, 0.58 g, 1.09 mmol, 1.00 equiv) in DMF (10 mL) were added HOBT (1.18 g, 8.72 mmol, 8.00 equiv) and 2,4-dimethylpyridine (1.07 g, 8.72 mmol, 8.00 equiv) in portions at room temperature under N₂ atmosphere. The resulting mixture was stirred for 16 h at room temperature under N₂ atmosphere. LCMS indicated the reaction was completed. The resulting mixture was used further purification. The residue was purified by reverse flash chromatography with the following conditions: column, C18 silica gel; mobile phase, ACN in water (0.1% FA), 10% to 80% gradient in 40 min; detector, UV 254 nm. The collected fraction was concentrated under vacuum to afford methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800 mg, 56%) as a solid. LCMS (ES, m/z):1302[M+H]+.

### Step 8. Synthesis of Compound 74

To a stirred mixture of methyl (2S,3S,4S,5R,6S)-3,4,5-tris(acetyloxy)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]oxane-2-carboxylate (Compound 73 (800.00 mg, 0.61 mmol, 1.00 equiv) in THE (80 mL) were added HCl (6N, 80 mL) in portions at room temperature under N₂ atmosphere. The resulting mixture was stirred for 3 h at degrees 50° C. under nitrogen atmosphere. LCMS indicated the reaction was completed. The resulting mixture was concentrated under vacuum. The residue was purified by reverse flash chromatography with the following conditions: column, C18 silica gel; mobile phase, ACN in water (0.1% FA), 0% to 80% gradient in 40 min; detector, UV 254 nm. The collected fraction was lyophilized to afford (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid (Compound 74, 230 mg, 32%) as a white solid. LCMS (ES, m/z):1162[M+H]⁺.

### Step 9. Synthesis of Compound 75

To a stirred solution of (2S,3S,4S,5R,6S)-6-[4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]-2-(3-[[(9H-fluoren-9-ylmethoxy)carbonyl]amino]propanamido)phenoxy]-3,4,5-trihydroxyoxane-2-carboxylic acid, 74 (230 mg, 0.2 mmol, 1.00 equiv) in DMF (2 mL) were added piperidine (0.4 mL) in portions at room temperature under nitrogen atmosphere. The resulting mixture was stirred for 10 min at room temperature under nitrogen atmosphere. LCMS indicated the reaction was completed. The resulting mixture was used directly further purification by Prep-HPLC with the following conditions (Column: XSelect CSH Prep C18 OBD Column, 19×250 mm,5 um; Mobile Phase A:water (0.05% TFA), Mobile Phase B:ACN; Flow rate: 25 mL/min; Gradient:20 B to 40 B in 7 min; 220 nm; RT1:5.78 min) to afford (2S,3S,4S,5R,6S)-6-[2-(3-aminopropanamido)-4-[([[2-(2-[2-chloro-4-[([[2-(2,6-dioxopiperidin-3-yl)-1-oxo-3H-isoindol-5-yl]methyl]carbamoyl)amino]phenyl]ethoxy)ethyl](methyl)carbamoyl]oxy)methyl]phenoxy]-3,4,5-trihydroxyoxane-2-carboxylic acid (Compound 75, 35 mg, 18%) as a white solid. LCMS (ES, m/z): 940[M+H]⁺.

### Step 10. Synthesis of Compound (Ih)

To a stirred solution of (2S,3S,4S,5R,6S)-6-[2-(3-aminopropanamido)-4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]phenoxy]-3,4,5-trihydroxyoxane-2-carboxylic acid (Compound 75, 110 mg, 0.12 mmol, 1.00 equiv) and bis(2,5-dioxopyrrolidin-1-yl) pentanedioate (Compound 76, 46 mg, 0.14 mmol, 1.2 equiv) in DMF (2.0 mL) was added DIEA (30 mg, 0.23 mmol, 2.0 equiv) in portions at room temperature under nitrogen atmosphere. The resulting mixture was stirred for 1 h at room temperature under nitrogen atmosphere. LCMS indicated the reaction was completed. The reaction mixture was purified by Prep-HPLC with the following conditions (Column: Kinetex EVO prep C18, 30*150, Sum; Mobile Phase A: Water(0.05% TFA), Mobile Phase B: ACN; Flow rate: 60 mL/min; Gradient: 21% B to 41% B in 7 min, 41% B; Wave Length: 254 nm; RT1(min): 5.8. The collected fraction was lyophilized to afford (2S,3 S,4S,5R,6S)-6-{4-[({[2-(2-{2-chloro-4-[({[2-(2,6-dioxopiperidin-3-yl)-1-oxo-3H-isoindol-5-yl]methyl}carbamoyl)amino]phenyl}ethoxy)ethyl](methyl)carbamoyl}oxy)methyl]-2-(3-{5-[(2,5-dioxopyrrolidin-1-yl)oxy]-5-oxopentanamido}propanamido)phenoxy}-3,4,5-trihydroxyoxane-2-carboxylic acid (Compound (Ih), 48 mg, 34% as a white solid. LCMS (ES, m/z): 1151 [M+H]*, 1173 [M+Na]⁺. ¹H-NMR (300 MHz, DMSO-d6): 12.80 (br s, 1H), 10.98 (s, 1H), 9.08 (s, 1H), 8.79 (s, 1H), 8.18 (s, 1H), 7.96 (s, 1H), 7.68-7.66 (m, 2H), 7.51 (s, 1H), 7.44 (d, J=8.1 Hz, 1H), 7.25-7.00 (m, 4H), 6.82-6.80 (m, 1H), 5.86 (s, 1H), 5.39-5.30 (m, 2H), 5.14-5.07 (m, 1H), 4.97 (s, 2H), 4.84 (d, J=7.2 Hz, 1H), 4.47-4.27 (m, 4H), 3.90 (d, J=9.6 Hz, 1H), 3.56-3.48 (m, 4H), 3.45-3.36 (m, 6H), 2.95-2.80 (m, 8H), 2.75-2.65 (m, 3H), 2.62-2.55 (m, 2H), 2.49-2.35 (m, 1H), 2.21-2.16 (m, 2H), 2.01-1.95 (m, 1H), 1.85-1.80 (m, 2H).

### Example 7: General Procedure for Preparation and Characterization of NeoDegrader Conjugates

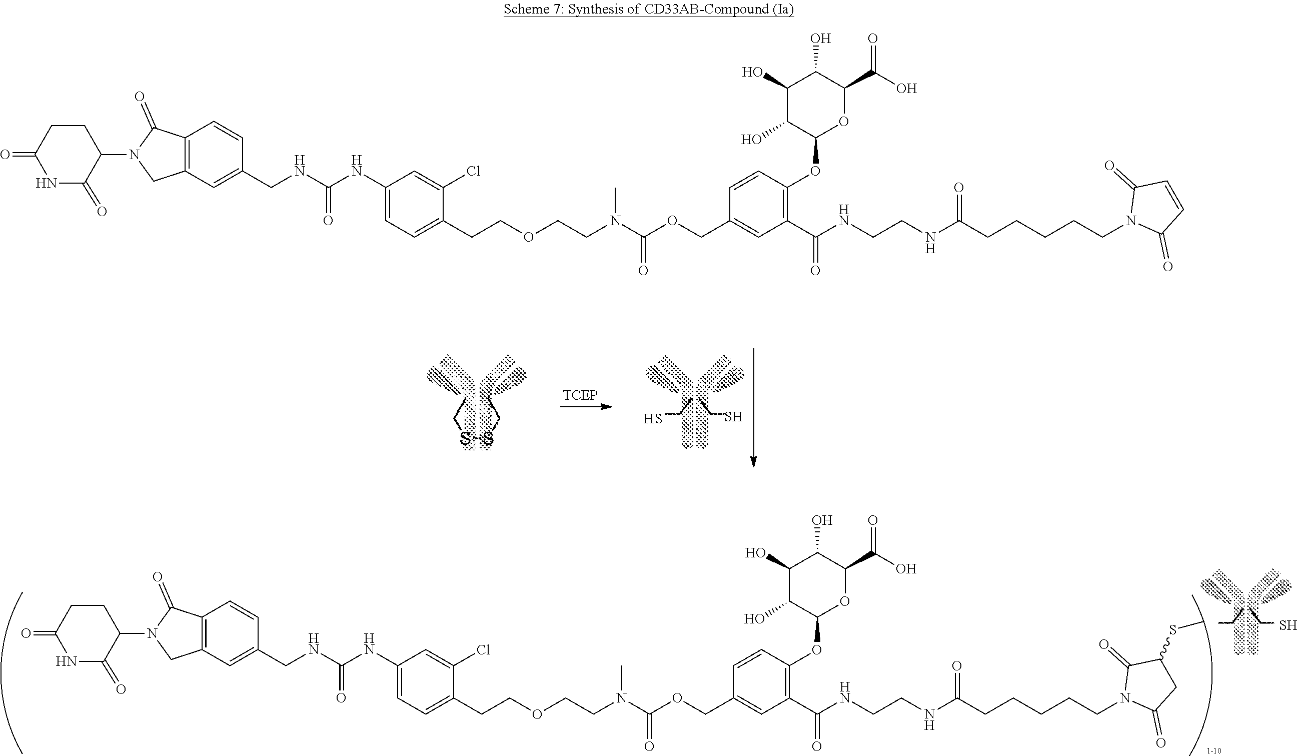

### Synthesis of CD33AB-Compound (Ia)

2.25 molar equivalents of TCEP were added to a 7.8 mg/mL solution of CD33AB in 50 mM EPPS, 5 mM EDTA pH 7.0 and the mixture was incubated for 2 hours at 37° C. After cooling the partially reduced antibody to ambient temperature, 8 molar equivalents of Compound (Ia) were added as a stock in solution in DMA such that the final concentration of antibody was 7.0 mg/mL and the final concentration of DMA was 10% (v/v). The reaction was incubated at ambient temperature for 1 hour. The resulting conjugate was purified into 20 mM sodium succinate, 8% sucrose, 0.01% Tween-20 pH 5.5 formulation buffer by gel filtration using Zeba 40K desalting columns followed by dialysis using Slide-a-Lyzer cassettes (10 K MWCO). The purified AnDC was found to have 100% monomer by SEC, an average drug loading of 3.1 drugs/antibody by reducing RPLC-MS, and <1.5% unconjugated Compound (Ia) by RPLC.

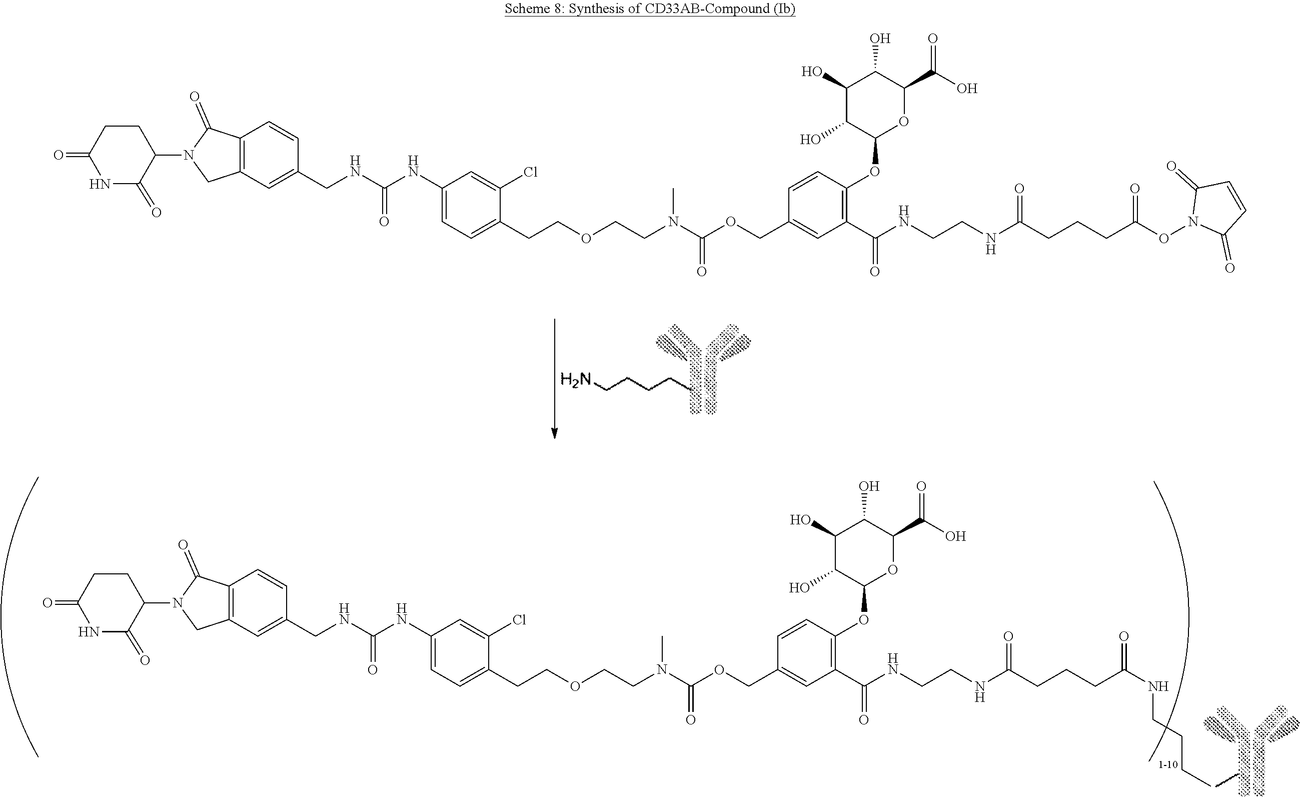

### Synthesis of CD33AB-Compound (Ib)

3.8 molar equivalents of Compound (Ib) were added as a stock solution in DMA to a solution of CD33AB in 50 mM EPPS pH 8.0 buffer such that the final concentration of antibody was 6.4 mg/mL and the final concentration of DMA was 10% (v/v). The reaction was incubated at ambient temperature for 3 hours. The resulting conjugate was purified into 20 mM sodium succinate, 8% sucrose, 0.01% Tween-20 pH 5.5 formulation buffer by gel filtration using Zeba 40K desalting columns followed by dialysis using Slide-a-Lyzer cassettes (10 K MWCO). The purified AnDC was found to have 100% monomer by SEC, an average drug loading of 3.1 drugs/antibody by intact RPLC-MS, and <1.5% unconjugated Compound (Ib) by RPLC.

Concentration and monomer were determined by size exclusion chromatography using a 7.8×300 mM TSKGel 3000SWXL column with 5 μm particles (Tosoh Bioscience), eluting isocratically with 400 mM sodium perchlorate, 50 mM sodium phosphate, 5% (v/v) isopropanol mobile phase running at 0.5 mg/mL for 30 min. NeoDegrader conjugates were quantitated from antibody standard curves, detecting at 214 nm.

Drug to antibody ratio (DAR) was determined by hydrophobic interaction chromatography using a 4.6×35 mm TSKgel Butyl-NPR column with 2.5 μm particles. Mobile phase A was 1.5 M ammonium sulfate, 25 mM sodium phosphate pH 7.0. Mobile phase B was 25 mM sodium phosphate pH 7.0, 25% (v/v) isopropanol. Analytes were eluted with a linear gradient of 0-100% B in 12 min. at a flow rate of 0.6 mL/min. Detection was at 214 nm.

Free linker-payload was determined by mixed-mode chromatography using a 4.6×250 mm HISEP column with 2.5 μm particles (Supelco). Mobile phase A was 100 mM ammonium acetate. Mobile phase B was 100% acetonitrile. Analytes were eluted with a gradient of 25-40% B in 25 min., then 40-100% B in 2 min at a flow rate of 0.7 mL/min. Column temperature was 35° C. Free linker-payload was quantitated using an external standard curve, detecting at 254 nm.

Additional neoDegrader conjugates can be prepared using the procedures described above, substituting the appropriate linker-neoDegrader with lysine or cysteine reactive conjugation handles.

### Example 8: Treatment of Acute Myeloid Leukemia (AML) with Anti-CD33 Antibody-neoDegrader Conjugate

CD33AB-neoDegrader compounds were tested in athymic nude mice (Crl:NU(NCr)-Foxn1^{nu}, Charles River). 1×10⁷ MV411 human acute monocytic leukemia cells (ATCC® CRL-5991™) in 50% Matrigel were injected subcutaneously in the flank of the mice (0.1 mL/mouse). The mice were dosed with anti-CD33 antibody-neoDegrader conjugates, non-targeting neoDegrader conjugates, and vehicle control once tumors reached an average size of 100-150 mm³.

The stock solutions of CD33AB-Compound (Ia), CD33AB-Compound (Ib) were diluted with vehicle to obtain 0.302 and 0.294 mg/mL dosing solutions, which provided, 3.02 and 2.94 mg/kg in a dosing volume of 10 mL/kg (0.2 mL per 20 g mouse), adjusted to the body weight of each animal. This dosing strategy ensured the delivery of the same amount of payloads to each testing group. Mylotarg was diluted in 0.9% sodium chloride solution to 0.01 mg/mL, which provided 3 mg/kg in a dosing volume of 10 mL/kg (0.2 mL per 20 g mouse). Venetoclax was formulated in solvent composed of 60% PG, 30% PEG400, 10% ethanol via ultrasonication to obtain a dosing suspension of 5 mg/mL, which delivered 50 mg/kg when administered in a volume of 10 mL/kg. CC-90009 was centrifuged to collect the powder at the bottom; then N-methyl-2-pyrrolidinone (NMP), PEG400 and saline were added and mixed well one by one to obtain a 0.5 mg/mL dosing solution in 5% NMP, 45% PEG400 and 50% saline, which delivered 5 mg/kg when administered in a volume of 10 mL/kg.

Mice were divided into 6 treatment groups (N=9/group), as follows: 1) vehicle; 2) CD33AB-Compound (Ia) (3.02 mg/kg, iv, qd×1); 3) CD33AB-Compound (Ib) (2.94 mg/kg, iv, qd×1); 4) Mylotarg (0.1 mg/kg, iv, qd×1); 5) Venetoclax (50 mg/kg, po, qd×21); 6)CC-90009 (5 mg/kg, ip, bid×10). Test articles for groups 1-4 were administered intravenously (i.v.) as a single dose (qd×1) in volumes adjusted for body weight (0.200 mL/20 g mouse). Venetoclax was administered orally (po) while CC-90009 was administered intraperitoneally (ip) in a dosing volume of 10 mL/kg (0.2 mL per 20 g mouse) scaled to the BW of each animal.

Tumors were measured using calipers twice per week, and each animal was euthanized when its tumor reached the endpoint volume (2,000 mm³) or on the last day (Day 45) of the study, whichever came first. The MTV(n) was defined as the median tumor volume on the last day of the study in the number of animals remaining (n) whose tumors had not attained the endpoint volume.

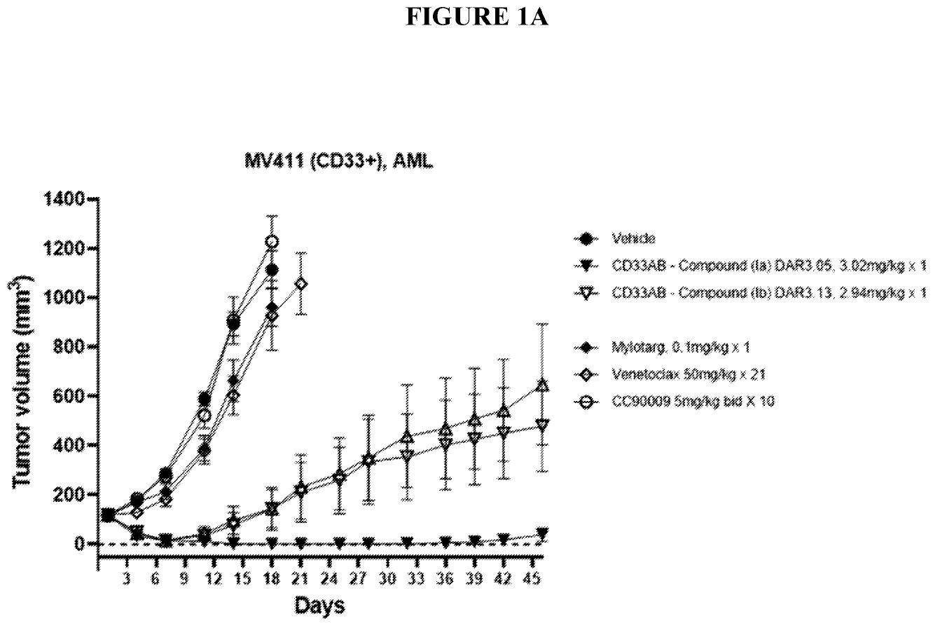

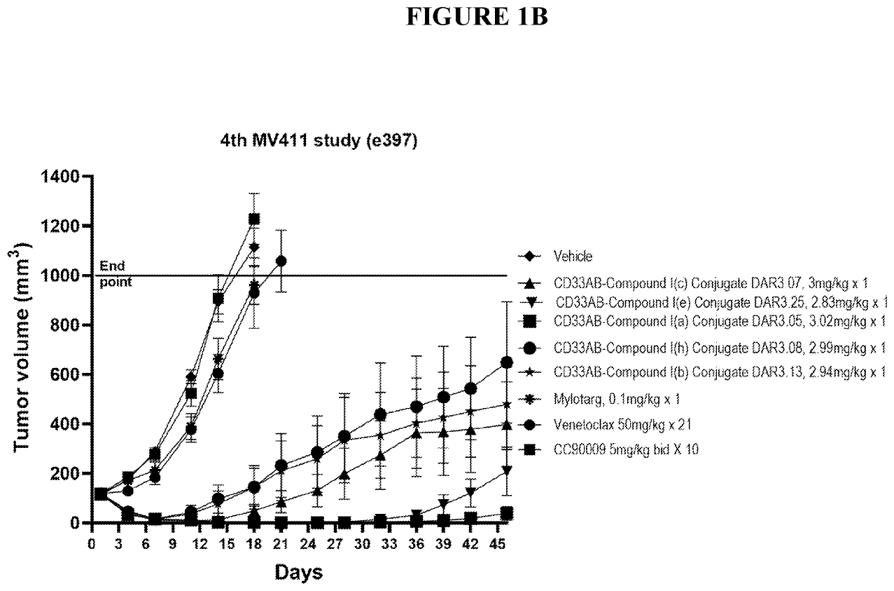

As shown in FIG. 1A, both of the neoDegrader conjugates provided slower tumor growth over time compared to the vehicle.

To further confirm activity, and evaluate alternative conjugation and linker-release modalities, a panel of CD33AB-based conjugates were tested and compared to Mylotarg—a clinically approved CD33-targeting ADC, and CC-90009—a small-molecule GSPT1 degrader in clinical trials at the clinical dosing levels, respectively. As shown in FIG. 1B, and consistent with the observations in vitro, in vivo treatment of a CD33-positive AML model tumor (MV4-11) with CD33AB-based conjugates, releasing neoDegrader P1 resulted in tumor regressions, with the most robust effects seen with a conjugate containing a beta-glucoronide release trigger and cysteine conjugation. Comparison of two variants of the beta-glu linker (Compound (Ie) vs Compound (Ia)) showed that the Compound (Ia) conjugate exhibited a response with longer durability as compared to Compound (Ie).

### Example 9: Treatment of Human Leukemia Models with Anti-CD33 Antibody-neoDegrader Conjugate

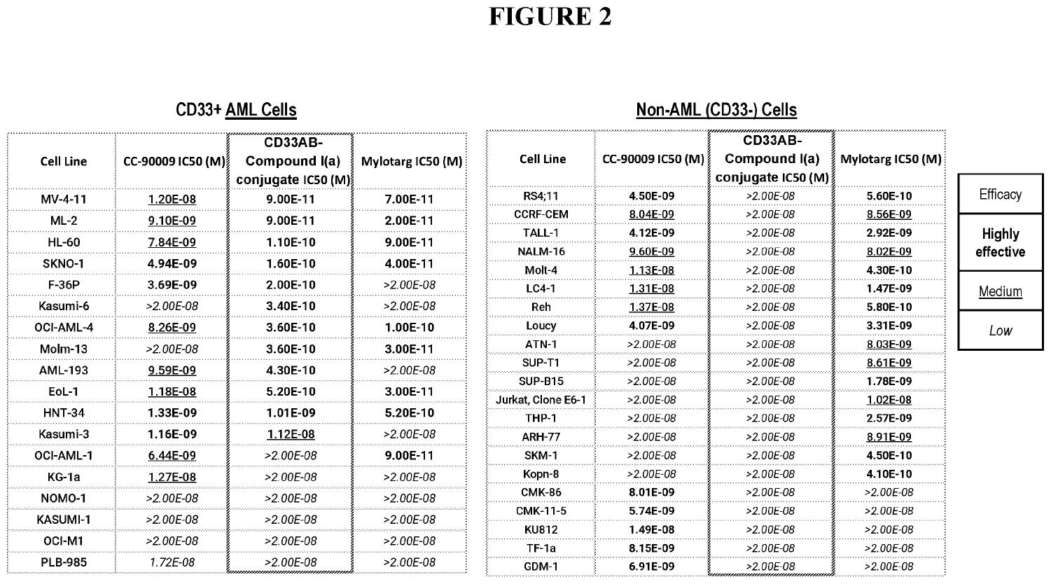

To confirm activity across a spectrum of CD33-positive and CD33-negative models, the efficacy of the CD33AB-Compound (Ia) conjugate to induce tumor cell killing in an in vitro panel of human leukemia models (including CD33-positive AML and CD33-negative malignancies) was evaluated. The cytotoxicities of test articles (TA) were measured using a panel of CD33-positive acute myeloid leukemia cell lines and a panel of non-AML CD33-negative cells. The cells, at a predetermined concentration, were plated into 96 well plates, and, after overnight incubation at 37° C./5% CO₂, serial dilutions of each test article (TA) were added to the cells. Cells were incubated with test articles for 72 hours, and viability was detected with CellTiter-Glo® reagent (Promega). The luminescent values were normalized for each cell line, and the IC50s were calculated using Prizm software. Results are shown in FIG. 2. While the conjugate showed good activity in several CD33+ cells, advantagenously, the conjugate was inactive in CD33-negative cell models.

### Example 10: Cytotoxicity of Anti-CD33 Antibody-neoDegrader Conjugate

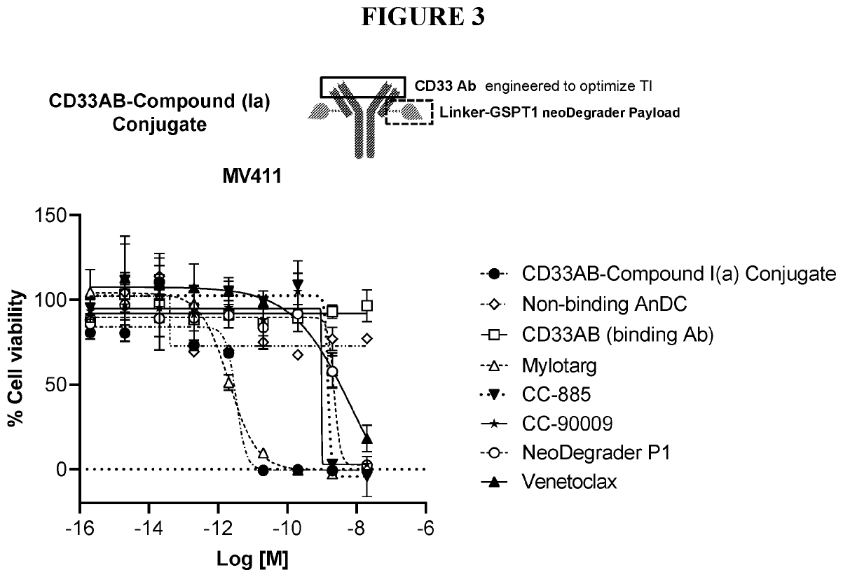

The cytotoxicity of the conjugate was measured using MV4-11 CD33-positive acute myeloid leukemia cells. The cells were plated in 96 well plates in assay media, and serial dilutions of CD33AB-Compound I(a) conjugate, Venetoclax, Mylotarg, CC-885, CC-90009, CD33AB (unconjugated antibody), or non-binding AnDC control, prepared in the assay media, were added to the cells. The cells were incubated for 72 hours, and then cell viability was detected with Cell Counting kit-8 (Dojindo) or CellTiter-Glo® reagent (Promega). As shown in FIG. 3, results indicated that the conjugate exhibited comparable overall in vitro efficacy as CC885 or Mylotarg CD33-positive AML cells—with some cases of superior efficacy.

### Example 11: Activity of Anti-CD33 Antibody-neoDegrader Conjugate in AML Patient Derived Cells

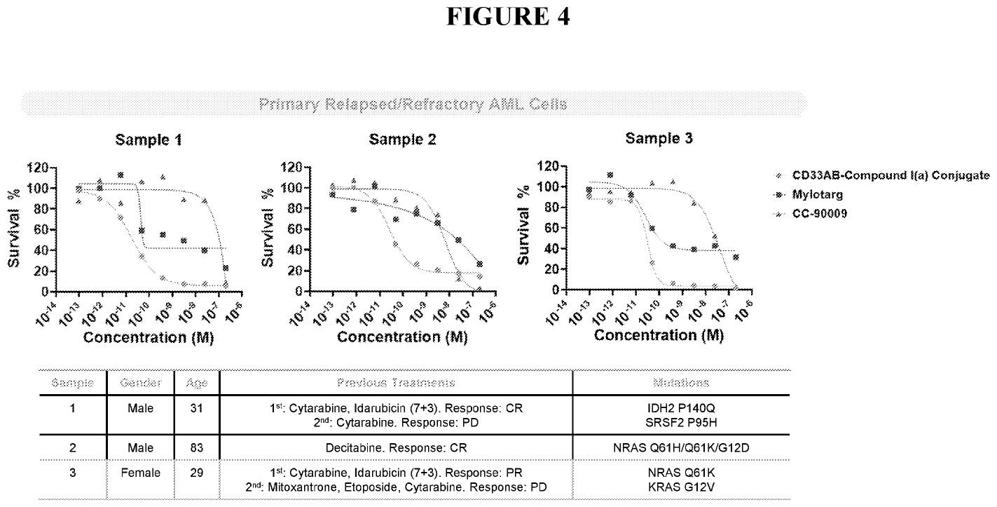

CD33AB-Compound (Ia) conjugate was evaluated in vitro in several AML patient primary blast cells. Frozen bone marrow samples from adult AML patients collected after their last treatment were defrosted and plated into 96 well plates containing serial dilutions of the conjugate, Mylotarg, or CC-90009. Plates were incubated for 48 hours at 37° C./5% CO₂, and then red blood cells in each well were lysed, and remaining cells were stained with blast marker antibody (best blast antibody for each donor was determined previously) together with Annexin V. Samples were analyzed by Flow Cytometry. Tumor cell survival was determined by absolute count of surviving tumor cells in each sample and normalized by the counts in the untreated wells. As shown in FIG. 4, the anti-CD33 antibody-neoDegrader conjugate exhibited superior activity, as measured by cytotoxicity, in patient-derived cells compared to either a standard-of-care treatment (Mylotarg) or an exploratory small-molecule GSPT1 degrader (CC-90009).

### Example 12: Activity of Anti-CD33 Antibody-neoDegrader Conjugate on Human Progenitor CFC Proliferation

As CD33 is expressed in normal bone-marrow progenitor cell populations, and normal cell myelosuppression is an observed adverse event (AE) for Mylotarg, the activities of the CD33AB-Compound (Ia) conugate, Mylotarg, and CC-90009 on normal erythroid, myeloid, and megakaryocyte progenitors were measured in colony forming assays. Normal human bone marrow light density cells were defrosted on the day of the experiment, washed, and plated into 24 well plates in XVivo 15 media supplemented with rhIL-3 (10 ng/mL), rhGMCSF (10 ng/mL), and rhSCF (50 ng/mL). CD33AB-Compound I(a) conjugate, Mylotarg, and CC-90009 were added to the wells, and cells were incubated for 72 hours. Following incubation, 400 μL of each cell suspension were added to the tubes containing methylcellulose-based medium supplemented with rhIL-3 (10 ng/mL), rh SCF (50 ng/mL), rhGM-CSF (10 ng/mL), and rhEpo (3 U/mL) for myeloid and erythroid progenitors. For megakaryocyte progenitors, cells were added to 35 mm dishes containing semi-solid, collagen-based matrix, supplemented with rhIL-3 (10 ng/mL), rhIL-6 (10 ng/mL) and rhTpo (50 ng/mL). After 14 days of incubation, colonies of myeloid and erythroid progenitors were assessed microscopically. Colonies were divided by size into several categories: CFU-E (Colony-forming unit-erythroid; this colony-forming cell generates small colonies containing less than 200 erythroblasts) and BFU-E-erythroid progenitors (Burst-forming unit-erythroid; this is a more primitive colony-forming cell, and it generates larger colonies containing more than 200 erythroblasts), CFU-GM-granulocyte-monocyte progenitors (Colony-forming unit—granulocyte, macrophage; this myeloid colony-forming cell is capable of producing colonies with 40 or more granulocyte-monocyte and/or macrophage cells), and CFU-GEMM-multipotential progenitors (Colony-forming unit—granulocyte, erythroid, macrophage, megakaryocyte; this primitive colony-forming cell is capable of producing colonies containing erythroid cells as well as 20 or more granulocytes, macrophages, and megakaryocytes).

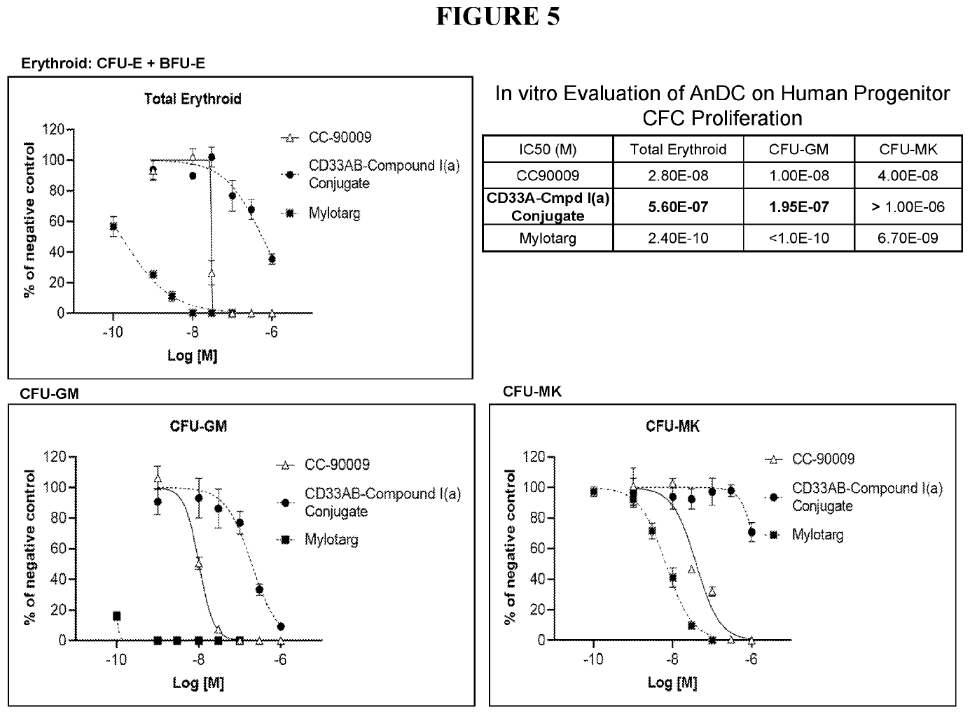

For megakaryocytes, following 14 days of incubation, cells were transferred to glass slides, fixed, and stained with CD41 antibody and alkaline phosphate detection system. Colonies were divided into three categories by size: CFU-Mk (3-20), CFU-Mk (21-49), CFU-Mk (>50). Results were normalized by the number of colonies in untreated samples and IC50s were calculated using Prizm software. The results are shown in FIG. 5.

### Example 13: Activity of Anti-CD33 Antibody-neoDegrader Conjugate in AML Tumors Compared to Current Standard of Care

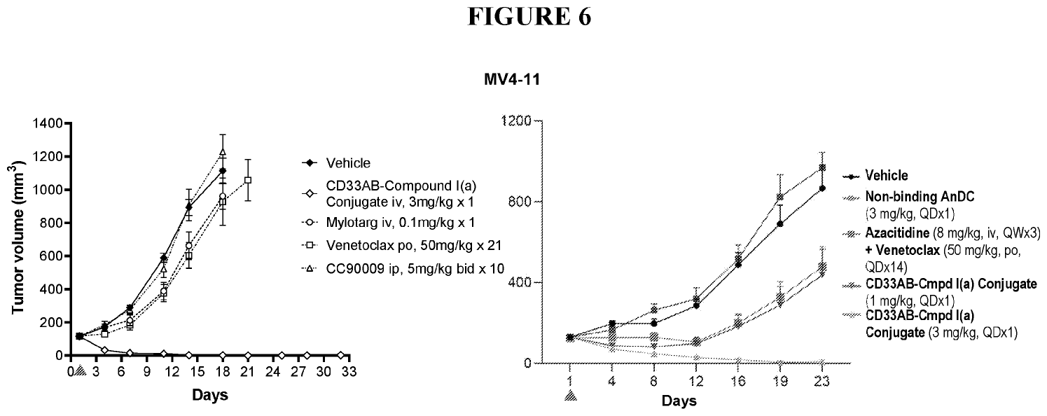

Subcutaneous tumor model MV4-11 human acute myelocytic leukemia cells (1×10⁶ cells in 0.1 mL) were subcutaneously inoculated into the right flank of female athymic nude mice. Mice were treated with TA either by intravenous injection into a lateral tail vein, intraperitoneal injection, oral gavage, or combinations thereof starting when tumors reached 150 mm³ in size. Tumor size and mouse body weight were measured twice per week. As shown in FIG. 6, the CD33AB-Compound (Ia) conjugate exhibited superior efficacy to best-available care options.

### Example 14: Activity of Anti-CD33 Antibody-neoDegrader Conjugate in Disseminated AML Models

As AML is a disease that is frequently disseminated throughout the patient, with lesions in multiple bone-marrow niches and circulating in the bloodstream, the activity of the CD33AB-Compound (Ia) conjugate in disseminated AML in vivo models was studied.

Disseminated model MV4-11 cells (3×10⁶ cells in 0.2 mL) were intravenously injected into the lateral tail vein of female NCG mice. Treatment was started thirteen days after tumor cell injection. Mice were checked daily for morbidity, mortality, and clinical observations. Body weight was measured twice per week. Imaging analysis of tumor development was conducted once per week under anesthesia by injection of 5 μL/g of D-luciferin 10 minutes prior to bioluminescent imaging.

Disseminated model OCI-AML2 human acute myelocytic leukemia cells (1×10⁷ cells in 0.2 mL) were intravenously injected into the lateral tail vein of female NOG mice. Nine days after tumor cell injection, treatment was started. Mice were checked daily for morbidity, mortality, and clinical observations. Body weight was measured twice per week. Imaging analysis of tumor development was conducted once per week under anesthesia by injection of 5 μL/g of D-luciferin 10 minutes prior to bioluminescent imaging.

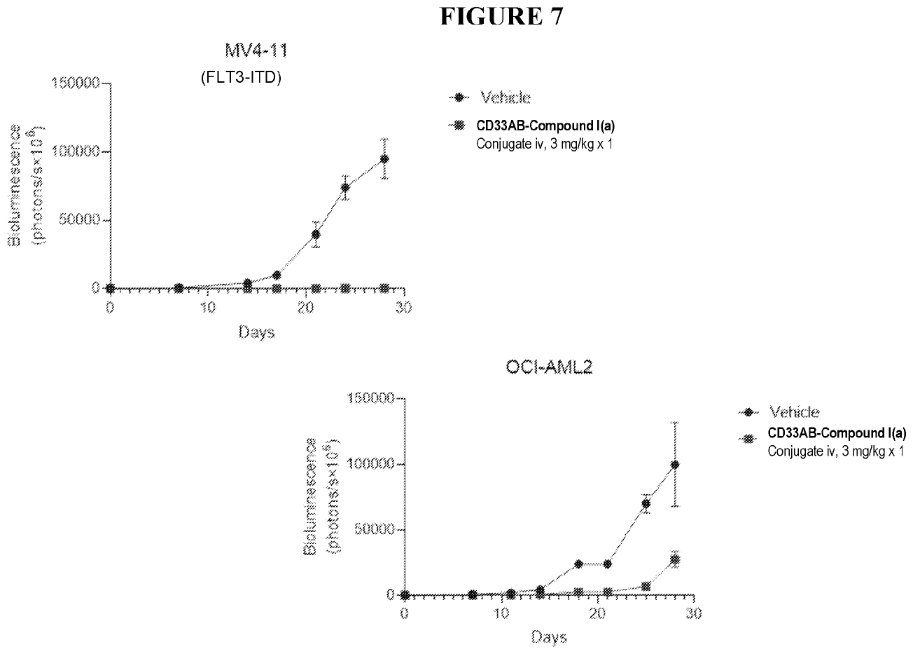

As shown in FIG. 7, a robust single-dose response in both models (MV4-11 and OCI-AML2) was seen, with durable regression of luciferin signal, a marker of the killing of the tumor cells.

### Example 15: Degradation of GSPT1 by CD33AB-Compound (Ia) Conjugate

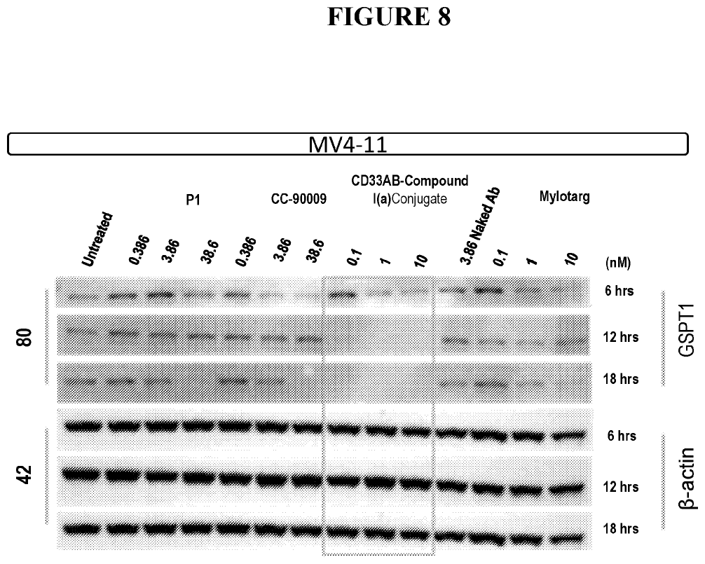

The CD33AB-Compound (Ia) conjugate's mechanism-of-action was confirmed by monitoring the degradation of GSPT1 by western blot. Whole cell lysates were prepared from MV4-11 CD33-positive AML cells treated by the CD33AB-Compound (Ia) conjugate, neoDegrader P1, CC-90009, or Mylotarg for 6, 12, and 18 hours, and then proteins were separated by electrophoresis and transferred onto polyvinylidene fluoride (PVDF) membrane. GSPT1 was probed by commercially available rabbit-anti-GSPT1 antibody (Abcam) followed by anti-rabbit HRP conjugated secondary antibody (Cell Signaling Technology) and detected by chemiluminescent substrate (ThermoFisher). PVDF membrane was then stripped and re-probed with beta-Actin-HRP conjugated antibody (Cell Signaling Technology). The results are shown in FIG. 8. These data support that conjugation of neoDegrader P1 to CD33AB enhances intracellular exposure to drive selective GSPT1 degradation. Similar dose-dependent decreases in GSPT1 levels were seen for the conjugate and the small molecules, neoDegrader P1 and CC-90009 in as short as 6 hours post-dosing. In contrast to the transient depletion seen with neoDegrader P1 and CC-90009—showing rebound in GSPT1 levels at the 12 hr timepoint, treatment with the conjugate showed durable depletion out to 18 hr post-dosing. These data support the potential for extended exposure of the active payload following dosing—consistent with the potential for a more profound duration of response and less frequent dosing.

### Example 16: Pharmacokinetic and Pharmacodynamic Efficacy of Anti-CD33 Antibody-neoDegrader Conjugate

Exposure and pharmacodynamic activity of the CD33AB-Compound (Ia) conjugate were evaluated relative to doses that were previously observed to establish tumor regression. Subcutaneous tumor model MV4-11 human acute myelocytic leukemia cells (1×10⁶ cells in 0.1 mL) were subcutaneously inoculated into the right flank of female athymic nude mice. Mice were treated with intravenous lateral tail vein injection of CD33AB-Compound (Ia) at 0.5 mg/kg, 1 mg/kg, and 3 mg/kg. Terminal cardiac puncture blood and tumors were collected from subcutaneous MV4-11 tumor model mice at pre-dose, 10 minutes, 30 minutes, 1 hour, 6 hour, 24 hour, 72 hour, and 120 hour timepoints following dosing. Blood was processed to EDTA plasma, and tumors were snap frozen in liquid nitrogen.

Pharmacokinetic analysis: Conjugate payload levels were quantified in the plasma samples by LC-MS/MS. The process comprised b-Glucuronidase digestion to release neoDegrader P1 payload from the conjugate followed by protein precipitation extraction to collect free and released payloads, and LC-MS/MS analysis using MRM acquisition. The method was qualified and met the acceptance criteria of non-GLP bioanalytical quantitation LC-MS/MS for small molecule analysis according to linearity, specificity, carryover, accuracy and precision. The relevant pharmacokinetic parameters were calculated using WinNonLin (V8.3).

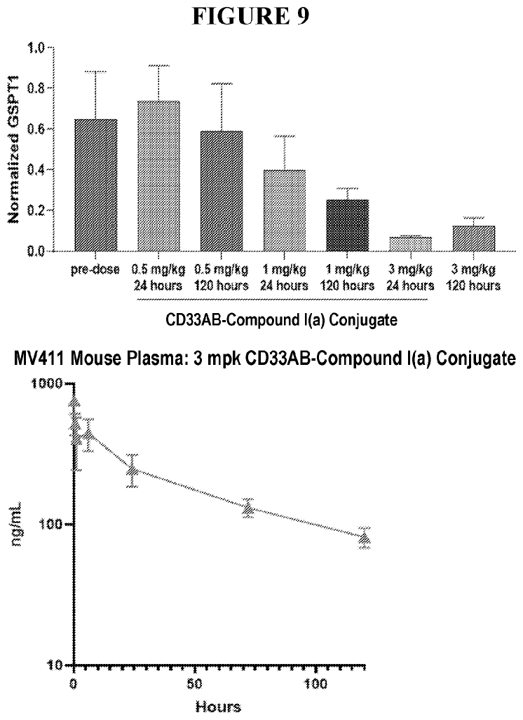

Pharmacodynamic analysis-tumor tissue was homogenized using RIPA lysis buffer with protease and phosphatase inhibitors, and western blotting was performed using a polyclonal rabbit anti-GSPT1 antibody (Abcam ab126090). Consistent with the durable depletion of GSPT1 seen in vitro, a single in vivo dose of the conjugate was sufficient to maintain reduced levels of GSPT1 for up to 120 hr (FIG. 9, top). In addition, conjugation of neoDegrader P1 to CD33AB resulted in a durable half-life of P1 of greater than 64 hr (FIG. 9, bottom), supporting durable exposure of the payload and the potential for less frequent dosing.

### Example 17: Activity of antiCD33 neoDegradader Conjugates Against Mylotarg-Insensitive Cell Lines

The in vitro cytotoxicity of test articles (TA) were measured using a panel of CD33 positive acute myeloid leukemia cell lines known to be Mylotarg insensitive (AML193 and Kasumi-6). The cells, at a predetermined concentration, were plated into 96 well plates, and, after overnight incubation at 37° C./5% C02, serial dilutions of each test article (TA) were added to the cells. Cells were incubated with test articles for 72 hours, and viability was detected with CellTiter-Glo® reagent (Promega). The luminescent values were normalized for each cell line, and the IC50s were calculated using Prizm software.

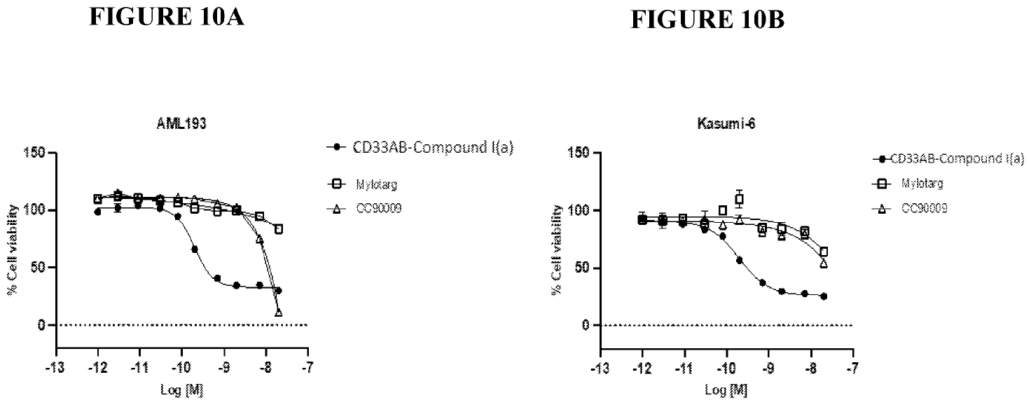

As shown in FIGS. 10A and 10B, the conjugate had good activity against both cell lines.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1-48. (canceled)

49. A conjugate of formula (I): 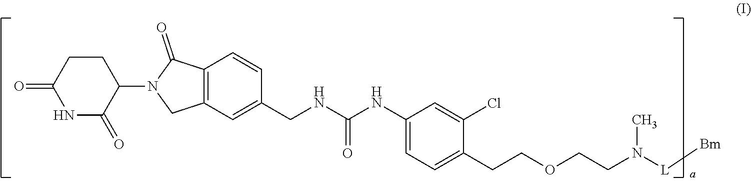
or a pharmaceutically acceptable salt thereof,
wherein:
a is 1 to 10;
L is 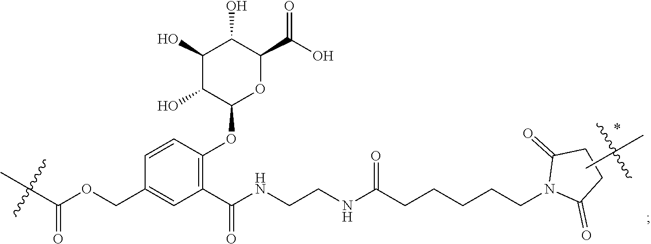 [symbol]
is the point of attachment to the nitrogen atom; [symbol]
is the point of attachment to Bm; and
Bm is an antibody comprising a heavy chain comprising the amino acid sequence as set forth in SEQ ID NO:9 and a light chain comprising the amino acid sequence as set forth in SEQ ID NO:10.

50. The conjugate of claim 49, wherein the conjugate is of formula (I).

51. The conjugate of claim 49, wherein the conjugate is the pharmaceutically acceptable salt of formula (I).

52. The conjugate of claim 49, or the pharmaceutically acceptable salt thereof, wherein a is 2 to 8.

53. The conjugate of claim 49, or the pharmaceutically acceptable salt thereof, wherein a is 3 to 5.

54. A conjugate of formula (I): 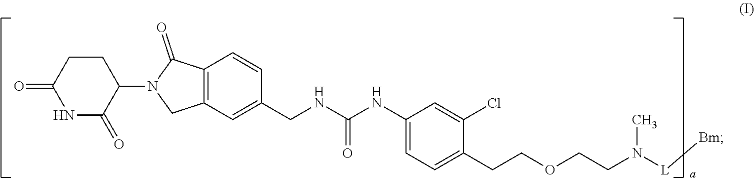
or a pharmaceutically acceptable salt thereof,
wherein:
a is 1 to 10;
L is 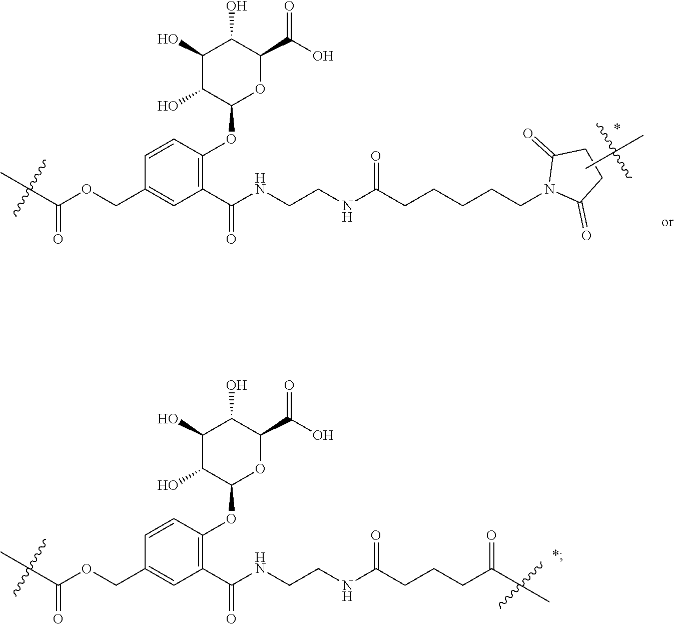 [symbol]
is the point of attachment to the nitrogen atom; [symbol]
is the point of attachment to Bm; and
Bm is an antibody or antigen binding portion thereof that is capable of specifically binding to CD33.

55. The conjugate of claim 54, or the pharmaceutically acceptable salt thereof, wherein a is 2 to 8, and L is

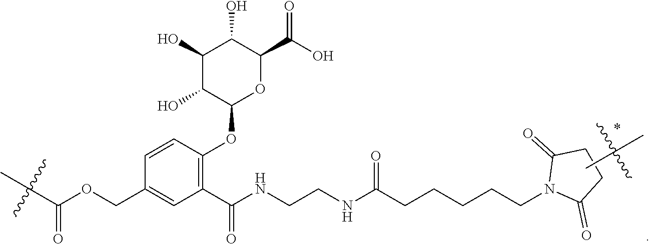

56. The conjugate of claim 55, or the pharmaceutically acceptable salt thereof, wherein a is 3 to 5.

57. The conjugate of claim 54, or the pharmaceutically acceptable salt thereof, wherein:
L is 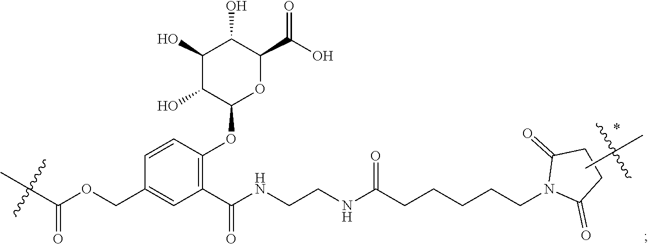
and
Bm is an antibody or an antigen binding portion thereof comprising a heavy chain variable region (VH) complementarity determining region (CDR) 1 (VH-CDR1), comprising the amino acid sequence as set forth in SEQ ID NO: 1, a VH-CDR2 comprising the amino acid sequence as set forth in SEQ ID NO: 2, a VH-CDR3 comprising the amino acid sequence as set forth in SEQ ID NO: 3, a light chain variable region (VL) CDR1 (VL-CDR1) comprising the amino acid sequence as set forth in SEQ ID NO: 5, a VL-CDR2 comprising the amino acid sequence as set forth in SEQ ID NO: 6, and a VL-CDR3 comprising the amino acid sequence as set forth in SEQ ID NO: 7.

58. The conjugate of claim 54, or the pharmaceutically acceptable salt thereof, wherein:
L is 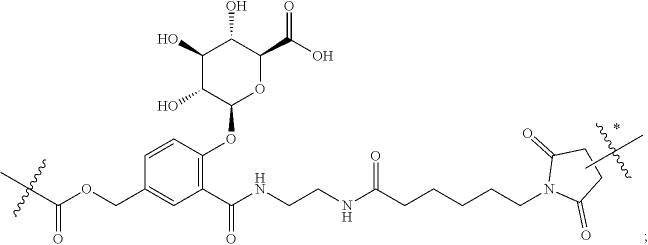
and
Bm is an antibody or an antigen binding portion thereof comprising a VH comprising the amino acid sequence as set forth in SEQ ID NO: 4 and a VL comprising the amino acid sequence as set forth in SEQ ID NO:8.

59. The conjugate of claim 54, or the pharmaceutically acceptable salt thereof, wherein Bm is an antibody comprising a heavy chain comprising the amino acid sequence as set forth in SEQ ID NO:9 and a light chain comprising the amino acid sequence as set forth in SEQ ID NO:10.

60. The conjugate of claim 54, wherein the conjugate is of formula (I).

61. The conjugate of claim 54, wherein the conjugate is the pharmaceutically acceptable salt of formula (I).

62. The conjugate of claim 54, or the pharmaceutically acceptable salt thereof, wherein L is covalently bound to Bm through a sulfur atom in Bm.

63. A pharmaceutical composition comprising the conjugate of claim 49, or the pharmaceutically acceptable salt thereof, and one or more pharmaceutically acceptable carriers.

64. A method of treating acute myeloid leukemia in a subject in need thereof, the method comprising administering to the subject a pharmaceutically acceptable amount of the conjugate of claim 49, or the pharmaceutically acceptable salt thereof.

65. A method of treating myelodysplastic syndrome in a subject in need thereof, the method comprising administering to the subject a pharmaceutically acceptable amount of the conjugate of claim 49, or the pharmaceutically acceptable salt thereof.

66. A method of treating cancer in a subject in need thereof, the method comprising administering to the subject a pharmaceutically acceptable amount of the conjugate of claim 49, or the pharmaceutically acceptable salt thereof.

67. The method of claim 66, wherein the cancer is breast cancer, gastric cancer, lymphoma, acute myeloid leukemia, multiple myeloma, head and neck cancer, squamous cell carcinoma, or hepatocellular carcinoma.

68. A compound of formula (II) or a pharmaceutically acceptable salt thereof:

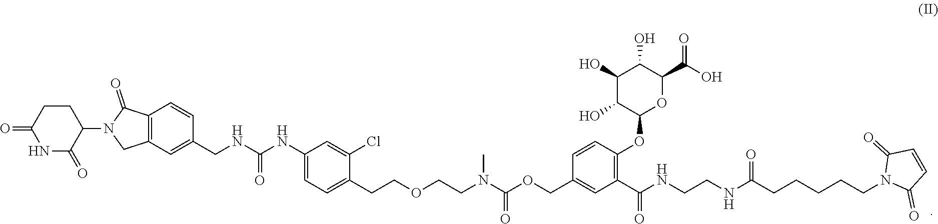 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]

69. A compound of formula (III) or a pharmaceutically acceptable salt thereof:

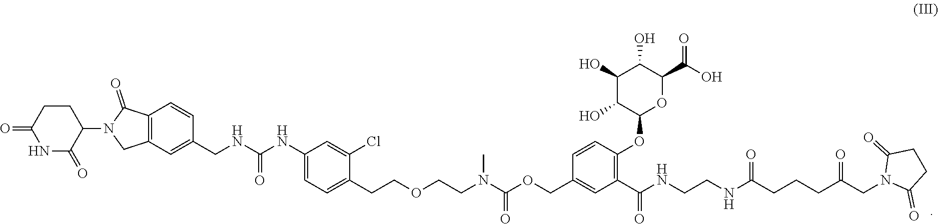 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCC(=O)CN2C(=O)CCC2=O)c1]

70. A compound selected from the group consisting of: 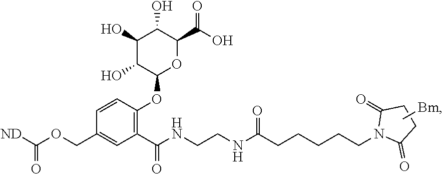
wherein ND is a neodegrader compound and Bm is an antibody or antigen binding portion thereof, 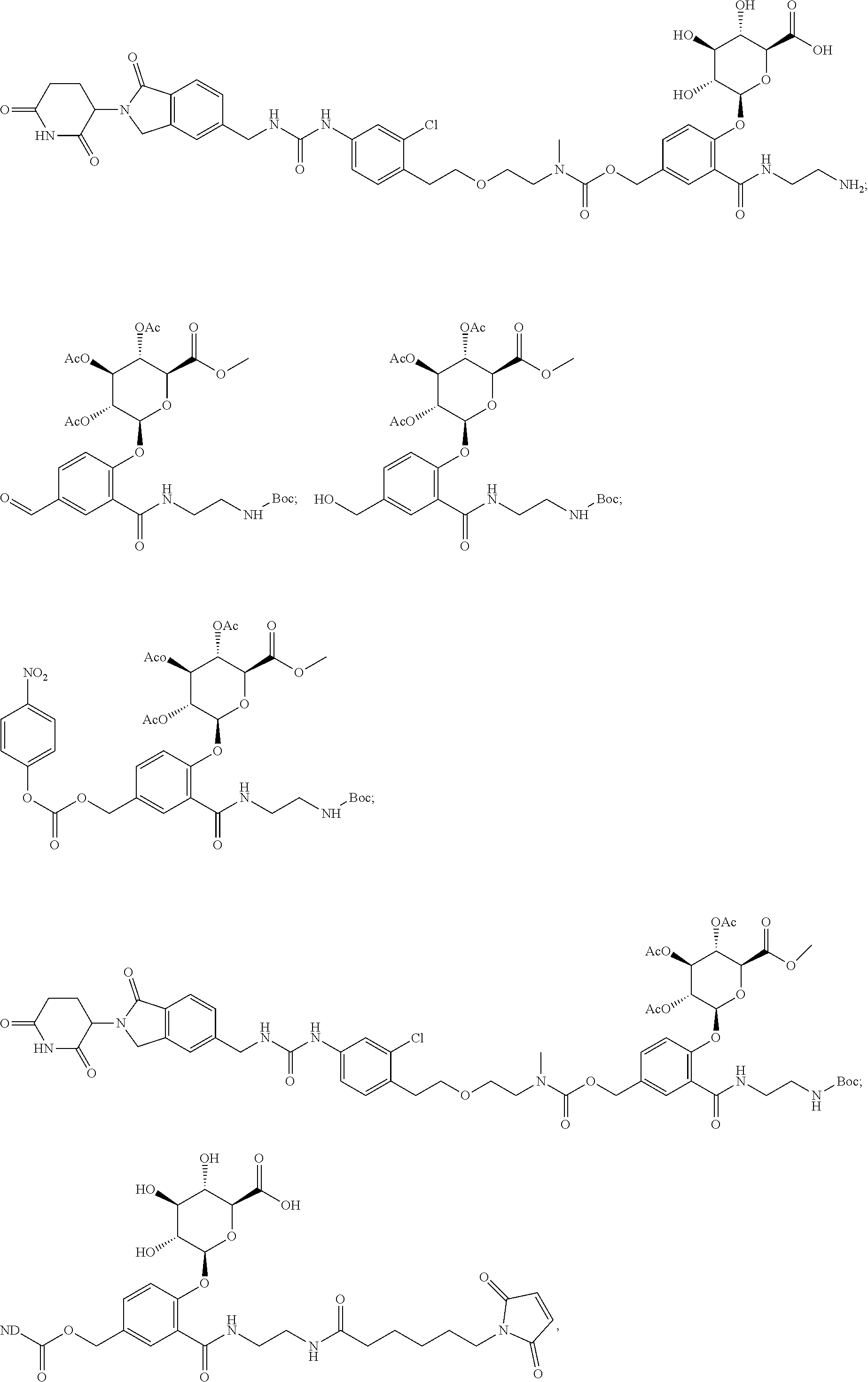
wherein ND is a neodegrader compound; 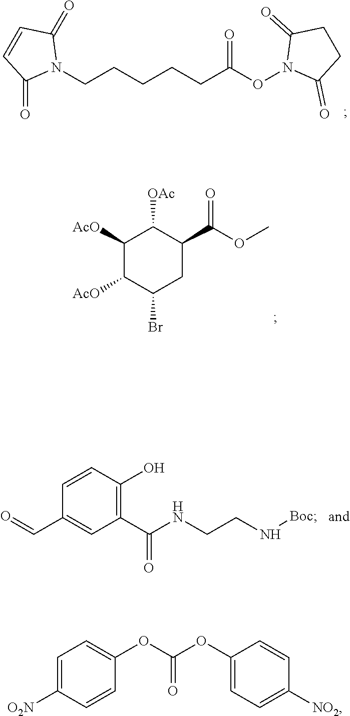 [SMILES: O=C(CCCCCN1C(=O)C=CC1=O)ON1C(=O)CCC1=O ; COC(=O)[C@H]1C[C@H](Br)[C@H](OC(C)=O)[C@@H](OC(C)=O)[C@@H]1OC(C)=O ; CC(C)(C)OC(=O)NCCNC(=O)c1cc(C=O)ccc1O ; "and" ; O=C(Oc1ccc([N+](=O)[O-])cc1)Oc1ccc([N+](=O)[O-])cc1]
or a pharmaceutically acceptable salt of any one of the foregoing.

71. An antibody comprising a heavy chain which comprises the amino acid sequence as set forth in SEQ ID NO:9 and a light chain which comprises the amino acid sequence as set forth in SEQ ID NO:10.

72. A process of preparing the conjugate of claim 54, or the pharmaceutically acceptable salt thereof, the process comprising reacting an antibody or antigen binding portion thereof with a compound of formula (I-1): 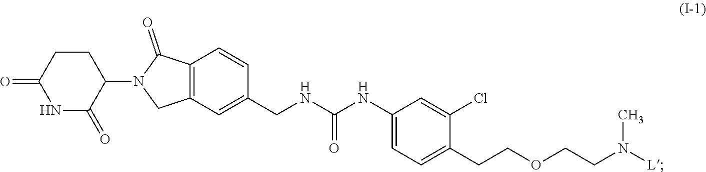
or a pharmaceutically acceptable salt thereof, wherein:
L′ is 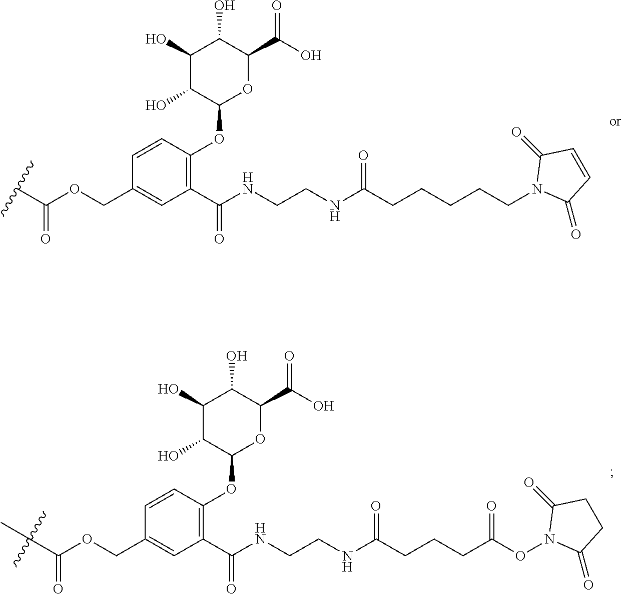 [SMILES: *C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1 ; *C(=O)OCc1ccc(O[C@@H]2O[C@H](C(=O)O)[C@@H](O)[C@H](O)[C@H]2O)c(C(=O)NCCNC(=O)CCCC(=O)ON2C(=O)CCC2=O)c1]
and [symbol]
is the point of attachment to the nitrogen atom.

73. A process selected from the group consisting of:
(a) a process for preparing compound 14 comprising reacting compound 12 with compound 13, thereby producing compound 14; wherein
compound 12 is: 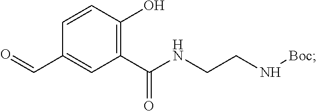 [SMILES: CC(C)(C)OC(=O)NCCNC(=O)c1cc(C=O)ccc1O]
compound 13 is: 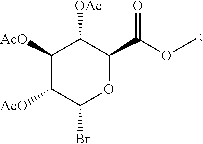 [SMILES: COC(=O)[C@H]1O[C@H](Br)[C@H](OC(C)=O)[C@@H](OC(C)=O)[C@@H]1OC(C)=O]
compound 14 is: 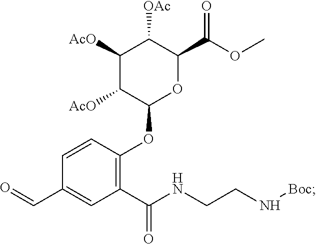 [SMILES: COC(=O)[C@H]1O[C@@H](Oc2ccc(C=O)cc2C(=O)NCCNC(=O)OC(C)(C)C)[C@H](OC(C)=O)[C@@H](OC(C)=O)[C@@H]1OC(C)=O]
(b) a process for preparing compound 15 comprising converting compound 14 to compound 15; wherein
compound 14 is:
and 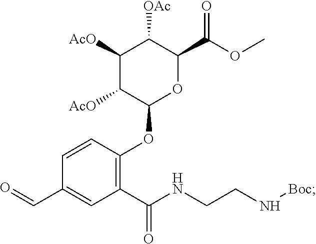 [SMILES: COC(=O)[C@H]1O[C@@H](Oc2ccc(C=O)cc2C(=O)NCCNC(=O)OC(C)(C)C)[C@H](OC(C)=O)[C@@H](OC(C)=O)[C@@H]1OC(C)=O]
compound 15 is: 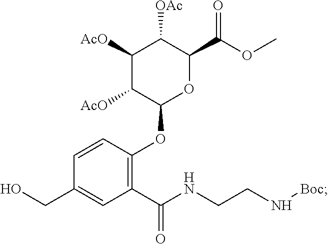 [SMILES: COC(=O)[C@H]1O[C@@H](Oc2ccc(CO)cc2C(=O)NCCNC(=O)OC(C)(C)C)[C@H](OC(C)=O)[C@@H](OC(C)=O)[C@@H]1OC(C)=O]
(c) a process for preparing compound 16 comprising reacting compound 15 with a compound having the formula 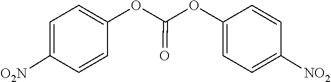 [SMILES: O=C(Oc1ccc([N+](=O)[O-])cc1)Oc1ccc([N+](=O)[O-])cc1]
to produce compound 16; wherein
compound 15 is: 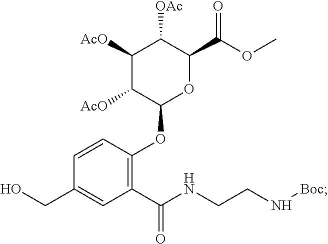 [SMILES: COC(=O)[C@H]1O[C@@H](Oc2ccc(CO)cc2C(=O)NCCNC(=O)OC(C)(C)C)[C@H](OC(C)=O)[C@@H](OC(C)=O)[C@@H]1OC(C)=O]
and
compound 16 is: 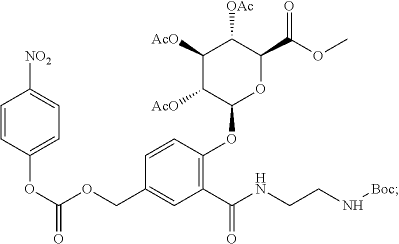 [SMILES: COC(=O)[C@H]1O[C@@H](Oc2ccc(COC(=O)Oc3ccc([N+](=O)[O-])cc3)cc2C(=O)NCCNC(=O)OC(C)(C)C)[C@H](OC(C)=O)[C@@H](OC(C)=O)[C@@H]1OC(C)=O]
(d) a process for preparing compound 17 comprising reacting compound 16 with a compound having the formula: 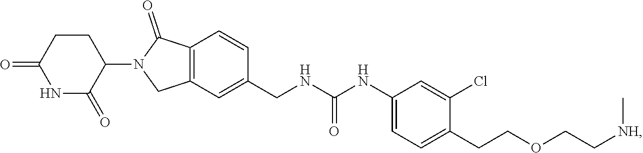 [SMILES: CNCCOCCc1ccc(NC(=O)NCc2ccc3c(c2)CN(C2CCC(=O)NC2=O)C3=O)cc1Cl]
thereby producing compound 17; wherein
compound 16 is: 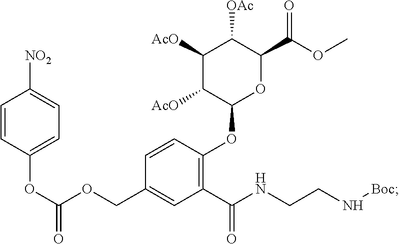 [SMILES: COC(=O)[C@H]1O[C@@H](Oc2ccc(COC(=O)Oc3ccc([N+](=O)[O-])cc3)cc2C(=O)NCCNC(=O)OC(C)(C)C)[C@H](OC(C)=O)[C@@H](OC(C)=O)[C@@H]1OC(C)=O]
and
compound 17 is: 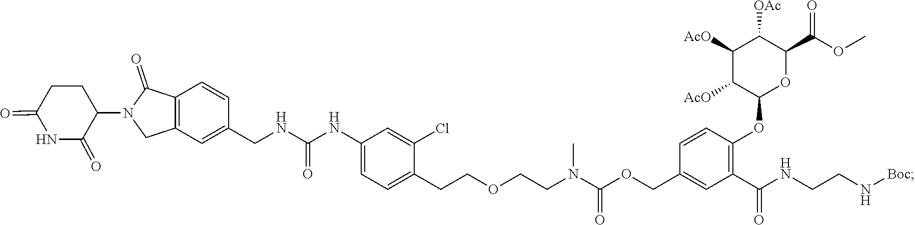 [SMILES: COC(=O)[C@H]1O[C@@H](Oc2ccc(COC(=O)N(C)CCOCCc3ccc(NC(=O)NCc4ccc5c(c4)CN(C4CCC(=O)NC4=O)C5=O)cc3Cl)cc2C(=O)NCCNC(=O)OC(C)(C)C)[C@H](OC(C)=O)[C@@H](OC(C)=O)[C@@H]1OC(C)=O]
(e) a process for preparing compound 18 comprising converting compound 17 to compound 18; wherein
compound 17 is: 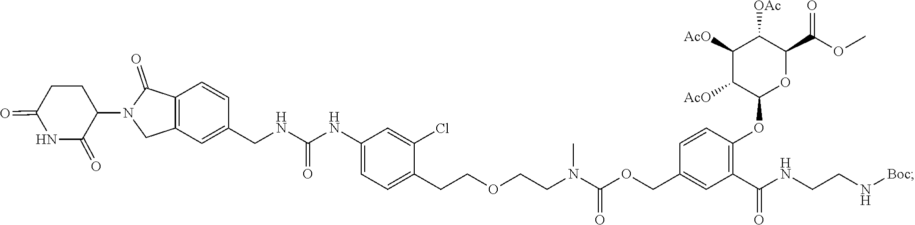 [SMILES: COC(=O)[C@H]1O[C@@H](Oc2ccc(COC(=O)N(C)CCOCCc3ccc(NC(=O)NCc4ccc5c(c4)CN(C4CCC(=O)NC4=O)C5=O)cc3Cl)cc2C(=O)NCCNC(=O)OC(C)(C)C)[C@H](OC(C)=O)[C@@H](OC(C)=O)[C@@H]1OC(C)=O]
and
compound 18 is: 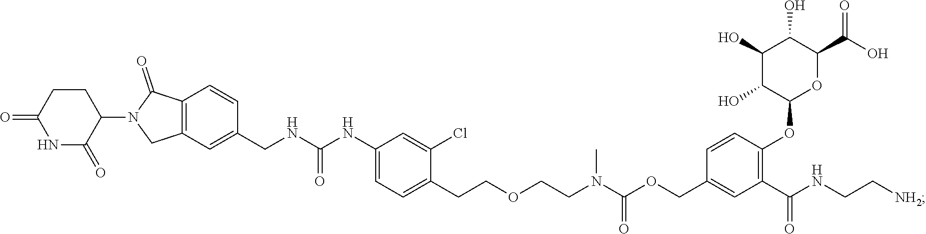 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCN)c1]
and
(f) a process for preparing a compound of formula (II) by reacting compound 18 with compound 19, thereby producing the compound of formula (I-1); where
Formula (II) is: 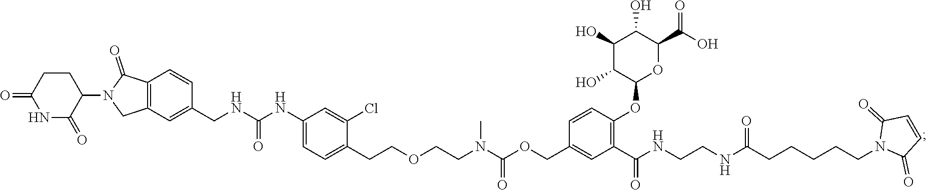 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCNC(=O)CCCCCN2C(=O)C=CC2=O)c1]
Compound 18 is: 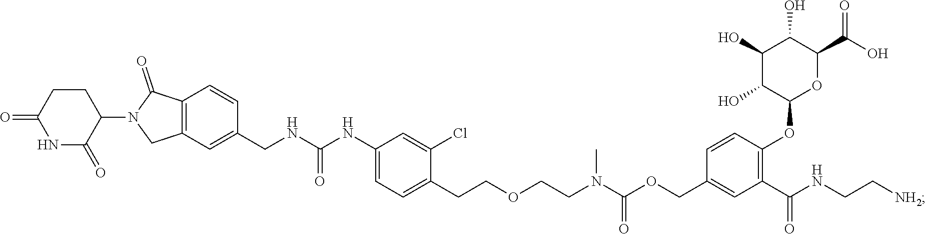 [SMILES: CN(CCOCCc1ccc(NC(=O)NCc2ccc3c(c2)CN(C2CCC(=O)NC2=O)C3=O)cc1Cl)C(=O)OCc1ccc(O[C@@H]2O[C@H](C(=O)O)[C@@H](O)[C@H](O)[C@H]2O)c(C(=O)NCCN)c1]
and
Compound 19 is: 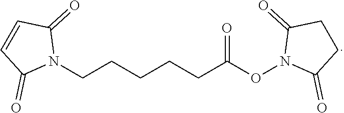 [SMILES: O=C(CCCCCN1C(=O)C=CC1=O)ON1C(=O)CCC1=O]
